(19) United States
(12) Patent Application Publication
(10) Pub. No.: US 2025/0320186 A1
(43) Pub. Date: Oct. 16, 2025

(54) HSD17B13 INHIBITORS AND USES THEREOF

(71) Applicant: Inipharm, Inc., Bellevue, WA (US)

(72) Inventors: Joshua ODINGO, Bothell, WA (US); Sampath Kumar ANANDAN, Fremont, CA (US); Heather Kay Webb HSU, Seattle, WA (US)

(21) Appl. No.: 18/865,617

(22) PCT Filed: May 16, 2023
(86) PCT No.: PCT/US2023/022366

§ 371 Date: Nov. 13, 2024

Related U.S. Application Data
(60) Provisional application No. 63/342,782, filed on May 17, 2022.
(60) Provisional application No. 63/342,792, filed on May 17, 2022.
(60) Provisional application No. 63/342,797, filed on May 17, 2022.
(60) Provisional application No. 63/342,800, filed on May 17, 2022.

(51) Int. Cl. C07D 249/18; C07D 209/12; C07D 209/34; C07D 215/48; C07D 217/26; C07D 231/56; C07D 235/06; C07D 235/26; C07D 263/57; C07D 263/58; C07D 277/34; C07D 277/68; C07D 333/22; C07D 401/04; C07D 401/06; C07D 401/12; C07D 403/04; C07D 409/04; C07D 413/04; C07D 413/06; C07D 413/14; C07D 417/06; C07D 417/12; C07D 417/14; C07D 471/04; C07D 487/04; C07D 498/04; C07D 513/04

(52) U.S. Cl. CPC C07D 249/18; C07D 209/12; C07D 209/34; C07D 215/48; C07D 217/26; C07D 231/56; C07D 235/06; C07D 235/26; C07D 263/57; C07D 263/58; C07D 277/34; C07D 277/68; C07D 333/22; C07D 401/04; C07D 401/06; C07D 401/12; C07D 403/04; C07D 409/04; C07D 413/04; C07D 413/06; C07D 413/14; C07D 417/06; C07D 417/12; C07D 417/14; C07D 471/04; C07D 487/04; C07D 498/04; C07D 513/04

(57) ABSTRACT

Described herein are HSD17B13 inhibitors and pharmaceutical compositions comprising said inhibitors. The subject compounds and compositions are useful for the treatment of liver disease, metabolic disease, or cardiovascular disease, such as NAFLD or NASH, or drug induced liver injury (DILI).

### CROSS-REFERENCE

This application claims the benefit of U.S. Provisional Application Ser. No. 63/342,782 filed May 17, 2022; U.S. Provisional Application Ser. No. 63/342,792 filed May 17, 2022; U.S. Provisional Application Ser. No. 63/342,797 filed May 17, 2022; and U.S. Provisional Application Ser. No. 63/342,800 filed May 17, 2022; which are hereby incorporated by reference in their entirety.

### BACKGROUND OF THE INVENTION

Nonalcoholic fatty liver diseases (NAFLDs) including NASH (nonalcoholic steatohepatitis) are considered to be hepatic manifestations of the metabolic syndrome and are characterized by the accumulation of triglycerides in the liver of patients without a history of excessive alcohol consumption. The majority of patients with NAFLD are obese or morbidly obese and have accompanying insulin resistance. The incidence of NAFLD/NASH has been rapidly increasing worldwide consistent with the increased prevalence of obesity, and it is currently the most common chronic liver disease.

NAFLD is classified into simple steatosis, in which only hepatic steatosis is observed, and NASH, in which intralobular inflammation and ballooning degeneration of hepatocytes is observed along with hepatic steatosis. The proportion of patients with NAFLD who have NASH is still not clear but might range from 20-40%. NASH is a progressive disease and may lead to liver cirrhosis and hepatocellular carcinoma. Twenty percent of NASH patients are reported to develop cirrhosis, and 30-40% of patients with NASH cirrhosis experience liver-related death. Recently, NASH has become the third most common indication for liver transplantation in the United States. Currently, the principal treatment for NAFLD/NASH is lifestyle modification by diet and exercise. However, pharmacological therapy is indispensable because obese patients with NAFLD often have difficulty maintaining improved lifestyles.

17β-Hydroxysteroid dehydrogenases (HSD17Bs) comprise a large family of 15 members some of which involved in sex hormone metabolism. Some HSD17Bs enzymes also play key roles in cholesterol and fatty acid metabolism. A recent study showed that hydroxysteroid 17β-dehydrogenase 13 (HSD17B13), an enzyme with unknown biological function, is a novel liver-specific lipid droplet (LD)-associated protein in mouse and humans. HSD17B13 expression is markedly upregulated in patients and mice with non-alcoholic fatty liver disease (NAFLD). Hepatic overexpression of HSD17B13 promotes lipid accumulation in the liver. HSD17B13 could also have potential as a biomarker of chronic liver disease, such as alcoholic liver disease (ALD), non-alcoholic fatty liver disease (NAFLD) (for example: steatosis, nonalcoholic steatohepatitis (NASH), NASH-fibrosis, or cirrhosis), steatohepatitis, and liver cancer.

### SUMMARY OF THE INVENTION

Provided herein are methods, compounds, and compositions useful for reducing expression or activity of HSD17B13 in a subject in need thereof. Also, provided herein are methods, compounds, and compositions comprising HSD17B13 specific inhibitors, which can be useful in reducing the morbidity of HSD17B13-related diseases or conditions in a subject in need thereof. Such methods, compounds, and compositions can be useful, for example, to treat, prevent, delay, or ameliorate liver disease, metabolic disease, or cardiovascular disease.

Disclosed herein is a compound of Formula (I), or a pharmaceutically acceptable salt, solvate, or stereoisomer thereof:

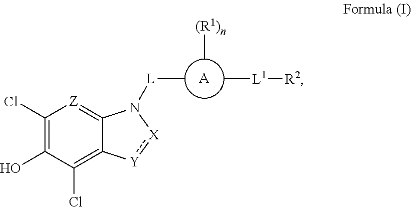

wherein:
Z is N or CR^{Z};
R^{Z} is hydrogen, deuterium, halogen, —CN, —OH, —OR^{a}, —NR^{c}R^{d}, or C₁-C₆alkyl optionally substituted with one or more R;
[symbol] is a single bond;
X is —C(R⁴)₂— or —C(═NR³)—;
Y is —O—, —NR³—, or —C(R⁴)₂—;
or
[symbol] is a double bond;
X is —N— or —CR⁵—;
Y is —N— or —CR⁵—;
R³ is hydrogen or C₁-C₆alkyl optionally substituted with one or more R;
each R⁴ is independently hydrogen, deuterium, halogen, —CN, —OH, —OR^{a}, —NR^{c}R^{d}, or C₁-C₆alkyl optionally substituted with one or more R;
or two R⁴ are taken together to form an oxo;
each R⁵ is independently hydrogen, deuterium, halogen, —CN, —OH, —OR^{a}, —NR^{c}R^{d}, or C₁-C₆alkyl optionally substituted with one or more R;
L is absent, —C(R⁷)₂—, —C(═O)—, —S(═O)—, —S(═O)₂—, —C(R)₂—NR⁶—, or —C(═O)—NR⁶—;
R⁶ is hydrogen or C₁-C₆alkyl optionally substituted with one or more R;
each R⁷ is independently hydrogen, deuterium, halogen, —CN, —OH, —OR^{a}, —NR^{c}R^{d}, or C₁-C₆alkyl optionally substituted with one or more R;
or two R⁷ are taken together to form an oxo;
or two R⁷ are taken together to form a cycloalkyl or heterocycloalkyl; each optionally substituted with one or more R;
Ring A is a cycloalkyl, heterocycloalkyl, aryl, or heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R¹ on the same atom are taken together to form an oxo;
n is 0-4;
L¹ is absent, —O—, —S—, —NR⁶—, —C(═O)—, —C(═O)O—, —S(═O)—, —S(═O)₂—, —NR⁶—C(R⁷)₂—, —C(R⁷)₂—NR⁶—, —NR⁶—C(═O)—, or —C(═O)—NR⁶—;
R² is C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{2a};
each R^{2a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{2a} on the same atom are taken together to form an oxo;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

Also disclosed herein is a compound of Formula (II), or a pharmaceutically acceptable salt, solvate, or stereoisomer thereof:

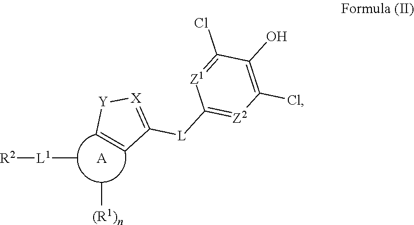

wherein:
Ring A is phenyl or 6-membered heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
n is 0-3;
L¹ is absent, —O—, —S—, —NR⁶—, —C(═O)—, —C(═O)O—, —S(═O)—, —S(═O)₂—, —NR⁶—C(R⁷)₂—, —C(R⁷)₂—NR⁶—, —NR⁶—C(═O)—, or —C(═O)—NR⁶—;
R² is cycloalkyl, heterocycloalkyl, aryl, or heteroaryl; wherein the cycloalkyl, heterocycloalkyl, aryl, and heteroaryl is optionally substituted with one or more R^{2a};
each R^{2a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{2a} on the same atom are taken together to form an oxo;
X is —N— or —CR³—;
Y is —O—, —NR⁴—, or —C(R⁵)₂—;
R³ is hydrogen, deuterium, halogen, —CN, —OH, —OR^{a}, —NR^{c}R^{d}, or C₁-C₆alkyl optionally substituted with one or more R;
R⁴ is hydrogen or C₁-C₆alkyl optionally substituted with one or more R;
each R⁵ is independently hydrogen, deuterium, halogen, —CN, —OH, —OR^{a}, —NR^{c}R^{d}, or C₁-C₆alkyl optionally substituted with one or more R;
or two R⁵ are taken together to form an oxo;
L is absent, —O—, —S—, —NR⁶—, —C(R⁷)₂—, —C(═O)—, —S(═O)—, —S(═O)₂—, —NR⁶—C(R⁷)₂—, —C(R⁷)₂—NR⁶—, —NR⁶—C(═O)—, —C(═O)—NR⁶—, or —NR⁶—C(═O)—NR⁶—;
R⁶ is hydrogen or C₁-C₆alkyl optionally substituted with one or more R;
each R⁷ is independently hydrogen, deuterium, halogen, —CN, —OH, —OR^{a}, —NR^{c}R^{d}, or C₁-C₆alkyl optionally substituted with one or more R;
or two R⁷ are taken together to form an oxo;
or two R⁷ are taken together to form a cycloalkyl or heterocycloalkyl; each optionally substituted with one or more R;
Z¹ is N or CR^{Z1};
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z2};
R^{Z2} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo;
provided that the compound is not:

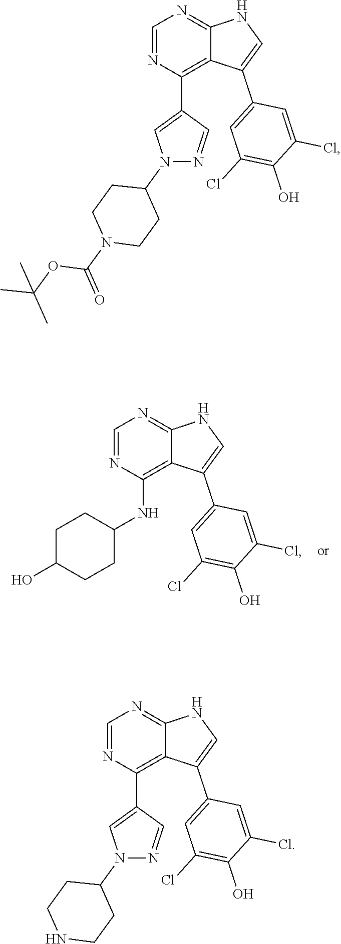 [SMILES: CC(C)(C)OC(=O)N1CCC(n2cc(-c3ncnc4[nH]cc(-c5cc(Cl)c(O)c(Cl)c5)c34)cn2)CC1 ; Oc1c(Cl)cc(-c2c[nH]c3ncnc(NC4CCC(O)CC4)c23)cc1Cl ; Oc1c(Cl)cc(-c2c[nH]c3ncnc(-c4cnn(C5CCNCC5)c4)c23)cc1Cl]

Also disclosed herein is a compound of Formula (III), or a pharmaceutically acceptable salt, solvate, or stereoisomer thereof:

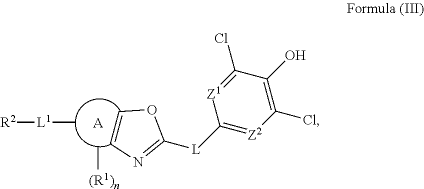

wherein:
Ring A is aryl or 6-membered heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
n is 0-3;
L¹ is absent, —O—, —S—, —NR⁶—, —C(═O)—, —C(═O)O—, —S(═O)—, —S(═O)₂—, —NR⁶—C(R⁷)₂—, —C(R⁷)₂—NR⁶—, —NR⁶—C(═O)—, or —C(═O)—NR⁶—;
R² is cycloalkyl, heterocycloalkyl, aryl, or heteroaryl; wherein the cycloalkyl, heterocycloalkyl, aryl, and heteroaryl is optionally substituted with one or more R^{2a};
each R^{2a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{2a} on the same atom are taken together to form an oxo;
L is absent, —O—, —S—, —NR⁶—, —C(R⁷)₂—, —C(═O)—, —S(═O)—, —S(═O)₂—, —NR⁶—C(R⁷)₂—, —C(R⁷)₂—NR⁶—, —NR⁶—C(═O)—, —C(═O)—NR⁶—, or —NR⁶—C(═O)—NR⁶—;
R⁶ is hydrogen or C₁-C₆alkyl optionally substituted with one or more R;
each R⁷ is independently hydrogen, deuterium, halogen, —CN, —OH, —OR^{a}, —NR^{c}R^{d}, or C₁-C₆alkyl optionally substituted with one or more R;
or two R⁷ are taken together to form an oxo;
Z¹ is N or CR^{Z1};
R^{Z1} is hydrogen, deuterium, halogen, —CN, —OH, —OR^{a}, —NR^{c}R^{d}, or C₁-C₆alkyl optionally substituted with one or more R; Z² is N or CR^{Z2};
R^{Z2} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

Also disclosed herein is a compound of Formula (IV), or a pharmaceutically acceptable salt, solvate, or stereoisomer thereof:

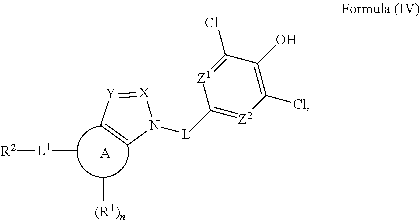

wherein:
Ring A is phenyl or 6-membered heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
n is 0-3;
L¹ is absent, —O—, —S—, —NR⁶—, —C(═O)—, —C(═O)O—, —S(═O)—, —S(═O)₂—, —NR⁶—C(R⁷)₂—, —C(R⁷)₂—NR⁶—, —NR⁶—C(═O)—, or —C(═O)—NR⁶—;
R² is cycloalkyl, heterocycloalkyl, aryl, or heteroaryl; wherein the cycloalkyl, heterocycloalkyl, aryl, and heteroaryl is optionally substituted with one or more R^{2a};
each R^{2a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{2a} on the same atom are taken together to form an oxo;
X is —N— or —CR^{X}—;
R^{X} is hydrogen, deuterium, halogen, —CN, —OH, —OR^{a}, —NR^{c}R^{d}, or C₁-C₆alkyl optionally substituted with one or more R;
Y is —N— or —CR^{Y}—;
R^{Y} is hydrogen, deuterium, halogen, —CN, —OH, —OR^{a}, —NR^{c}R^{d}, or C₁-C₆alkyl optionally substituted with one or more R;
L is absent, —C(R⁷)₂—, —C(═O)—, —S(═O)—, —S(═O)₂—, —C(R⁷)₂—NR⁶—, or —C(═O)—NR⁶—;
R⁶ is hydrogen or C₁-C₆alkyl optionally substituted with one or more R;
each R⁷ is independently hydrogen, deuterium, halogen, —CN, —OH, —OR^{a}, —NR^{c}R^{d}, or C₁-C₆alkyl optionally substituted with one or more R;
or two R⁷ are taken together to form an oxo;
or two R⁷ are taken together to form a cycloalkyl or heterocycloalkyl; each optionally substituted with one or more R;
Z¹ is N or CR^{Z1};
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z2};
R^{Z2} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

Also disclosed herein is a compound of Formula (V), or a pharmaceutically acceptable salt, solvate, or stereoisomer thereof:

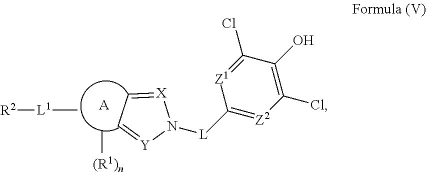

wherein:
Ring A is phenyl or 6-membered heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
n is 0-3;
L¹ is absent, —O—, —S—, —NR⁶—, —C(═O)—, —C(═O)O—, —S(═O)—, —S(═O)₂—, —NR⁶—C(R⁷)₂—, —C(R⁷)₂—NR⁶—, —NR⁶—C(═O)—, or —C(═O)—NR⁶—; R² is cycloalkyl, heterocycloalkyl, aryl, or heteroaryl; wherein the cycloalkyl, heterocycloalkyl, aryl, and heteroaryl is optionally substituted with one or more R^{2a};
each R^{2a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{2a} on the same atom are taken together to form an oxo;
X is —N— or —CR^{X}—;
R^{X} is hydrogen, deuterium, halogen, —CN, —OH, —OR^{a}, —NR^{c}R^{d}, or C₁-C₆alkyl optionally substituted with one or more R;
Y is —N— or —CR^{Y}—;
R^{Y} is hydrogen, deuterium, halogen, —CN, —OH, —OR^{a}, —NR^{c}R^{d}, or C₁-C₆alkyl optionally substituted with one or more R;
L is absent, —C(R⁷)₂—, —C(═O)—, —S(═O)—, —S(═O)₂—, —C(R)₂—NR⁶—, or —C(═O)—NR⁶—;
R⁶ is hydrogen or C₁-C₆alkyl optionally substituted with one or more R;
each R⁷ is independently hydrogen, deuterium, halogen, —CN, —OH, —OR^{a}, —NR^{c}R^{d}, or C₁-C₆alkyl optionally substituted with one or more R;
or two R⁷ are taken together to form an oxo;
or two R⁷ are taken together to form a cycloalkyl or heterocycloalkyl; each optionally substituted with one or more R;
Z¹ is N or CR^{Z1};
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z2};
R^{Z2} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

Also disclosed herein is a compound of Formula (VI), or a pharmaceutically acceptable salt, solvate, or stereoisomer thereof:

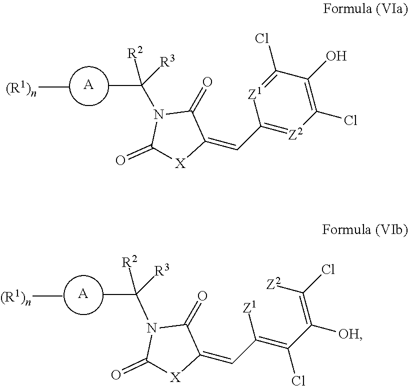

wherein:
Ring A is cycloalkyl, heterocycloalkyl, aryl, or heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or 2 R¹ on the same atoms are taken together to form an oxo;
n is 0-4;
R² is hydrogen, deuterium, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
R³ is hydrogen, deuterium, or C₁-C₆alkyl;
X is —O— or —S—;
Z¹ is N or CR^{Z1};
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z2};
R^{Z2} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

Also disclosed herein is a compound of Formula (VII), or a pharmaceutically acceptable salt, solvate, or stereoisomer thereof:

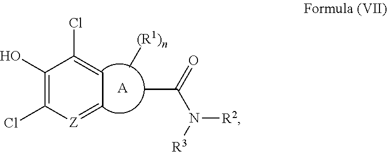

wherein:
Ring A is cycloalkyl, heterocycloalkyl, aryl, or heteroaryl;
each R¹ is independently deuterium, halogen, —CN, —NO₂, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R¹ on the same atom are taken together to form an oxo;
n is 0-4;
R² is hydrogen or C₁-C₆alkyl;
R³ is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, alkenyl, alkynyl, cycloalkyl, heterocycloalkyl, aryl, and heteroaryl is optionally substituted with one or more R^{3a};
each R^{3a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{3a} on the same atom are taken together to form an oxo;
Z is N or CR^{Z};
R^{Z} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

Also disclosed herein is a compound of Formula (VIIIa) or (VIIIb), or a pharmaceutically acceptable salt, solvate, or stereoisomer thereof:

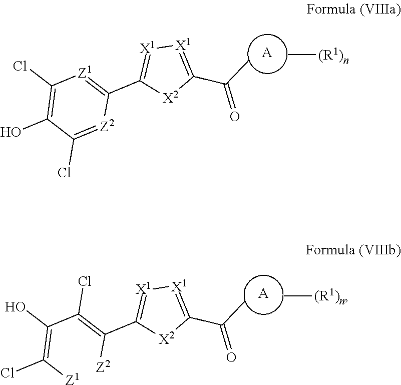

wherein:
Ring A is cycloalkyl, heterocycloalkyl, aryl, or heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R¹ on the same atom are taken together to form an oxo;
n is 0-4;
Z¹ is N or CR^{Z1};
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z1};
R^{Z2} is hydrogen, deuterium, halogen, —CN, —OH, —OR^{a}, —NR^{c}R^{d}, or C₁-C₆alkyl optionally substituted with one or more R;
each X¹ is independently —N— or —CR^{X1}—;
each R^{X1} is hydrogen, deuterium, halogen, —CN, —OH, —OR^{a}, —NR^{c}R^{d}, or C₁-C₆alkyl optionally substituted with one or more R;
or two R^{X1} are taken together to form a cycloalkyl, heterocycloalkyl, aryl, or heteroaryl; each optionally substituted with one or more R;
X² is —O—, —S—, or —NR^{X2}—;
R^{X2} is hydrogen or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo;
provided that the compound is not:

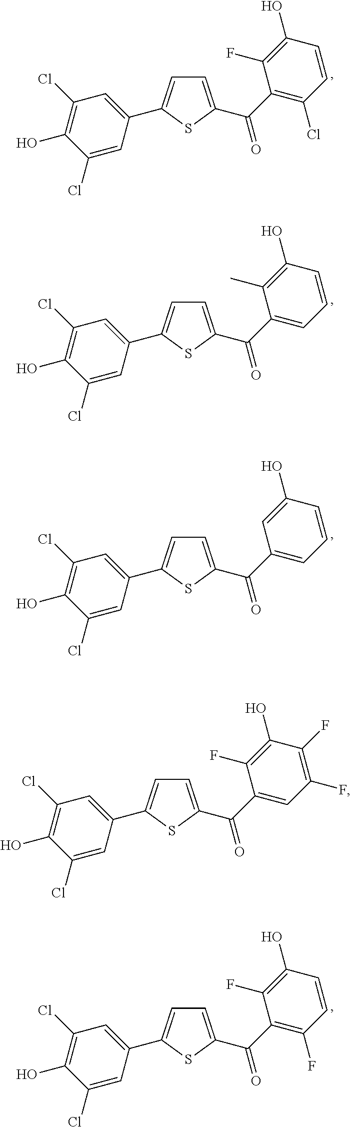 [SMILES: O=C(c1ccc(-c2cc(Cl)c(O)c(Cl)c2)s1)c1c(Cl)ccc(O)c1F ; Cc1c(O)cccc1C(=O)c1ccc(-c2cc(Cl)c(O)c(Cl)c2)s1 ; O=C(c1cccc(O)c1)c1ccc(-c2cc(Cl)c(O)c(Cl)c2)s1 ; O=C(c1ccc(-c2cc(Cl)c(O)c(Cl)c2)s1)c1cc(F)c(F)c(O)c1F ; O=C(c1ccc(-c2cc(Cl)c(O)c(Cl)c2)s1)c1c(F)ccc(O)c1F]

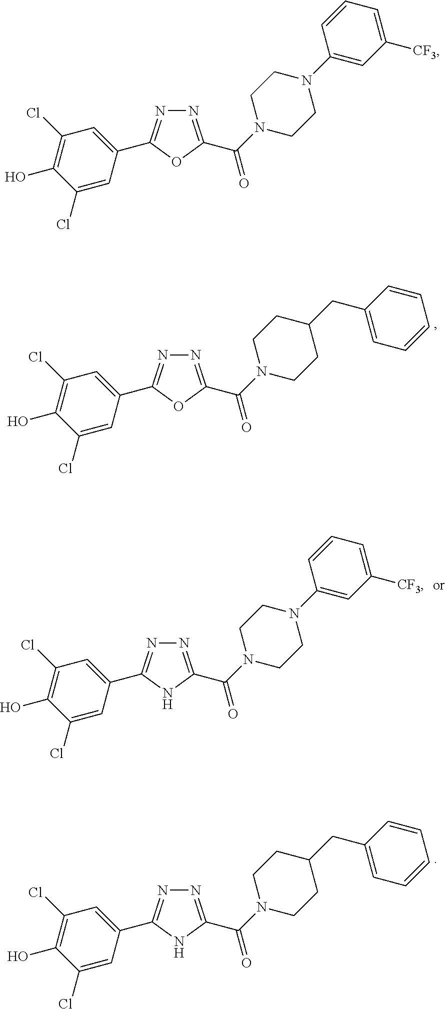 [SMILES: O=C(c1nnc(-c2cc(Cl)c(O)c(Cl)c2)o1)N1CCN(c2cccc(C(F)(F)F)c2)CC1 ; O=C(c1nnc(-c2cc(Cl)c(O)c(Cl)c2)o1)N1CCC(Cc2ccccc2)CC1 ; O=C(c1nnc(-c2cc(Cl)c(O)c(Cl)c2)[nH]1)N1CCN(c2cccc(C(F)(F)F)c2)CC1 ; O=C(c1nnc(-c2cc(Cl)c(O)c(Cl)c2)[nH]1)N1CCC(Cc2ccccc2)CC1]

Also disclosed herein is a pharmaceutical composition comprising a compound disclosed herein, or a pharmaceutically acceptable salt, solvate, or stereoisomer thereof, and a pharmaceutically acceptable carrier.

Also disclosed herein is a method of treating a disease in a subject in need thereof, the method comprising administering a pharmaceutically effective amount of a compound disclosed herein, or a pharmaceutically acceptable salt, solvate, or stereoisomer thereof, or a pharmaceutical composition disclosed herein. In some embodiments of a method of treating a disease, the disease is a liver disease, a metabolic disease, or a cardiovascular disease. In some embodiments of a method of treating a disease, the disease is NAFLD. In some embodiments of a method of treating a disease, the disease is NASH. In some embodiments of a method of treating a disease, the disease is drug induced liver injury (DILI). In some embodiments of a method of treating a disease, the disease is associated with HSD17B13. In some embodiments of a method of treating a disease, the diseases is alcoholic liver disease. In some embodiments of a method of treating a disease, the disease is cirrhosis. In some embodiments of a method of treating a disease, the disease is decompensated portal hypertension. In some embodiments of a method of treating a disease, the disease is cholestatic liver disease.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl. In some embodiments, the alkyl is a C₁-C₆ alkyl. In some embodiments, the alkyl is a C₁-C₅ alkyl. In some embodiments, the alkyl is a C₁-C₄ alkyl. In some embodiments, the alkyl is a C₁-C₃ alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)—CH₂], butenyl, 1,3-butadienyl and the like. Whenever it appears herein, a numerical range such as “C₂-C₆ alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —Oalkyl where alkyl is defined as above. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom) or bridged ring systems. In some embodiments, the cycloalkyl is fully saturated. Representative cycloalkyls include, but are not limited to, cycloalkyls having from three to fifteen carbon atoms (C₃-C₁₅ cycloalkyl or C₃-C₁₅ cycloalkenyl), from three to ten carbon atoms (C₃-C₁₀ cycloalkyl or C₃-C₁₀ cycloalkenyl), from three to eight carbon atoms (C₃-C₈ cycloalkyl or C₃-C₈ cycloalkenyl), from three to six carbon atoms (C₃-C₆ cycloalkyl or C₃-C₆ cycloalkenyl), from three to five carbon atoms (C₃-C₅ cycloalkyl or C₃-C₅ cycloalkenyl), or three to four carbon atoms (C₃-C₄ cycloalkyl or C₃-C₄ cycloalkenyl). In some embodiments, the cycloalkyl is a 3- to 10-membered cycloalkyl or a 3- to 10-membered cycloalkenyl. In some embodiments, the cycloalkyl is a 3- to 6-membered cycloalkyl or a 3- to 6-membered cycloalkenyl. In some embodiments, the cycloalkyl is a 5- to 6-membered cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Heteroalkyl” refers to an alkyl group in which one or more skeletal atoms of the alkyl are selected from an atom other than carbon, e.g., oxygen, nitrogen, sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen, sulfur, phosphorus, or combinations thereof wherein the heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or two atoms selected from the group consisting of oxygen, nitrogen, and sulfur wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl or C₂-C₁₅ heterocycloalkenyl), from two to ten carbon atoms (C₂-C₁₀ heterocycloalkyl or C₂-C₁₀ heterocycloalkenyl), from two to eight carbon atoms (C₂-C₈ heterocycloalkyl or C₂-C₈ heterocycloalkenyl), from two to seven carbon atoms (C₂-C₇ heterocycloalkyl or C₂-C₇ heterocycloalkenyl), from two to six carbon atoms (C₂-C₆ heterocycloalkyl or C₂-C₆ heterocycloalkenyl), from two to five carbon atoms (C₂-C₅ heterocycloalkyl or C₂-C₅ heterocycloalkenyl), or two to four carbon atoms (C₂-C₄ heterocycloalkyl or C₂-C₄ heterocycloalkenyl). In some embodiments, the heterocycloalkyl is a 3- to 8-membered ring comprising 1, 2, or 3 heteroatoms selected from the group consisting of oxygen, nitrogen, or sulfur. In some embodiments, the heterocycloalkyl is a 3- to 6-membered ring comprising 1, 2, or 3 heteroatoms selected from the group consisting of oxygen, nitrogen, or sulfur. In some embodiments, the heterocycloalkyl is a 5- to 6-membered ring comprising 1, 2, or 3 heteroatoms selected from the group consisting of oxygen, nitrogen, or sulfur. Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. In some embodiments, the heteroaryl is a 5- to 6-membered ring comprising 1, 2, or 3 heteroatoms selected from the group consisting of oxygen, nitrogen, or sulfur. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition. In some embodiments, treatment also includes prophylactic treatment (e.g., administration of a composition described herein when an individual is suspected to be suffering from a liver disease, e.g., NAFLD).

“Synergy” or “synergize” refers to an effect of a combination that is greater than additive of the effects of each component alone at the same doses.

“HSD17B13” means hydroxysteroid 17-beta dehydrogenase 13 and refers to any nucleic acid of HSD17B13. For example, in some embodiments, HSD17B13 includes a DNA sequence encoding HSD17B13, an RNA sequence transcribed from DNA encoding HSD17B13 (including genomic DNA comprising introns and exons). HSD17B13 can also refer to any amino acid sequence of HSD17B13 (may include secondary or tertiary structures of the protein molecule), encoded by a DNA sequence and/or RNA sequence. The target may be referred to in either upper or lower case.

### Compounds

Described herein are compounds, or a pharmaceutically acceptable salt, solvate, or stereoisomer thereof useful in the treatment of liver diseases. In some embodiments, the liver disease is NAFLD.

Disclosed herein is a compound of Formula (I), or a pharmaceutically acceptable salt, solvate, or stereoisomer thereof:

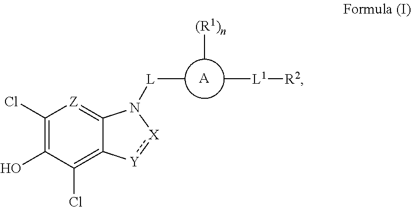

wherein:
Z is N or CR^{Z};
R^{Z} is hydrogen, deuterium, halogen, —CN, —OH, —OR^{a}, —NR^{c}R^{d}, or C₁-C₆alkyl optionally substituted with one or more R;
[symbol] is a single bond;
X is —C(R⁴)₂— or —C(═NR³)—;
Y is —O—, —NR³—, or —C(R⁴)₂—;
or
[symbol] is a double bond;
X is —N— or —CR⁵—;
Y is —N— or —CR⁵—;
R³ is hydrogen or C₁-C₆alkyl optionally substituted with one or more R;
each R⁴ is independently hydrogen, deuterium, halogen, —CN, —OH, —OR^{a}, —NR^{c}R^{d}, or C₁-C₆alkyl optionally substituted with one or more R;
or two R⁴ are taken together to form an oxo;
each R⁵ is independently hydrogen, deuterium, halogen, —CN, —OH, —OR^{a}, —NR^{c}R^{d}, or C₁-C₆alkyl optionally substituted with one or more R;
L is absent, —C(R⁷)₂—, —C(═O)—, —S(═O)—, —S(═O)₂—, —C(R⁷)₂—NR⁶—, or —C(═O)—NR⁶—;
R⁶ is hydrogen or C₁-C₆alkyl optionally substituted with one or more R;
each R⁷ is independently hydrogen, deuterium, halogen, —CN, —OH, —OR^{a}, —NR^{c}R^{d}, or C₁-C₆alkyl optionally substituted with one or more R;
or two R⁷ are taken together to form an oxo;
or two R⁷ are taken together to form a cycloalkyl or heterocycloalkyl; each optionally substituted with one or more R;
Ring A is a cycloalkyl, heterocycloalkyl, aryl, or heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R¹ on the same atom are taken together to form an oxo;
n is 0-4;
L¹ is absent, —O—, —S—, —NR⁶—, —C(═O)—, —C(═O)O—, —S(═O)—, —S(═O)₂—, —NR⁶—C(R⁷)₂—, —C(R⁷)₂—NR⁶—, —NR⁶—C(═O)—, or —C(═O)—NR⁶—;
R² is C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{2a};
each R^{2a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{2a} on the same atom are taken together to form an oxo;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

In some embodiments of a compound of Formula (I), [symbol] is a single bond and X is —C(R⁴)₂—. In some embodiments of a compound of Formula (I), [symbol] is a single bond and X is —C(=NR³)—. In some embodiments of a compound of Formula (I), [symbol] is a single bond and Y is —O—. In some embodiments of a compound of Formula (I), [symbol] is a single bond and Y is —NR³—. In some embodiments of a compound of Formula (I), [symbol] is a single bond and Y is —C(R⁴)₂—.

In some embodiments of a compound of Formula (I), [symbol] is a double bond and X is —N—. In some embodiments of a compound of Formula (I), [symbol] is a double bond and X is —CR⁵—. In some embodiments of a compound of Formula (I), [symbol] is a double bond and Y is —N—. In some embodiments of a compound of Formula (I), [symbol] is a double bond and Y is —CR⁵—.

In some embodiments of a compound of Formula (I), the compound is of Formula (Ia):

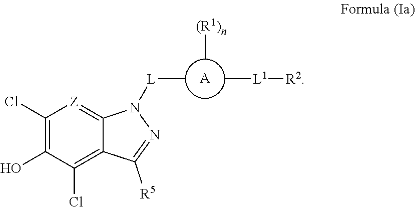

In some embodiments of a compound of Formula (I), the compound is of Formula (Ib):

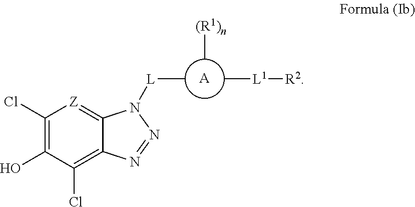

In some embodiments of a compound of Formula (I), the compound is of Formula (Ic):

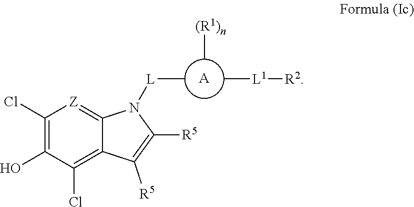

In some embodiments of a compound of Formula (I), the compound is of Formula (Id):

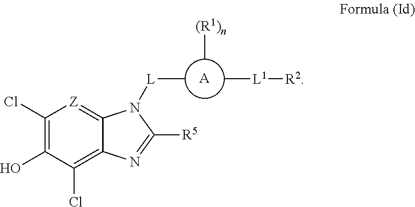

In some embodiments of a compound of Formula (I), the compound is of Formula (Ie):

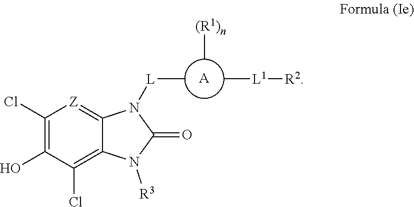

In some embodiments of a compound of Formula (I), the compound is of Formula (If):

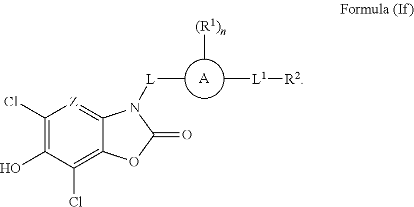

In some embodiments of a compound of Formula (I), the compound is of Formula (Ig):

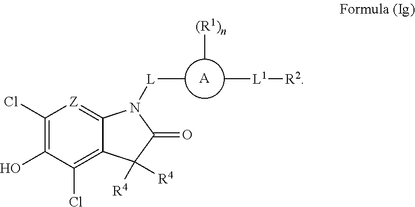

In some embodiments of a compound of Formula (I) or (Ie), R³ is hydrogen or C₁-C₆alkyl. In some embodiments of a compound of Formula (I) or (Ie), R³ is hydrogen.

In some embodiments of a compound of Formula (I) or (Ig), each R⁴ is independently hydrogen, deuterium, halogen, C₁-C₆alkyl, C₁-C₆haloalkyl, or C₁-C₆deuteroalkyl. In some embodiments of a compound of Formula (I) or (Ig), each R⁴ is hydrogen. In some embodiments of a compound of Formula (I) or (Ig), two R⁴ are taken together to form an oxo.

In some embodiments of a compound of Formula (I), (Ia), (Ic), or (Id), each R⁵ is independently hydrogen, deuterium, halogen, C₁-C₆alkyl, C₁-C₆haloalkyl, or C₁-C₆deuteroalkyl. In some embodiments of a compound of Formula (I), (Ia), (Ic), or (Id), each R⁵ is independently hydrogen, deuterium, halogen, or C₁-C₆alkyl. In some embodiments of a compound of Formula (I), (Ia), (Ic), or (Id), each R⁵ is independently hydrogen, halogen, or C₁-C₆alkyl. In some embodiments of a compound of Formula (I), (Ia), (Ic), or (Id), each R⁵ is hydrogen.

In some embodiments of a compound of Formula (I) or (Ia)-(Ig), Z is N. In some embodiments of a compound of Formula (I) or (Ia)-(Ig), Z is CR^{Z}.

In some embodiments of a compound of Formula (I) or (Ia)-(Ig), R^{Z} is hydrogen, deuterium, halogen, C₁-C₆alkyl, C₁-C₆haloalkyl, or C₁-C₆deuteroalkyl. In some embodiments of a compound of Formula (I) or (Ia)-(Ig), R^{Z} is hydrogen, halogen, or C₁-C₆alkyl. In some embodiments of a compound of Formula (I) or (Ia)-(Ig), R^{Z} is hydrogen.

In some embodiments of a compound of Formula (I) or (Ia)-(Ig), L is absent. In some embodiments of a compound of Formula (I) or (Ia)-(Ig), L is —C(R⁷)₂—. In some embodiments of a compound of Formula (I) or (Ia)-(Ig), L is —C(═O)—. In some embodiments of a compound of Formula (I) or (Ia)-(Ig), L is —S(═O)— or —S(═O)₂—. In some embodiments of a compound of Formula (I) or (Ia)-(Ig), L is —C(R⁷)₂—NR⁶—. In some embodiments of a compound of Formula (I) or (Ia)-(Ig), L is —C(═O)—NR⁶—.

In some embodiments of a compound of Formula (I) or (Ia)-(Ig), R⁶ is hydrogen or C₁-C₆alkyl. In some embodiments of a compound of Formula (I) or (Ia)-(Ig), R⁶ is hydrogen.

In some embodiments of a compound of Formula (I) or (Ia)-(Ig), each R⁷ is independently hydrogen, deuterium, halogen, C₁-C₆alkyl, C₁-C₆haloalkyl, or C₁-C₆deuteroalkyl. In some embodiments of a compound of Formula (I) or (Ia)-(Ig), each R⁷ is hydrogen. In some embodiments of a compound of Formula (I) or (Ia)-(Ig), two R⁷ are taken together to form an oxo.

In some embodiments of a compound of Formula (I) or (Ia)-(Ig), Ring A is a aryl or heteroaryl. In some embodiments of a compound of Formula (I) or (Ia)-(Ig), Ring A is phenyl. In some embodiments of a compound of Formula (I) or (Ia)-(Ig), Ring A is a 5- or 6-membered heteroaryl. In some embodiments of a compound of Formula (I) or (Ia)-(Ig), Ring A is a 6-membered heteroaryl. In some embodiments of a compound of Formula (I) or (Ia)-(Ig), Ring A is pyridinyl, pyrimidinyl, or pyrazinyl. In some embodiments of a compound of Formula (I) or (Ia)-(Ig), Ring A is pyridinyl or pyrimidinyl. In some embodiments of a compound of Formula (I) or (Ia)-(Ig), Ring A is pyridinyl. In some embodiments of a compound of Formula (I) or (Ia)-(Ig), Ring A is pyrimidinyl.

In some embodiments of a compound of Formula (I) or (Ia)-(Ig), each R¹ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl. In some embodiments of a compound of Formula (I) or (Ia)-(Ig), each R¹ is independently deuterium, halogen, C₁-C₆alkyl, or C₁-C₆haloalkyl. In some embodiments of a compound of Formula (I) or (Ia)-(Ig), each R¹ is independently halogen or C₁-C₆alkyl.

In some embodiments of a compound of Formula (I) or (Ia)-(Ig), n is 0-2. In some embodiments of a compound of Formula (I) or (Ia)-(Ig), n is 0 or 1. In some embodiments of a compound of Formula (I) or (Ia)-(Ig), n is 0. In some embodiments of a compound of Formula (I) or (Ia)-(Ig), n is 1.

In some embodiments of a compound of Formula (I) or (Ia)-(Ig), L¹ is absent. In some embodiments of a compound of Formula (I) or (Ia)-(Ig), L¹ is —O—, —S—, or —NR⁶—. In some embodiments of a compound of Formula (I) or (Ia)-(Ig), L¹ is —O—. In some embodiments of a compound of Formula (I) or (Ia)-(Ig), L¹ is —S—. In some embodiments of a compound of Formula (I) or (Ia)-(Ig), L¹ is —NR⁶—. In some embodiments of a compound of Formula (I) or (Ia)-(Ig), L¹ is —C(═O)—, —C(═O)O—, —S(═O)—, or —S(═O)₂—. In some embodiments of a compound of Formula (I) or (Ia)-(Ig), L¹ is —C(═O)—. In some embodiments of a compound of Formula (I) or (Ia)-(Ig), L¹ is —C(═O)O—. In some embodiments of a compound of Formula (I) or (Ia)-(Ig), L¹ is —S(═O)—. In some embodiments of a compound of Formula (I) or (Ia)-(Ig), L¹ is —S(═O)₂—. In some embodiments of a compound of Formula (I) or (Ia)-(Ig), L¹ is —NR⁶—C(R⁷)₂— or —C(R⁷)₂—NR⁶—. In some embodiments of a compound of Formula (I) or (Ia)-(Ig), L¹ is —NR⁶—C(R⁷)₂—. In some embodiments of a compound of Formula (I) or (Ia)-(Ig), L¹ is —C(R⁷)₂—NR⁶—. In some embodiments of a compound of Formula (I) or (Ia)-(Ig), L¹ is —NR⁶—C(═O)— or —C(═O)—NR⁶—. In some embodiments of a compound of Formula (I) or (Ia)-(Ig), L¹ is —NR⁶—C(═O)—. In some embodiments of a compound of Formula (I) or (Ia)-(Ig), L¹ is —C(═O)—NR⁶—.

In some embodiments of a compound of Formula (I) or (Ia)-(Ig), R² is cycloalkyl, heterocycloalkyl, aryl, or heteroaryl; wherein the cycloalkyl, heterocycloalkyl, aryl, and heteroaryl is optionally substituted with one or more R^{2a}. In some embodiments of a compound of Formula (I) or (Ia)-(Ig), R² is aryl or heteroaryl; wherein the aryl and heteroaryl is optionally substituted with one or more R^{2a}. In some embodiments of a compound of Formula (I) or (Ia)-(Ig), R² is phenyl optionally substituted with one or more R^{2a}. In some embodiments of a compound of Formula (I) or (Ia)-(Ig), R² is 5- or 6-membered heteroaryl; wherein the heteroaryl is optionally substituted with one or more R^{2a}. In some embodiments of a compound of Formula (I) or (Ia)-(Ig), R² is 6-membered heteroaryl; wherein the heteroaryl is optionally substituted with one or more R^{2a}. In some embodiments of a compound of Formula (I) or (Ia)-(Ig), R² is cycloalkyl or heterocycloalkyl; wherein the cycloalkyl and heterocycloalkyl is optionally substituted with one or more R^{2a}. In some embodiments of a compound of Formula (I) or (Ia)-(Ig), R² is heterocycloalkyl optionally substituted with one or more R^{2a}.

In some embodiments of a compound of Formula (I) or (Ia)-(Ig), each R^{2a} is independently deuterium, halogen, —CN, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R.

In some embodiments of a compound of Formula (I) or (Ia)-(Ig), each R^{2a} is independently deuterium, halogen, —CN, —OH, —OR^{a}, —S(═O)₂R^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl; wherein each alkyl is independently optionally substituted with one or more R.

In some embodiments of a compound of Formula (I) or (Ia)-(Ig), each R^{2a} is independently deuterium, halogen, —CN, —OH, —OR^{a}, —S(═O)₂R^{a}, —NR^{c}R^{d}, C₁-C₆alkyl, or C₁-C₆haloalkyl.

Also disclosed herein is a compound of Formula (II), or a pharmaceutically acceptable salt, solvate, or stereoisomer thereof:

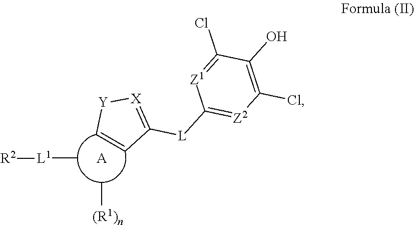

wherein:
Ring A is phenyl or 6-membered heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
n is 0-3;
L¹ is absent, —O—, —S—, —NR⁶—, —C(═O)—, —C(═O)O—, —S(═O)—, —S(═O)₂—, —NR⁶—C(R⁷)₂—, —C(R⁷)₂—NR⁶—, —NR⁶—C(═O)—, or —C(═O)—NR⁶—;
R² is cycloalkyl, heterocycloalkyl, aryl, or heteroaryl; wherein the cycloalkyl, heterocycloalkyl, aryl, and heteroaryl is optionally substituted with one or more R^{2a};
each R^{2a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{2a} on the same atom are taken together to form an oxo;
X is —N— or —CR³—;
Y is —O—, —NR⁴—, or —C(R⁵)₂—;
R³ is hydrogen, deuterium, halogen, —CN, —OH, —OR^{a}, —NR^{c}R^{d}, or C₁-C₆alkyl optionally substituted with one or more R;
R⁴ is hydrogen or C₁-C₆alkyl optionally substituted with one or more R;
each R⁵ is independently hydrogen, deuterium, halogen, —CN, —OH, —OR^{a}, —NR^{c}R^{d}, or C₁-C₆alkyl optionally substituted with one or more R;
or two R⁵ are taken together to form an oxo;
L is absent, —O—, —S—, —NR⁶—, —C(R⁷)₂—, —C(═O)—, —S(═O)—, —S(═O)₂—, —NR⁶—C(R⁷)₂—, —C(R⁷)₂—NR⁶—, —NR⁶—C(═O)—, —C(═O)—NR⁶—, or —NR⁶—C(═O)—NR⁶—;
R⁶ is hydrogen or C₁-C₆alkyl optionally substituted with one or more R;
each R⁷ is independently hydrogen, deuterium, halogen, —CN, —OH, —OR^{a}, —NR^{c}R^{d}, or C₁-C₆alkyl optionally substituted with one or more R;
or two R⁷ are taken together to form an oxo;
or two R⁷ are taken together to form a cycloalkyl or heterocycloalkyl; each optionally substituted with one or more R;
Z¹ is N or CR^{Z1};
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z2};
R^{Z2} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo;
provided that the compound is not:

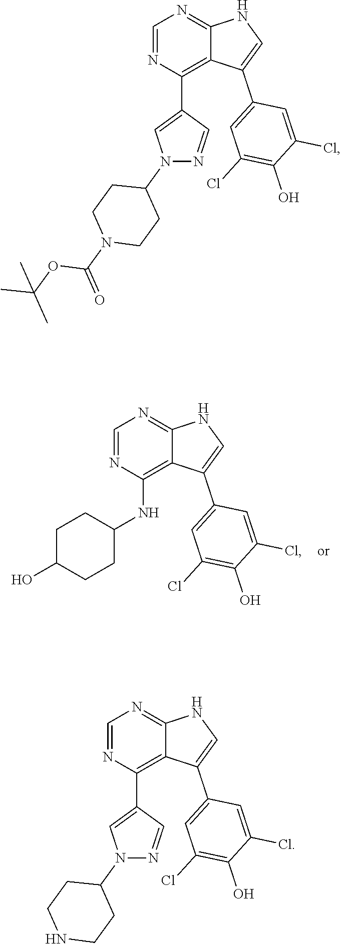 [SMILES: CC(C)(C)OC(=O)N1CCC(n2cc(-c3ncnc4[nH]cc(-c5cc(Cl)c(O)c(Cl)c5)c34)cn2)CC1 ; Oc1c(Cl)cc(-c2c[nH]c3ncnc(NC4CCC(O)CC4)c23)cc1Cl ; Oc1c(Cl)cc(-c2c[nH]c3ncnc(-c4cnn(C5CCNCC5)c4)c23)cc1Cl]

In some embodiments of a compound of Formula (II), Ring A is phenyl. In some embodiments of a compound of Formula (II), Ring A is 6-membered heteroaryl. In some embodiments of a compound of Formula (II), Ring A is pyridinyl, pyrimidinyl, or pyrazinyl. In some embodiments of a compound of Formula (II), Ring A is pyridinyl.

In some embodiments of a compound of Formula (II), the compound is of Formula (IIa):

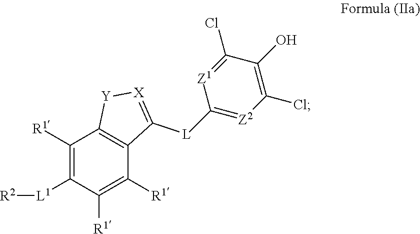

wherein each R^{1′} is independently hydrogen or R¹.

In some embodiments of a compound of Formula (II), the compound is of Formula (Ib):

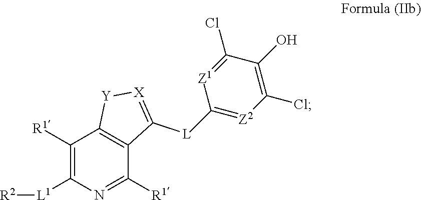

wherein each R^{1′} is independently hydrogen or R¹.

In some embodiments of a compound of Formula (II), (IIa), or (IIb), X is —N—. In some embodiments of a compound of Formula (II), (IIa), or (IIb), X is —CR³—.

In some embodiments of a compound of Formula (II), (IIa), or (IIb), Y is —O—. In some embodiments of a compound of Formula (II), (IIa), or (IIb), Y is —NR⁴—. In some embodiments of a compound of Formula (II), (IIa), or (IIb), Y is —C(R⁵)₂—.

In some embodiments of a compound of Formula (II), (IIa), or (IIb), R³ is hydrogen, deuterium, halogen, C₁-C₆alkyl, C₁-C₆haloalkyl, or C₁-C₆deuteroalkyl. In some embodiments of a compound of Formula (II), (IIa), or (IIb), R³ is hydrogen, C₁-C₆alkyl, or C₁-C₆haloalkyl. In some embodiments of a compound of Formula (II), (IIa), or (IIb), R³ is hydrogen.

In some embodiments of a compound of Formula (II), (IIa), or (IIb), R⁴ is hydrogen or C₁-C₆alkyl. In some embodiments of a compound of Formula (II), (IIa), or (IIb), R⁴ is hydrogen.

In some embodiments of a compound of Formula (II), (IIa), or (IIb), each R⁵ is independently hydrogen, deuterium, halogen, C₁-C₆alkyl, C₁-C₆haloalkyl, or C₁-C₆deuteroalkyl. In some embodiments of a compound of Formula (II), (IIa), or (IIb), each R⁵ is independently hydrogen or C₁-C₆alkyl. In some embodiments of a compound of Formula (II), (IIa), or (IIb), each R⁵ is hydrogen.

In some embodiments of a compound of Formula (II), (IIa), or (IIb), each R¹ is independently deuterium, halogen, C₁-C₆alkyl, or C₁-C₆haloalkyl. In some embodiments of a compound of Formula (II), (IIa), or (IIb), each R¹ is independently deuterium, halogen, or C₁-C₆alkyl. In some embodiments of a compound of Formula (II), (IIa), or (IIb), each R¹ is independently halogen or C₁-C₆alkyl.

In some embodiments of a compound of Formula (II), (IIa), or (IIb), n is 0-2. In some embodiments of a compound of Formula (II), (IIa), or (IIb), n is 0 or 1. In some embodiments of a compound of Formula (II), (IIa), or (IIb), n is 0. In some embodiments of a compound of Formula (II), (IIa), or (IIb), n is 1.

In some embodiments of a compound of Formula (II), (IIa), or (IIb), L¹ is absent. In some embodiments of a compound of Formula (II), (IIa), or (IIb), L¹ is —O—, —S—, or —NR⁶—. In some embodiments of a compound of Formula (II), (IIa), or (IIb), L¹ is —O—. In some embodiments of a compound of Formula (II), (IIa), or (IIb), L¹ is —S—. In some embodiments of a compound of Formula (II), (IIa), or (IIb), L¹ is —NR⁶—. In some embodiments of a compound of Formula (II), (IIa), or (IIb), L¹ is —C(═O)—, —C(═O)O—, —S(═O)—, or —S(═O)₂—. In some embodiments of a compound of Formula (II), (IIa), or (IIb), L¹ is —C(═O)—. In some embodiments of a compound of Formula (II), (IIa), or (IIb), L¹ is —C(═O)O—. In some embodiments of a compound of Formula (II), (IIa), or (IIb), L¹ is —S(═O)—. In some embodiments of a compound of Formula (II), (IIa), or (IIb), L¹ is —S(═O)₂—. In some embodiments of a compound of Formula (II), (IIa), or (IIb), L¹ is —NR⁶—C(R)₂— or —C(R⁷)₂—NR⁶—. In some embodiments of a compound of Formula (II), (IIa), or (IIb), L¹ is —NR⁶—C(R)₂—. In some embodiments of a compound of Formula (II), (IIa), or (IIb), L¹ is —C(R⁷)₂—NR⁶—. In some embodiments of a compound of Formula (H), (IIa), or (IIb), L¹ is —NR⁶—C(═O)— or —C(═O)—NR⁶—. In some embodiments of a compound of Formula (II), (IIa), or (IIb), L¹ is —NR⁶—C(═O)—. In some embodiments of a compound of Formula (II), (IIa), or (IIb), L¹ is —C(═O)—NR⁶—.

In some embodiments of a compound of Formula (II), (IIa), or (IIb), R² is cycloalkyl or heterocycloalkyl; wherein the cycloalkyl and heterocycloalkyl is optionally substituted with one or more R^{2a}. In some embodiments of a compound of Formula (II), (IIa), or (IIb), R² is heterocycloalkyl optionally substituted with one or more R^{2a}. In some embodiments of a compound of Formula (II), (IIa), or (IIb), R² is aryl or heteroaryl; wherein the aryl and heteroaryl is optionally substituted with one or more R^{2a}.

In some embodiments of a compound of Formula (II), (IIa), or (IIb), each R^{2a} is independently deuterium, halogen, —CN, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound of Formula (II), (IIa), or (IIb), each R^{2a} is independently deuterium, halogen, —CN, —OH, —OR^{a}, —S(═O)₂R^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl; wherein each alkyl is independently optionally substituted with one or more R. In some embodiments of a compound of Formula (II), (IIa), or (IIb), each R^{2a} is independently deuterium, halogen, —CN, —OH, —OR^{a}, —S(═O)₂R^{a}, —NR^{c}R^{d}, C₁-C₆alkyl, or C₁-C₆haloalkyl.

In some embodiments of a compound of Formula (II), (IIa), or (IIb), L is absent. In some embodiments of a compound of Formula (II), (IIa), or (IIb), L is —O—. In some embodiments of a compound of Formula (II), (IIa), or (IIb), L is —NR⁶—. In some embodiments of a compound of Formula (II), (IIa), or (IIb), L is —S—. In some embodiments of a compound of Formula (H), (IIa), or (IIb), L is —O—, —S—, or —NR⁶—. In some embodiments of a compound of Formula (II), (IIa), or (IIb), L is —C(R⁷)₂—. In some embodiments of a compound of Formula (II), (IIa), or (IIb), L is —C(═O)—, —S(═O)—, or —S(═O)₂—. In some embodiments of a compound of Formula (II), (IIa), or (IIb), L is —C(═O)—. In some embodiments of a compound of Formula (II), (IIa), or (IIb), L is —S(═O)—. In some embodiments of a compound of Formula (II), (IIa), or (IIb), L is —S(═O)₂—. In some embodiments of a compound of Formula (II), (IIa), or (IIb), L is —NR⁶—C(R⁷)₂— or —C(R⁷)₂—NR⁶—. In some embodiments of a compound of Formula (II), (IIa), or (IIb), L is —NR⁶—C(R⁷)₂—. In some embodiments of a compound of Formula (II), (IIa), or (IIb), L is —C(R⁷)₂—NR⁶—. In some embodiments of a compound of Formula (II), (IIa), or (IIb), L is —NR⁶—C(═O)— or —C(═O)—NR⁶—. In some embodiments of a compound of Formula (II), (IIa), or (IIb), L is —NR⁶—C(═O)—. In some embodiments of a compound of Formula (II), (IIa), or (IIb), L is —C(═O)—NR⁶—. In some embodiments of a compound of Formula (II), (IIa), or (IIb), L is —NR⁶—C(═O)—NR⁶—.

In some embodiments of a compound of Formula (II), (IIa), or (IIb), R⁶ is hydrogen or C₁-C₆alkyl. In some embodiments of a compound of Formula (II), (IIa), or (IIb), R⁶ is hydrogen.

In some embodiments of a compound of Formula (II), (IIa), or (IIb), each R⁷ is independently hydrogen, deuterium, halogen, C₁-C₆alkyl, C₁-C₆haloalkyl, or C₁-C₆deuteroalkyl. In some embodiments of a compound of Formula (II), (IIa), or (IIb), each R⁷ is independently hydrogen, halogen, or C₁-C₆alkyl.

In some embodiments of a compound of Formula (II), (IIa), or (IIb), Z¹ is N. In some embodiments of a compound of Formula (II), (IIa), or (IIb), Z¹ is CR^{Z1}.

In some embodiments of a compound of Formula (II), (IIa), or (IIb), R^{Z1} is hydrogen, deuterium, halogen, C₁-C₆alkyl, C₁-C₆haloalkyl, or C₁-C₆deuteroalkyl. In some embodiments of a compound of Formula (II), (IIa), or (IIb), R^{Z1} is hydrogen.

In some embodiments of a compound of Formula (II), (IIa), or (IIb), Z² is N. In some embodiments of a compound of Formula (II), (IIa), or (IIb), Z² is CR^{Z2}.

In some embodiments of a compound of Formula (II), (IIa), or (IIb), R^{Z2} is hydrogen, deuterium, halogen, C₁-C₆alkyl, C₁-C₆haloalkyl, or C₁-C₆deuteroalkyl. In some embodiments of a compound of Formula (II), (IIa), or (IIb), R^{Z2} is hydrogen.

Also disclosed herein is a compound of Formula (III), or a pharmaceutically acceptable salt, solvate, or stereoisomer thereof:

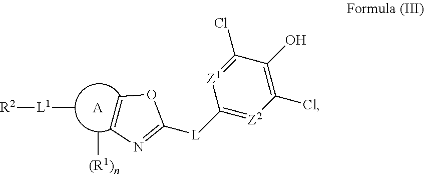

wherein:
Ring A is aryl or 6-membered heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
n is 0-3;
L¹ is absent, —O—, —S—, —NR⁶—, —C(═O)—, —C(═O)O—, —S(═O)—, —S(═O)₂—, —NR⁶—C(R⁷)₂—, —C(R⁷)₂—NR⁶—, —NR⁶—C(═O)—, or —C(═O)—NR⁶—;
R² is cycloalkyl, heterocycloalkyl, aryl, or heteroaryl; wherein the cycloalkyl, heterocycloalkyl, aryl, and heteroaryl is optionally substituted with one or more R^{2a};
each R^{2a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{2a} on the same atom are taken together to form an oxo;
L is absent, —O—, —S—, —NR⁶—, —C(R⁷)₂—, —C(═O)—, —S(═O)—, —S(═O)₂—, —NR⁶—C(R)₂—, —C(R⁷)₂—NR⁶—, —NR⁶—C(═O)—, —C(═O)—NR⁶—, or —NR⁶—C(═O)—NR⁶—;
R⁶ is hydrogen or C₁-C₆alkyl optionally substituted with one or more R;
each R⁷ is independently hydrogen, deuterium, halogen, —CN, —OH, —OR^{a}, —NR^{c}R^{d}, or C₁-C₆alkyl optionally substituted with one or more R;
or two R⁷ are taken together to form an oxo;
Z¹ is N or CR^{Z1};
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z2};
R^{Z2} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

In some embodiments of a compound of Formula (III), Ring A is phenyl. In some embodiments of a compound of Formula (III), Ring A is 6-membered heteroaryl. In some embodiments of a compound of Formula (III), Ring A is pyridinyl, pyrimidinyl, or pyrazinyl. In some embodiments of a compound of Formula (III), Ring A is pyridinyl.

In some embodiments of a compound of Formula (III), each R¹ is independently deuterium, halogen, C₁-C₆alkyl, or C₁-C₆haloalkyl. In some embodiments of a compound of Formula (III), each R¹ is independently deuterium, halogen, or C₁-C₆alkyl. In some embodiments of a compound of Formula (III), each R¹ is independently halogen or C₁-C₆alkyl.

In some embodiments of a compound of Formula (III), n is 0-2. In some embodiments of a compound of Formula (III), n is 0 or 1. In some embodiments of a compound of Formula (III), n is 0. In some embodiments of a compound of Formula (III), n is 1.

In some embodiments of a compound of Formula (III), L¹ is absent. In some embodiments of a compound of Formula (III), L¹ is —O—, —S—, or —NR⁶—. In some embodiments of a compound of Formula (III), L¹ is —O—. In some embodiments of a compound of Formula (III), L¹ is —S—. In some embodiments of a compound of Formula (III), L¹ is —NR⁶—. In some embodiments of a compound of Formula (III), L¹ is —C(═O)—, —C(═O)O—, —S(═O)—, or —S(═O)₂—. In some embodiments of a compound of Formula (III), L¹ is —C(═O)—. In some embodiments of a compound of Formula (III), L¹ is —C(═O)O—. In some embodiments of a compound of Formula (III), L¹ is —S(═O)—. In some embodiments of a compound of Formula (III), L¹ is —S(═O)₂—. In some embodiments of a compound of Formula (III), L¹ is —NR⁶—C(R⁷)₂— or —C(R⁷)₂—NR⁶—. In some embodiments of a compound of Formula (III), L¹ is —NR⁶—C(R⁷)₂—. In some embodiments of a compound of Formula (III), L¹ is —C(R⁷)₂—NR⁶—. In some embodiments of a compound of Formula (III), L¹ is —NR⁶—C(═O)— or —C(═O)—NR⁶—. In some embodiments of a compound of Formula (III), L¹ is —NR⁶—C(═O)—. In some embodiments of a compound of Formula (III), L¹ is —C(═O)—NR⁶—.

In some embodiments of a compound of Formula (III), R² is cycloalkyl or heterocycloalkyl; wherein the cycloalkyl and heterocycloalkyl is independently optionally substituted with one or more R^{2a}. In some embodiments of a compound of Formula (III), R² is heterocycloalkyl optionally substituted with one or more R^{2a}. In some embodiments of a compound of Formula (III), R² is aryl or heteroaryl; wherein the aryl and heteroaryl is independently optionally substituted with one or more R^{2a}.

In some embodiments of a compound of Formula (III), each R^{2a} is independently deuterium, halogen, —CN, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound of Formula (III), each R^{2a} is independently deuterium, halogen, —CN, —OH, —OR^{a}, —S(═O)₂R^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl; wherein each alkyl is independently optionally substituted with one or more R. In some embodiments of a compound of Formula (III), each R^{2a} is independently deuterium, halogen, —CN, —OH, —OR^{a}, —S(═O)₂R^{a}, —NR^{c}R^{d}, C₁-C₆alkyl, or C₁-C₆haloalkyl.

In some embodiments of a compound of Formula (III), L is absent. In some embodiments of a compound of Formula (III), L is —O—. In some embodiments of a compound of Formula (III), L is —NR⁶—. In some embodiments of a compound of Formula (III), L is —S—. In some embodiments of a compound of Formula (III), L is —O—, —S—, or —NR⁶—. In some embodiments of a compound of Formula (III), L is —C(R⁷)₂—. In some embodiments of a compound of Formula (III), L is —C(═O)—, —S(═O)—, or —S(═O)₂—. In some embodiments of a compound of Formula (III), L is —C(═O)—. In some embodiments of a compound of Formula (III), L is —S(═O)—. In some embodiments of a compound of Formula (III), L is —S(═O)₂—. In some embodiments of a compound of Formula (III), L is —NR⁶—C(R⁷)₂— or —C(R⁷)₂—NR⁶—. In some embodiments of a compound of Formula (III), L is —NR⁶—C(R⁷)₂—. In some embodiments of a compound of Formula (III), L is —C(R⁷)₂—NR⁶—. In some embodiments of a compound of Formula (III), L is —NR⁶—C(═O)— or —C(═O)—NR⁶—. In some embodiments of a compound of Formula (III), L is —NR⁶—C(═O)—. In some embodiments of a compound of Formula (III), L is —C(═O)—NR⁶—. In some embodiments of a compound of Formula (III), L is —NR⁶—C(═O)—NR⁶—.

In some embodiments of a compound of Formula (III), R⁶ is hydrogen or C₁-C₆alkyl. In some embodiments of a compound of Formula (III), R⁶ is hydrogen.

In some embodiments of a compound of Formula (III), each R⁷ is independently hydrogen, deuterium, halogen, C₁-C₆alkyl, C₁-C₆haloalkyl, or C₁-C₆deuteroalkyl. In some embodiments of a compound of Formula (III), each R⁷ is independently hydrogen, halogen, or C₁-C₆alkyl.

In some embodiments of a compound of Formula (III), Z¹ is N. In some embodiments of a compound of Formula (III), Z¹ is CR^{Z1}.

In some embodiments of a compound of Formula (III), R^{Z1} is hydrogen, deuterium, halogen, C₁-C₆alkyl, C₁-C₆haloalkyl, or C₁-C₆deuteroalkyl. In some embodiments of a compound of Formula (III), R^{Z1} is hydrogen.

In some embodiments of a compound of Formula (III), Z² is N. In some embodiments of a compound of Formula (III), Z² is CR^{Z2}.

In some embodiments of a compound of Formula (III), R^{Z2} is hydrogen, deuterium, halogen, C₁-C₆alkyl, C₁-C₆haloalkyl, or C₁-C₆deuteroalkyl. In some embodiments of a compound of Formula (III), R^{Z2} is hydrogen.

Also disclosed herein is a compound of Formula (IV), or a pharmaceutically acceptable salt, solvate, or stereoisomer thereof:

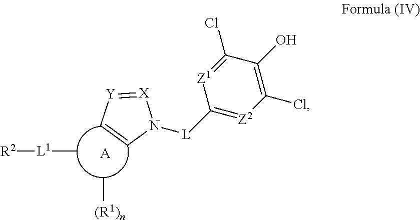

wherein:
Ring A is phenyl or 6-membered heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
n is 0-3;
L¹ is absent, —O—, —S—, —NR⁶—, —C(═O)—, —C(═O)O—, —S(═O)—, —S(═O)₂—, —NR⁶—C(R⁷)₂—, —C(R⁷)₂—NR⁶—, —NR⁶—C(═O)—, or —C(═O)—NR⁶—;
R² is cycloalkyl, heterocycloalkyl, aryl, or heteroaryl; wherein the cycloalkyl, heterocycloalkyl, aryl, and heteroaryl is optionally substituted with one or more R^{2a};
each R^{2a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{2a} on the same atom are taken together to form an oxo;
X is —N— or —CR^{X}—;
R^{X} is hydrogen, deuterium, halogen, —CN, —OH, —OR^{a}, —NR^{c}R^{d}, or C₁-C₆alkyl optionally substituted with one or more R;
Y is —N— or —CR^{Y}—;
R^{Y} is hydrogen, deuterium, halogen, —CN, —OH, —OR^{a}, —NR^{c}R^{d}, or C₁-C₆alkyl optionally substituted with one or more R;
L is absent, —C(R⁷)₂—, —C(═O)—, —S(═O)—, —S(═O)₂—, —C(R)₂—NR⁶—, or —C(═O)—NR⁶—;
R⁶ is hydrogen or C₁-C₆alkyl optionally substituted with one or more R;
each R⁷ is independently hydrogen, deuterium, halogen, —CN, —OH, —OR^{a}, —NR^{c}R^{d}, or C₁-C₆alkyl optionally substituted with one or more R;
or two R⁷ are taken together to form an oxo;
or two R⁷ are taken together to form a cycloalkyl or heterocycloalkyl; each optionally substituted with one or more R;
Z¹ is N or CR^{Z}1;
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z2};
R^{Z2} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

In some embodiments of a compound of Formula (IV), Ring A is phenyl. In some embodiments of a compound of Formula (IV), Ring A is 6-membered heteroaryl. In some embodiments of a compound of Formula (IV), Ring A is pyridinyl, pyrimidinyl, or pyrazinyl. In some embodiments of a compound of Formula (IV), Ring A is pyridinyl.

In some embodiments of a compound of Formula (IV), the compound is of Formula (IVa):

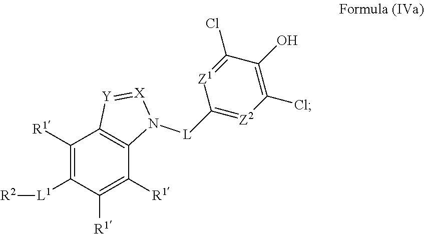

wherein each R^{1′} is independently hydrogen or R¹.

In some embodiments of a compound of Formula (IV), the compound is of Formula (IVb):

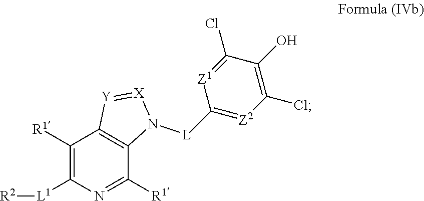

wherein each R^{1′} is independently hydrogen or R¹.

In some embodiments of a compound of Formula (IV), (IVa), or (IVb), X is —N—. In some embodiments of a compound of Formula (IV), (IVa), or (IVb), X is —CR^{X}—.

In some embodiments of a compound of Formula (IV), (IVa), or (IVb), R^{X} is hydrogen, deuterium, halogen, C₁-C₆alkyl, C₁-C₆haloalkyl, or C₁-C₆deuteroalkyl. In some embodiments of a compound of Formula (IV), (IVa), or (IVb), R^{X} is hydrogen, C₁-C₆alkyl, or C₁-C₆haloalkyl. In some embodiments of a compound of Formula (IV), (IVa), or (IVb), R^{X} is hydrogen.

In some embodiments of a compound of Formula (IV), (IVa), or (IVb), Y is —N—. In some embodiments of a compound of Formula (IV), (IVa), or (IVb), Y is —CR^{Y}—.

In some embodiments of a compound of Formula (IV), (IVa), or (IVb), R^{Y} is hydrogen, deuterium, halogen, C₁-C₆alkyl, C₁-C₆haloalkyl, or C₁-C₆deuteroalkyl. In some embodiments of a compound of Formula (IV), (IVa), or (IVb), R^{Y} is hydrogen, C₁-C₆alkyl, or C₁-C₆haloalkyl. In some embodiments of a compound of Formula (IV), (IVa), or (IVb), R^{Y} is hydrogen.

In some embodiments of a compound of Formula (IV), (IVa), or (IVb), each R¹ is independently deuterium, halogen, C₁-C₆alkyl, or C₁-C₆haloalkyl. In some embodiments of a compound of Formula (IV), (IVa), or (IVb), each R¹ is independently deuterium, halogen, or C₁-C₆alkyl. In some embodiments of a compound of Formula (IV), (IVa), or (IVb), each R¹ is independently halogen or C₁-C₆alkyl.

In some embodiments of a compound of Formula (IV), (IVa), or (IVb), n is 0-2. In some embodiments of a compound of Formula (IV), (IVa), or (IVb), n is 0 or 1. In some embodiments of a compound of Formula (IV), (IVa), or (IVb), n is 0. In some embodiments of a compound of Formula (IV), (IVa), or (IVb), n is 1.

In some embodiments of a compound of Formula (IV), (IVa), or (IVb), L¹ is absent. In some embodiments of a compound of Formula (IV), (IVa), or (IVb), L¹ is —O—, —S—, or —NR⁶—. In some embodiments of a compound of Formula (IV), (IVa), or (IVb), L¹ is —O—. In some embodiments of a compound of Formula (IV), (IVa), or (IVb), L¹ is —S—. In some embodiments of a compound of Formula (IV), (IVa), or (IVb), L¹ is —NR⁶—. In some embodiments of a compound of Formula (IV), (IVa), or (IVb), L¹ is —C(═O)—, —C(═O)O—, —S(═O)—, or —S(═O)₂—. In some embodiments of a compound of Formula (IV), (IVa), or (IVb), L¹ is —C(═O)—. In some embodiments of a compound of Formula (IV), (IVa), or (IVb), L¹ is —C(═O)O—. In some embodiments of a compound of Formula (IV), (IVa), or (IVb), L¹ is —S(═O)—. In some embodiments of a compound of Formula (IV), (IVa), or (IVb), L¹ is —S(═O)₂—. In some embodiments of a compound of Formula (IV), (IVa), or (IVb), L¹ is —NR⁶—C(R⁷)₂— or —C(R⁷)₂—NR⁶—. In some embodiments of a compound of Formula (IV), (IVa), or (IVb), L¹ is —NR⁶—C(R⁷)₂—. In some embodiments of a compound of Formula (IV), (IVa), or (IVb), L¹ is —C(R⁷)₂—NR⁶—. In some embodiments of a compound of Formula (IV), (IVa), or (IVb), L¹ is —NR⁶—C(═O)— or —C(═O)—NR⁶—. In some embodiments of a compound of Formula (IV), (IVa), or (IVb), L¹ is —NR⁶—C(═O)—. In some embodiments of a compound of Formula (IV), (IVa), or (IVb), L¹ is —C(═O)—NR⁶—.

In some embodiments of a compound of Formula (IV), (IVa), or (IVb), R² is cycloalkyl or heterocycloalkyl; wherein the cycloalkyl and heterocycloalkyl is optionally substituted with one or more R^{2a}. In some embodiments of a compound of Formula (IV), (IVa), or (IVb), R² is heterocycloalkyl optionally substituted with one or more R^{2a}. In some embodiments of a compound of Formula (IV), (IVa), or (IVb), R² is aryl or heteroaryl; wherein the aryl and heteroaryl is optionally substituted with one or more R^{2a}.

In some embodiments of a compound of Formula (IV), (IVa), or (IVb), each R^{2a} is independently deuterium, halogen, —CN, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound of Formula (IV), (IVa), or (IVb), each R^{2a} is independently deuterium, halogen, —CN, —OH, —OR^{a}, —S(═O)₂R^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl; wherein each alkyl is independently optionally substituted with one or more R. In some embodiments of a compound of Formula (IV), (IVa), or (IVb), each R^{2a} is independently deuterium, halogen, —CN, —OH, —OR^{a}, —S(═O)₂R^{a}, —NR^{c}R^{d}, C₁-C₆alkyl, or C₁-C₆haloalkyl.

In some embodiments of a compound of Formula (IV), (IVa), or (IVb), L is absent. In some embodiments of a compound of Formula (IV), (IVa), or (IVb), L is —C(R⁷)₂—. In some embodiments of a compound of Formula (IV), (IVa), or (IVb), L is —C(═O)—, —S(═O)—, or —S(═O)₂—. In some embodiments of a compound of Formula (IV), (IVa), or (IVb), L is —C(═O)—. In some embodiments of a compound of Formula (IV), (IVa), or (IVb), L is —S(═O)—. In some embodiments of a compound of Formula (IV), (IVa), or (IVb), L is —S(═O)₂—. In some embodiments of a compound of Formula (IV), (IVa), or (IVb), L is —C(R⁷)₂—NR⁶—. In some embodiments of a compound of Formula (IV), (IVa), or (IVb), L is —C(═O)— NR⁶—.

In some embodiments of a compound of Formula (IV), (IVa), or (IVb), R⁶ is hydrogen or C₁-C₆alkyl. In some embodiments of a compound of Formula (IV), (IVa), or (IVb), R⁶ is hydrogen.

In some embodiments of a compound of Formula (IV), (IVa), or (IVb), each R⁷ is independently hydrogen, deuterium, halogen, C₁-C₆alkyl, C₁-C₆haloalkyl, or C₁-C₆deuteroalkyl. In some embodiments of a compound of Formula (IV), (IVa), or (IVb), each R⁷ is independently hydrogen, halogen, or C₁-C₆alkyl.

In some embodiments of a compound of Formula (IV), (IVa), or (IVb), Z¹ is N. In some embodiments of a compound of Formula (IV), (IVa), or (IVb), Z¹ is CR^{Z1}.

In some embodiments of a compound of Formula (IV), (IVa), or (IVb), R^{Z1} is hydrogen, deuterium, halogen, C₁-C₆alkyl, C₁-C₆haloalkyl, or C₁-C₆deuteroalkyl. In some embodiments of a compound of Formula (IV), (IVa), or (IVb), R^{Z1} is hydrogen.

In some embodiments of a compound of Formula (IV), (IVa), or (IVb), Z² is N. In some embodiments of a compound of Formula (IV), (IVa), or (IVb), Z² is CR^{Z2}.

In some embodiments of a compound of Formula (IV), (IVa), or (IVb), R^{Z2} is hydrogen, deuterium, halogen, C₁-C₆alkyl, C₁-C₆haloalkyl, or C₁-C₆deuteroalkyl. In some embodiments of a compound of Formula (IV), (IVa), or (IVb), R^{Z2} is hydrogen.

Also disclosed herein is a compound of Formula (V), or a pharmaceutically acceptable salt, solvate, or stereoisomer thereof:

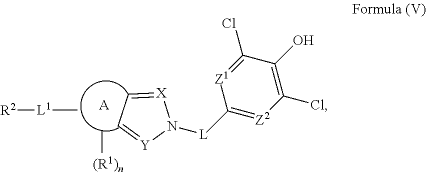

wherein:
Ring A is phenyl or 6-membered heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
n is 0-3;
L¹ is absent, —O—, —S—, —NR⁶—, —C(═O)—, —C(═O)O—, —S(═O)—, —S(═O)₂—, —NR⁶—C(R⁷)₂—, —C(R⁷)₂—NR⁶—, —NR⁶—C(═O)—, or —C(═O)—NR⁶—;
R² is cycloalkyl, heterocycloalkyl, aryl, or heteroaryl; wherein the cycloalkyl, heterocycloalkyl, aryl, and heteroaryl is optionally substituted with one or more R^{2a};
each R^{2a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{2a} on the same atom are taken together to form an oxo;
X is —N— or —CR^{X}—;
R^{X} is hydrogen, deuterium, halogen, —CN, —OH, —OR^{a}, —NR^{c}R^{d}, or C₁-C₆alkyl optionally substituted with one or more R;
Y is —N— or —CR^{Y}—;
R^{Y} is hydrogen, deuterium, halogen, —CN, —OH, —OR^{a}, —NR^{c}R^{d}, or C₁-C₆alkyl optionally substituted with one or more R;
L is absent, —C(R⁷)₂—, —C(═O)—, —S(═O)—, —S(═O)₂—, —C(R⁷)₂—NR⁶—, or —C(═O)—NR⁶—;
R⁶ is hydrogen or C₁-C₆alkyl optionally substituted with one or more R;
each R⁷ is independently hydrogen, deuterium, halogen, —CN, —OH, —OR^{a}, —NR^{c}R^{d}, or C₁-C₆alkyl optionally substituted with one or more R;
or two R⁷ are taken together to form an oxo;
or two R⁷ are taken together to form a cycloalkyl or heterocycloalkyl; each optionally substituted with one or more R;
Z¹ is N or CR^{Z1};
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z2};
R^{Z2} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

In some embodiments of a compound of Formula (V), Ring A is phenyl. In some embodiments of a compound of Formula (V), Ring A is 6-membered heteroaryl. In some embodiments of a compound of Formula (V), Ring A is pyridinyl, pyrimidinyl, or pyrazinyl. In some embodiments of a compound of Formula (V), Ring A is pyridinyl.

In some embodiments of a compound of Formula (V), the compound is of Formula (Va):

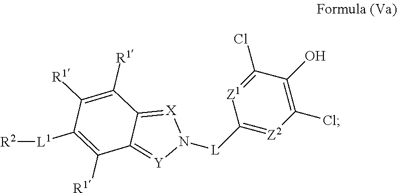

wherein each R^{1′} is independently hydrogen or R¹.

In some embodiments of a compound of Formula (V), the compound is of Formula (Vb):

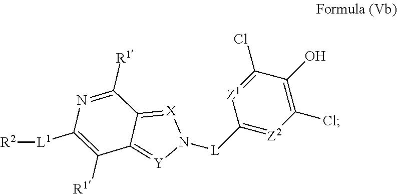

wherein each R^{1′} is independently hydrogen or R¹.

In some embodiments of a compound of Formula (V), (Va), or (Vb), X is —N—. In some embodiments of a compound of Formula (V), (Va), or (Vb), X is —CR^{X}—.

In some embodiments of a compound of Formula (V), (Va), or (Vb), R^{X} is hydrogen, deuterium, halogen, C₁-C₆alkyl, C₁-C₆haloalkyl, or C₁-C₆deuteroalkyl. In some embodiments of a compound of Formula (V), (Va), or (Vb), R^{X} is hydrogen, C₁-C₆alkyl, or C₁-C₆haloalkyl. In some embodiments of a compound of Formula (V), (Va), or (Vb), R^{X} is hydrogen.

In some embodiments of a compound of Formula (V), (Va), or (Vb), Y is —N—. In some embodiments of a compound of Formula (V), (Va), or (Vb), Y is —CR^{Y}—.

In some embodiments of a compound of Formula (V), (Va), or (Vb), R^{Y} is hydrogen, deuterium, halogen, C₁-C₆alkyl, C₁-C₆haloalkyl, or C₁-C₆deuteroalkyl. In some embodiments of a compound of Formula (V), (Va), or (Vb), R^{Y} is hydrogen, C₁-C₆alkyl, or C₁-C₆haloalkyl. In some embodiments of a compound of Formula (V), (Va), or (Vb), R^{Y} is hydrogen.

In some embodiments of a compound of Formula (V), (Va), or (Vb), each R¹ is independently deuterium, halogen, C₁-C₆alkyl, or C₁-C₆haloalkyl. In some embodiments of a compound of Formula (V), (Va), or (Vb), each R¹ is independently deuterium, halogen, or C₁-C₆alkyl. In some embodiments of a compound of Formula (V), (Va), or (Vb), each R¹ is independently halogen or C₁-C₆alkyl.

In some embodiments of a compound of Formula (V), (Va), or (Vb), n is 0-2. In some embodiments of a compound of Formula (V), (Va), or (Vb), n is 0 or 1. In some embodiments of a compound of Formula (V), (Va), or (Vb), n is 0. In some embodiments of a compound of Formula (V), (Va), or (Vb), n is 1.

In some embodiments of a compound of Formula (V), (Va), or (Vb), L¹ is absent. In some embodiments of a compound of Formula (V), (Va), or (Vb), L¹ is —O—, —S—, or —NR⁶—. In some embodiments of a compound of Formula (V), (Va), or (Vb), L¹ is —O—. In some embodiments of a compound of Formula (V), (Va), or (Vb), L¹ is —S—. In some embodiments of a compound of Formula (V), (Va), or (Vb), L¹ is —NR⁶—. In some embodiments of a compound of Formula (V), (Va), or (Vb), L¹ is —C(═O)—, —C(═O)O—, —S(═O)—, or —S(═O)₂—. In some embodiments of a compound of Formula (V), (Va), or (Vb), L¹ is —C(═O)—. In some embodiments of a compound of Formula (V), (Va), or (Vb), L¹ is —C(═O)O—. In some embodiments of a compound of Formula (V), (Va), or (Vb), L¹ is —S(═O)—. In some embodiments of a compound of Formula (V), (Va), or (Vb), L¹ is —S(═O)₂—. In some embodiments of a compound of Formula (V), (Va), or (Vb), L¹ is —NR⁶—C(R⁷)₂— or —C(R⁷)₂—NR⁶—. In some embodiments of a compound of Formula (V), (Va), or (Vb), L¹ is —NR⁶—C(R⁷)₂—. In some embodiments of a compound of Formula (V), (Va), or (Vb), L¹ is —C(R⁷)₂—NR⁶—. In some embodiments of a compound of Formula (V), (Va), or (Vb), L¹ is —NR⁶—C(═O)— or —C(═O)—NR⁶—. In some embodiments of a compound of Formula (V), (Va), or (Vb), L¹ is —NR⁶—C(═O)—. In some embodiments of a compound of Formula (V), (Va), or (Vb), L¹ is —C(═O)—NR⁶—.

In some embodiments of a compound of Formula (V), (Va), or (Vb), R² is cycloalkyl or heterocycloalkyl; wherein the cycloalkyl and heterocycloalkyl is optionally substituted with one or more R^{2a}. In some embodiments of a compound of Formula (V), (Va), or (Vb), R² is heterocycloalkyl optionally substituted with one or more R^{2a}. In some embodiments of a compound of Formula (V), (Va), or (Vb), R² is aryl or heteroaryl; wherein the aryl and heteroaryl is optionally substituted with one or more R^{2a}.

In some embodiments of a compound of Formula (V), (Va), or (Vb), each R^{2a} is independently deuterium, halogen, —CN, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound of Formula (V), (Va), or (Vb), each R^{2a} is independently deuterium, halogen, —CN, —OH, —OR^{a}, —S(═O)₂R^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl; wherein each alkyl is independently optionally substituted with one or more R. In some embodiments of a compound of Formula (V), (Va), or (Vb), each R^{2a} is independently deuterium, halogen, —CN, —OH, —OR^{a}, —S(═O)₂R^{a}, —NR^{c}R^{d}, C₁-C₆alkyl, or C₁-C₆haloalkyl.

In some embodiments of a compound of Formula (V), (Va), or (Vb), L is absent. In some embodiments of a compound of Formula (V), (Va), or (Vb), L is —C(R⁷)₂—. In some embodiments of a compound of Formula (V), (Va), or (Vb), L is —C(═O)—, —S(═O)—, or —S(═O)₂—. In some embodiments of a compound of Formula (V), (Va), or (Vb), L is —C(═O)—. In some embodiments of a compound of Formula (V), (Va), or (Vb), L is —S(═O)—. In some embodiments of a compound of Formula (V), (Va), or (Vb), L is —S(═O)₂—. In some embodiments of a compound of Formula (V), (Va), or (Vb), L is —C(R⁷)₂—NR⁶—. In some embodiments of a compound of Formula (V), (Va), or (Vb), L is —C(═O)—NR⁶—.

In some embodiments of a compound of Formula (V), (Va), or (Vb), R⁶ is hydrogen or C₁-C₆alkyl. In some embodiments of a compound of Formula (V), (Va), or (Vb), R⁶ is hydrogen.

In some embodiments of a compound of Formula (V), (Va), or (Vb), each R⁷ is independently hydrogen, deuterium, halogen, C₁-C₆alkyl, C₁-C₆haloalkyl, or C₁-C₆deuteroalkyl. In some embodiments of a compound of Formula (V), (Va), or (Vb), each R⁷ is independently hydrogen, halogen, or C₁-C₆alkyl.

In some embodiments of a compound of Formula (V), (Va), or (Vb), Z¹ is N. In some embodiments of a compound of Formula (V), (Va), or (Vb), Z¹ is CR^{Z1}.

In some embodiments of a compound of Formula (V), (Va), or (Vb), R^{Z1} is hydrogen, deuterium, halogen, C₁-C₆alkyl, C₁-C₆haloalkyl, or C₁-C₆deuteroalkyl. In some embodiments of a compound of Formula (V), (Va), or (Vb), R^{Z1} is hydrogen.

In some embodiments of a compound of Formula (V), (Va), or (Vb), Z² is N. In some embodiments of a compound of Formula (V), (Va), or (Vb), Z² is CR^{Z2}.

In some embodiments of a compound of Formula (V), (Va), or (Vb), R^{Z2} is hydrogen, deuterium, halogen, C₁-C₆alkyl, C₁-C₆haloalkyl, or C₁-C₆deuteroalkyl. In some embodiments of a compound of Formula (V), (Va), or (Vb), R^{Z2} is hydrogen.

Also disclosed herein is a compound of Formula (VI), or a pharmaceutically acceptable salt, solvate, or stereoisomer thereof:

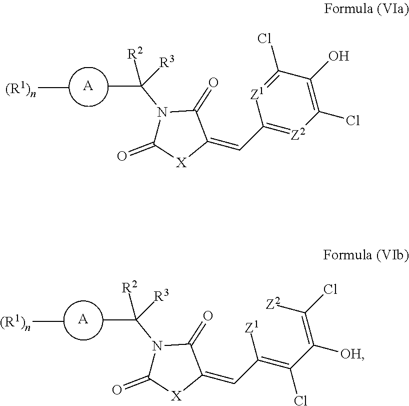

wherein:
Ring A is cycloalkyl, heterocycloalkyl, aryl, or heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or 2 R¹ on the same atoms are taken together to form an oxo;
n is 0-4;
R² is hydrogen, deuterium, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
R³ is hydrogen, deuterium, or C₁-C₆alkyl;
X is —O— or —S—;
Z¹ is N or CR^{Z1};
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z2};
R^{Z2} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

In some embodiments of a compound of Formula (VI), the compound is of Formula (VIa):

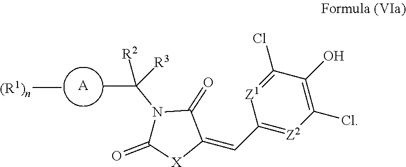

In some embodiments of a compound of Formula (VI), the compound is of Formula (VIb):

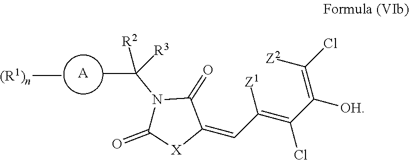

In some embodiments of a compound of Formula (VI), (VIa), or (VIb), Ring A is aryl or heteroaryl. In some embodiments of a compound of Formula (VI), (VIa), or (VIb), Ring A is phenyl. In some embodiments of a compound of Formula (VI), (VIa), or (VIb), Ring A is 5- or 6-membered heteroaryl. In some embodiments of a compound of Formula (VI), (VIa), or (VIb), Ring A is pyridinyl, pyrimidinyl, or pyrazinyl. In some embodiments of a compound of Formula (VI), (VIa), or (VIb), Ring A is pyridinyl. In some embodiments of a compound of Formula (VI), (VIa), or (VIb), Ring A is cycloalkyl or heterocycloalkyl.

In some embodiments of a compound of Formula (VI), (VIa), or (VIb), each R¹ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R.

In some embodiments of a compound of Formula (VI), (VIa), or (VIb), each R¹ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound of Formula (VI), (VIa), or (VIb), each R¹ is independently deuterium, halogen, —CN, —OH, —OR^{a}, —NR^{c}R^{d}, C₁-C₆alkyl, C₁-C₆haloalkyl, or C₁-C₆deuteroalkyl. In some embodiments of a compound of Formula (VI), (VIa), or (VIb), each R¹ is independently deuterium, halogen, —CN, —OH, —OR^{a}, C₁-C₆alkyl, or C₁-C₆haloalkyl.

In some embodiments of a compound of Formula (VI), (VIa), or (VIb), n is 0-2. In some embodiments of a compound of Formula (VI), (VIa), or (VIb), n is 0 or 1. In some embodiments of a compound of Formula (VI), (VIa), or (VIb), n is 1. In some embodiments of a compound of Formula (VI), (VIa), or (VIb), n is 0.

In some embodiments of a compound of Formula (VI), (VIa), or (VIb), R² is hydrogen, deuterium, or C₁-C₆alkyl. In some embodiments of a compound of Formula (VI), (VIa), or (VIb), R² is hydrogen.

In some embodiments of a compound of Formula (VI), (VIa), or (VIb), R³ is hydrogen.

In some embodiments of a compound of Formula (VI), (VIa), or (VIb), X is —O—. In some embodiments of a compound of Formula (VI), (VIa), or (VIb), X is —S—.

In some embodiments of a compound of Formula (VI), (VIa), or (VIb), Z¹ is N. In some embodiments of a compound of Formula (VI), (VIa), or (VIb), Z¹ is CR^{Z1}.

In some embodiments of a compound of Formula (VI), (VIa), or (VIb), R^{Z1} is hydrogen, deuterium, halogen, C₁-C₆alkyl, C₁-C₆haloalkyl, or C₁-C₆deuteroalkyl. In some embodiments of a compound of Formula (VI), (VIa), or (VIb), R^{Z1} is hydrogen.

In some embodiments of a compound of Formula (VI), (VIa), or (VIb), Z² is N. In some embodiments of a compound of Formula (VI), (VIa), or (VIb), Z² is CR^{Z2}.

In some embodiments of a compound of Formula (VI), (VIa), or (VIb), R^{Z2} is hydrogen, deuterium, halogen, C₁-C₆alkyl, C₁-C₆haloalkyl, or C₁-C₆deuteroalkyl. In some embodiments of a compound of Formula (VI), (VIa), or (VIb), R^{Z2} is hydrogen.

Also disclosed herein is a compound of Formula (VII), or a pharmaceutically acceptable salt, solvate, or stereoisomer thereof:

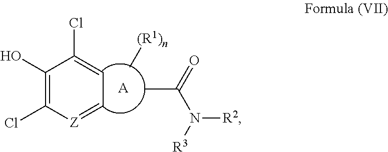

wherein:
Ring A is cycloalkyl, heterocycloalkyl, aryl, or heteroaryl;
each R¹ is independently deuterium, halogen, —CN, —NO₂, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R¹ on the same atom are taken together to form an oxo;
n is 0-4;
R² is hydrogen or C₁-C₆alkyl;
R³ is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, alkenyl, alkynyl, cycloalkyl, heterocycloalkyl, aryl, and heteroaryl is optionally substituted with one or more R^{3a};
each R^{3a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{3a} on the same atom are taken together to form an oxo;
Z is N or CR^{Z};
R^{Z} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

In some embodiments of a compound of Formula (VII), Ring A is 6-membered cycloalkyl, 6-membered heterocycloalkyl, phenyl, or 6-membered heteroaryl. In some embodiments of a compound of Formula (VII), Ring A is 6-membered cycloalkyl or 6-membered heterocycloalkyl. In some embodiments of a compound of Formula (VII), Ring A is phenyl or 6-membered heteroaryl. In some embodiments of a compound of Formula (VII), Ring A is phenyl. In some embodiments of a compound of Formula (VII), Ring A is 6-membered heteroaryl.

In some embodiments of a compound of Formula (VII), each R¹ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound of Formula (VII), each R¹ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl; wherein each alkyl is independently optionally substituted with one or more R. In some embodiments of a compound of Formula (VII), each R¹ is independently deuterium, halogen, —CN, —OH, —OR^{a}, —NR^{c}R^{d}, C₁-C₆alkyl, C₁-C₆haloalkyl, or C₁-C₆deuteroalkyl.

In some embodiments of a compound of Formula (VII), each R¹ is independently halogen, C₁-C₆alkyl, or C₁-C₆haloalkyl.

In some embodiments of a compound of Formula (VII), n is 0-2. In some embodiments of a compound of Formula (VII), n is 0 or 1. In some embodiments of a compound of Formula (VII), n is 0. In some embodiments of a compound of Formula (VII), n is 1.

In some embodiments of a compound of Formula (VII), R² is hydrogen.

In some embodiments of a compound of Formula (VII), R³ is hydrogen, C₁-C₆alkyl, C₁-C₆haloalkyl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, cycloalkyl, heterocycloalkyl, aryl, and heteroaryl is optionally substituted with one or more R^{3a}. In some embodiments of a compound of Formula (VII), R³ is C₁-C₆alkylene(cycloalkyl), C₁-C₆alkylene(heterocycloalkyl), C₁-C₆alkylene(aryl), or C₁-C₆alkylene(heteroaryl); wherein the alkylene, cycloalkyl, heterocycloalkyl, aryl, and heteroaryl is optionally substituted with one or more R^{3a}. In some embodiments of a compound of Formula (VII), R³ is C₁-C₆alkylene(aryl) or C₁-C₆alkylene(heteroaryl); wherein the alkylene, aryl, and heteroaryl is optionally substituted with one or more R^{3a}. In some embodiments of a compound of Formula (VII), R³ is C₁-C₆alkylene(aryl); wherein the alkylene and aryl is optionally substituted with one or more R^{3a}.

In some embodiments of a compound of Formula (VII), each R^{3a} is independently deuterium, halogen, —CN, —OH, —OR^{a}, —S(═O)R^{a}, —S(═O)₂R^{a}, —S(═O)₂NR^{c}R^{d}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl; wherein each alkyl is independently optionally substituted with one or more R. In some embodiments of a compound of Formula (VII), each R^{3a} is independently deuterium, halogen, —CN, —OH, —OR^{a}, —NR^{c}R^{d}, C₁-C₆alkyl, C₁-C₆haloalkyl, or C₁-C₆deuteroalkyl; wherein each alkyl is independently optionally substituted with one or more R. In some embodiments of a compound of Formula (VII), each R^{3a} is independently deuterium, halogen, —CN, —OH, —OR^{a}, —NR^{c}R^{d}, C₁-C₆alkyl, or C₁-C₆haloalkyl.

In some embodiments of a compound of Formula (VII), Z is N. In some embodiments of a compound of Formula (VII), Z is CR^{Z}.

In some embodiments of a compound of Formula (VII), R^{Z} is hydrogen, deuterium, halogen, C₁-C₆alkyl, C₁-C₆haloalkyl, or C₁-C₆deuteroalkyl. In some embodiments of a compound of Formula (VII), R^{Z} is hydrogen.

Also disclosed herein is a compound of Formula (VIIIa) or (VIIIb), or a pharmaceutically acceptable salt, solvate, or stereoisomer thereof:

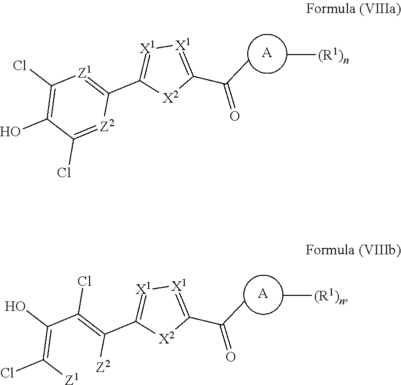

wherein:
Ring A is cycloalkyl, heterocycloalkyl, aryl, or heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R¹ on the same atom are taken together to form an oxo;
n is 0-4;
Z¹ is N or CR^{Z}1;
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z}1;
R^{Z2} is hydrogen, deuterium, halogen, —CN, —OH, —OR^{a}, —NR^{c}R^{d}, or C₁-C₆alkyl optionally substituted with one or more R;
each X¹ is independently —N— or —CR^{X1}—;
each R^{X1} is hydrogen, deuterium, halogen, —CN, —OH, —OR^{a}, —NR^{c}R^{d}, or C₁-C₆alkyl optionally substituted with one or more R;
or two R^{X1} are taken together to form a cycloalkyl, heterocycloalkyl, aryl, or heteroaryl; each optionally substituted with one or more R; X² is —O—, —S—, or —NR^{X2}—;
R^{X2} is hydrogen or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo;
provided that the compound is not:

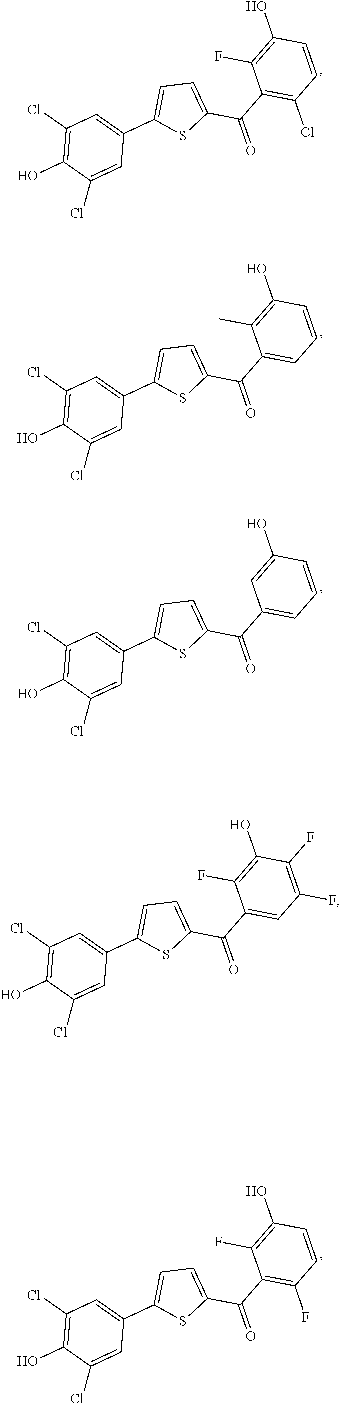 [SMILES: O=C(c1ccc(-c2cc(Cl)c(O)c(Cl)c2)s1)c1c(Cl)ccc(O)c1F ; Cc1c(O)cccc1C(=O)c1ccc(-c2cc(Cl)c(O)c(Cl)c2)s1 ; O=C(c1cccc(O)c1)c1ccc(-c2cc(Cl)c(O)c(Cl)c2)s1 ; O=C(c1ccc(-c2cc(Cl)c(O)c(Cl)c2)s1)c1cc(F)c(F)c(O)c1F ; O=C(c1ccc(-c2cc(Cl)c(O)c(Cl)c2)s1)c1c(F)ccc(O)c1F]

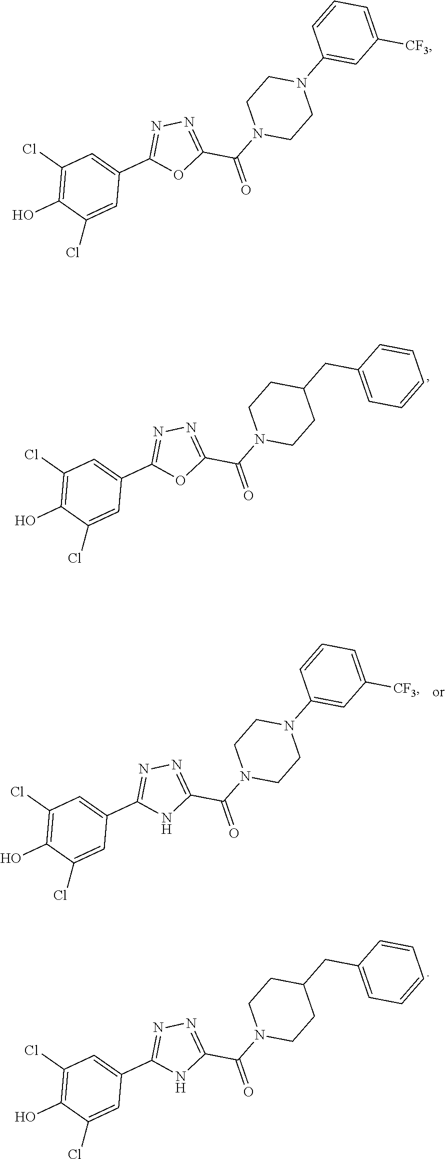 [SMILES: O=C(c1nnc(-c2cc(Cl)c(O)c(Cl)c2)o1)N1CCN(c2cccc(C(F)(F)F)c2)CC1 ; O=C(c1nnc(-c2cc(Cl)c(O)c(Cl)c2)o1)N1CCC(Cc2ccccc2)CC1 ; O=C(c1nnc(-c2cc(Cl)c(O)c(Cl)c2)[nH]1)N1CCN(c2cccc(C(F)(F)F)c2)CC1 ; O=C(c1nnc(-c2cc(Cl)c(O)c(Cl)c2)[nH]1)N1CCC(Cc2ccccc2)CC1]

In some embodiments of a compound of Formula (VIII), the compound is of Formula (VIIIa):

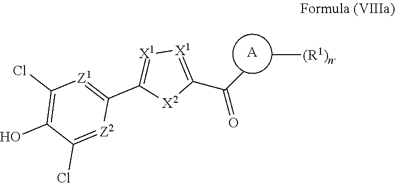

In some embodiments of a compound of Formula (VIII), the compound is of Formula (VIIIb):

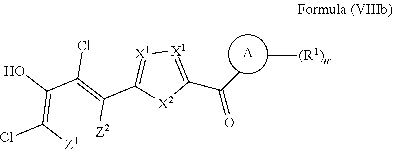

In some embodiments of a compound of Formula (VIII), (VIIIa), or (VIIIb), Ring A is aryl or heteroaryl. In some embodiments of a compound of Formula (VIII), (VIIIa), or (VIIIb), Ring A is phenyl. In some embodiments of a compound of Formula (VIII), (VIIIa), or (VIIIb), Ring A is 5- or 6-membered heteroaryl. In some embodiments of a compound of Formula (VIII), (VIIIa), or (VIIIb), Ring A is pyridinyl, pyrimidinyl, or pyrazinyl. In some embodiments of a compound of Formula (VIII), (VIIIa), or (VIIIb), Ring A is pyridinyl. In some embodiments of a compound of Formula (VIII), (VIIIa), or (VIIIb), Ring A is cycloalkyl or heterocycloalkyl.

In some embodiments of a compound of Formula (VIII), (VIIIa), or (VIIIb), each R¹ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound of Formula (VIII), (VIIIa), or (VIIIb), each R¹ is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound of Formula (VIII), (VIIIa), or (VIIIb), each R¹ is independently deuterium, halogen, —CN, —OH, —OR^{a}, —NR^{c}R^{d}, C₁-C₆alkyl, C₁-C₆haloalkyl, or C₁-C₆deuteroalkyl. In some embodiments of a compound of Formula (VIII), (VIIIa), or (VIIIb), each R¹ is independently deuterium, halogen, —CN, —OH, —OR^{a}, C₁-C₆alkyl, or C₁-C₆haloalkyl.

In some embodiments of a compound of Formula (VIII), (VIIIa), or (VIIIb), n is 0-3. In some embodiments of a compound of Formula (VIII), (VIIIa), or (VIIIb), n is 0-2. In some embodiments of a compound of Formula (VIII), (VIIIa), or (VIIIb), n is 0 or 1. In some embodiments of a compound of Formula (VIII), (VIIIa), or (VIIIb), n is 2. In some embodiments of a compound of Formula (VIII), (VIIIa), or (VIIIb), n is 3.

In some embodiments of a compound of Formula (VIII), (VIIIa), or (VIIIb), Z¹ is N. In some embodiments of a compound of Formula (VIII), (VIIIa), or (VIIIb), Z¹ is CR^{Z1}.

In some embodiments of a compound of Formula (VIII), (VIIIa), or (VIIIb), R^{Z1} is hydrogen, deuterium, halogen, C₁-C₆alkyl, C₁-C₆haloalkyl, or C₁-C₆deuteroalkyl. In some embodiments of a compound of Formula (VIII), (VIIIa), or (VIIIb), R^{Z1} is hydrogen.

In some embodiments of a compound of Formula (VIII), (VIIIa), or (VIIIb), Z² is N. In some embodiments of a compound of Formula (VIII), (VIIIa), or (VIIIb), Z² is CR^{Z2}.

In some embodiments of a compound of Formula (VIII), (VIIIa), or (VIIIb), R^{Z2} is hydrogen, deuterium, halogen, C₁-C₆alkyl, C₁-C₆haloalkyl, or C₁-C₆deuteroalkyl. In some embodiments of a compound of Formula (VIII), (VIIIa), or (VIIIb), R^{Z2} is hydrogen.

In some embodiments of a compound of Formula (VIII), (VIIIa), or (VIIIb), each X¹ is —CR^{X1}—. In some embodiments of a compound of Formula (VIII), (VIIIa), or (VIIIb), each X¹ is —N—. In some embodiments of a compound of Formula (VIII), (VIIIa), or (VIIIb), one X¹ is —N— and the other X¹ is —CR^{X1}—.

In some embodiments of a compound of Formula (VIII), (VIIIa), or (VIIIb), each R^{X1} is hydrogen, deuterium, halogen, or C₁-C₆alkyl optionally substituted with one or more R. In some embodiments of a compound of Formula (VIII), (VIIIa), or (VIIIb), each R^{X1} is hydrogen or C₁-C₆alkyl. In some embodiments of a compound of Formula (VIII), (VIIIa), or (VIIIb), each R^{X1} is hydrogen.

In some embodiments of a compound of Formula (VIII), (VIIIa), or (VIIIb), X² is —O—. In some embodiments of a compound of Formula (VIII), (VIIIa), or (VIIIb), X² is —S—. In some embodiments of a compound of Formula (VIII), (VIIIa), or (VIIIb), X² is —NR^{X2}—.

In some embodiments of a compound of Formula (VIII), (VIIIa), or (VIIIb), R^{X2} is hydrogen or C₁-C₆alkyl.

In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, cycloalkyl, heterocycloalkyl, aryl, and heteroaryl is independently optionally substituted with one or more R. In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl); wherein each alkyl, alkylene, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound disclosed herein, each R^{a} is independently C₁-C₆alkyl, C₁-C₆haloalkyl, C₁-C₆deuteroalkyl, cycloalkyl, or heterocycloalkyl; wherein each alkyl, cycloalkyl, and heterocycloalkyl is independently optionally substituted with one or more R. In some embodiments of a compound disclosed herein, each R^{a} is independently C₁-C₆alkyl, C₁-C₆haloalkyl, or C₁-C₆deuteroalkyl; wherein each alkyl is independently optionally substituted with one or more R. In some embodiments of a compound disclosed herein, each R^{a} is independently C₁-C₆alkyl, C₁-C₆haloalkyl, or C₁-C₆deuteroalkyl. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl.

In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(ayl), or C₁-C₆alkylene(heteroaryl); wherein each alkyl, alkylene, cycloalkyl, heterocycloalkyl, aryl, and heteroaryl is independently optionally substituted with one or more R. In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl); wherein each alkyl, alkylene, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆deuteroalkyl, cycloalkyl, or heterocycloalkyl; wherein each alkyl, cycloalkyl, and heterocycloalkyl is independently optionally substituted with one or more R. In some embodiments of a compound disclosed herein, each R^{b} is independently hydrogen, C₁-C₆alkyl, C₁-C₆haloalkyl, or C₁-C₆deuteroalkyl; wherein each alkyl is independently optionally substituted with one or more R. In some embodiments of a compound disclosed herein, each R^{b} is independently hydrogen, C₁-C₆alkyl, C₁-C₆haloalkyl, or C₁-C₆deuteroalkyl. In some embodiments of a compound disclosed herein, each R^{b} is independently hydrogen, C₁-C₆alkyl, or C₁-C₆haloalkyl. In some embodiments of a compound disclosed herein, each R^{b} is independently hydrogen or C₁-C₆alkyl. In some embodiments of a compound disclosed herein, each R^{b} is independently hydrogen. In some embodiments of a compound disclosed herein, each R^{b} is independently C₁-C₆alkyl.

In some embodiments of a compound disclosed herein, each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, cycloalkyl, heterocycloalkyl, aryl, and heteroaryl is independently optionally substituted with one or more R. In some embodiments of a compound disclosed herein, each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, C₁-C₆alkylene(cycloalkyl), or C₁-C₆alkylene(heterocycloalkyl); wherein each alkyl, alkylene, cycloalkyl, and heterocycloalkyl is independently optionally substituted with one or more R. In some embodiments of a compound disclosed herein, each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments of a compound disclosed herein, each R^{c} and R^{d} are independently hydrogen, C₁-C₆alkyl, C₁-C₆haloalkyl, C₁-C₆deuteroalkyl, cycloalkyl, or heterocycloalkyl; wherein each alkyl, cycloalkyl, and heterocycloalkyl is independently optionally substituted with one or more R. In some embodiments of a compound disclosed herein, each R^{c} and R^{d} are independently hydrogen, C₁-C₆alkyl, C₁-C₆haloalkyl, or C₁-C₆deuteroalkyl; wherein each alkyl is independently optionally substituted with one or more R. In some embodiments of a compound disclosed herein, each R^{c} and R^{d} are independently hydrogen, C₁-C₆alkyl, C₁-C₆haloalkyl, or C₁-C₆deuteroalkyl. In some embodiments of a compound disclosed herein, each R^{c} and R^{d} are independently hydrogen, C₁-C₆alkyl, or C₁-C₆haloalkyl. In some embodiments of a compound disclosed herein, each R^{c} and R^{d} are independently hydrogen or C₁-C₆alkyl. In some embodiments of a compound disclosed herein, each R^{c} and R^{d} are hydrogen. In some embodiments of a compound disclosed herein, each R^{c} and R^{d} are independently C₁-C₆alkyl.

In some embodiments of a compound disclosed herein, R^{c} and R^{d} are taken together with the atom to which they are attached to form a heterocycloalkyl optionally substituted with one or more R.

In some embodiments of a compound disclosed herein, each R is independently halogen, —CN, —OH, —OC₁-C₃alkyl, —OC₁-C₃haloalkyl, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl; or two R on the same atom are taken together to form an oxo. In some embodiments of a compound disclosed herein, each R is independently halogen, —CN, —OH, —OC₁-C₃alkyl, —OC₁-C₃haloalkyl, —NH₂, —NHC₁-C₃alkyl, —N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl; or two R on the same atom are taken together to form an oxo. In some embodiments of a compound disclosed herein, each R is independently halogen, —CN, —OH, —OC₁-C₃alkyl, —OC₁-C₃haloalkyl, —NH₂, C₁-C₃alkyl, C₁-C₃haloalkyl, C₁-C₃deuteroalkyl; or two R on the same atom are taken together to form an oxo. In some embodiments of a compound disclosed herein, each R is independently halogen, —CN, —OH, —NH₂, C₁-C₃alkyl, or C₁-C₃haloalkyl; or two R on the same atom are taken together to form an oxo. In some embodiments of a compound disclosed herein, each R is independently halogen, —CN, —OH, —NH₂, C₁-C₃alkyl, or C₁-C₃haloalkyl. In some embodiments of a compound disclosed herein, each R is independently halogen, C₁-C₃alkyl, or C₁-C₃haloalkyl. In some embodiments of a compound disclosed herein, each R is independently halogen or C₁-C₃alkyl.

Any combination of the groups described above for the various variables is contemplated herein. Throughout the specification, groups and substituents thereof are chosen by one skilled in the field to provide stable moieties and compounds.

Described herein is a compound, or a pharmaceutically acceptable salt, solvate, or stereoisomer thereof, selected from:

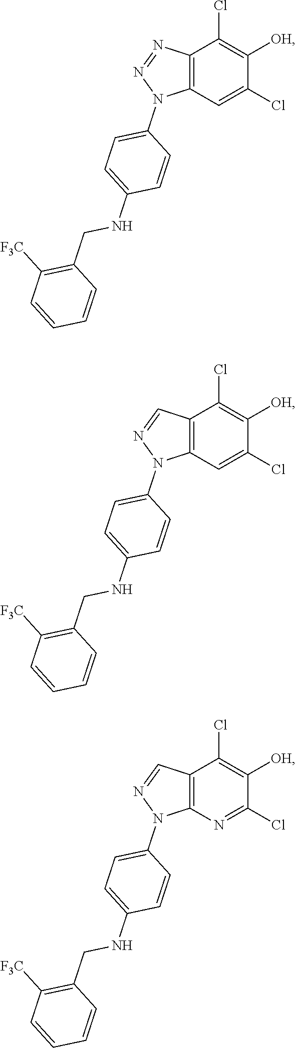 [SMILES: Oc1c(Cl)cc2c(nnn2-c2ccc(NCc3ccccc3C(F)(F)F)cc2)c1Cl ; Oc1c(Cl)cc2c(cnn2-c2ccc(NCc3ccccc3C(F)(F)F)cc2)c1Cl ; Oc1c(Cl)nc2c(cnn2-c2ccc(NCc3ccccc3C(F)(F)F)cc2)c1Cl]

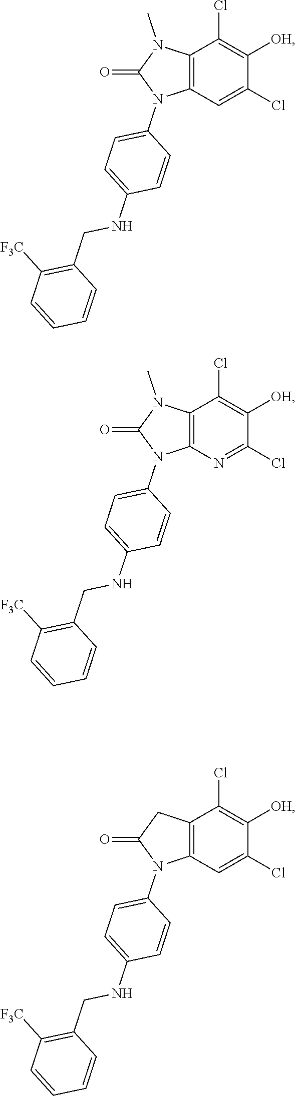 [SMILES: Cn1c(=O)n(-c2ccc(NCc3ccccc3C(F)(F)F)cc2)c2cc(Cl)c(O)c(Cl)c21 ; Cn1c(=O)n(-c2ccc(NCc3ccccc3C(F)(F)F)cc2)c2nc(Cl)c(O)c(Cl)c21 ; O=C1Cc2c(cc(Cl)c(O)c2Cl)N1c1ccc(NCc2ccccc2C(F)(F)F)cc1]

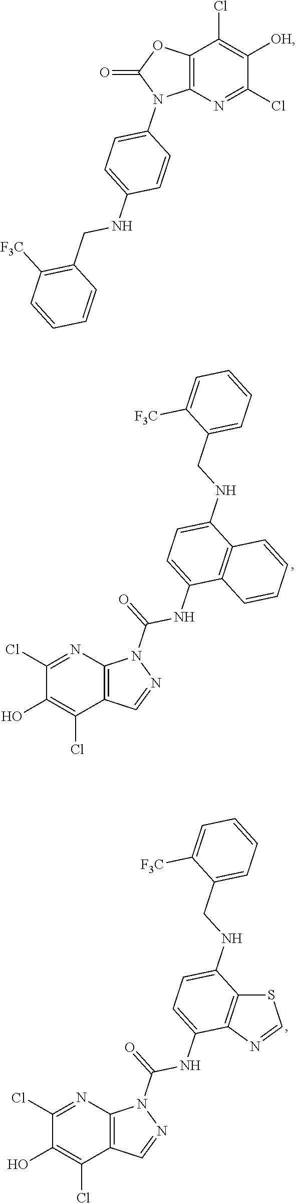 [SMILES: O=c1oc2c(Cl)c(O)c(Cl)nc2n1-c1ccc(NCc2ccccc2C(F)(F)F)cc1 ; O=C(Nc1ccc(NCc2ccccc2C(F)(F)F)c2ccccc12)n1ncc2c(Cl)c(O)c(Cl)nc21 ; O=C(Nc1ccc(NCc2ccccc2C(F)(F)F)c2scnc12)n1ncc2c(Cl)c(O)c(Cl)nc21]

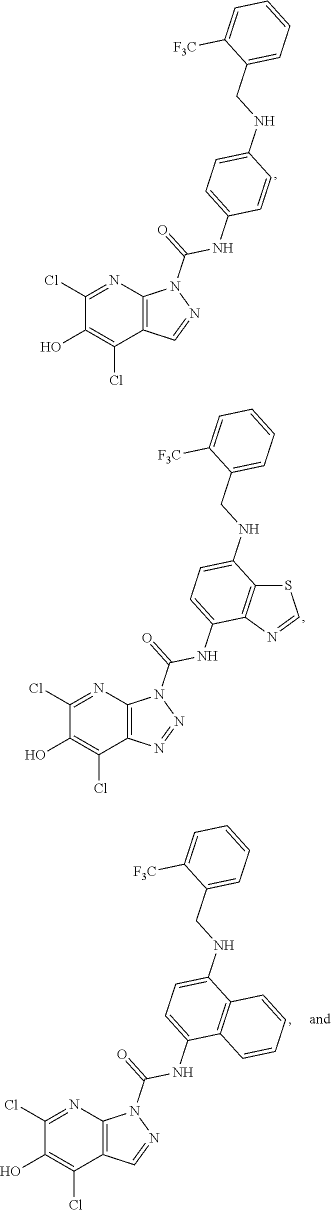 [SMILES: O=C(Nc1ccc(NCc2ccccc2C(F)(F)F)cc1)n1ncc2c(Cl)c(O)c(Cl)nc21 ; O=C(Nc1ccc(NCc2ccccc2C(F)(F)F)c2scnc12)n1nnc2c(Cl)c(O)c(Cl)nc21 ; O=C(Nc1ccc(NCc2ccccc2C(F)(F)F)c2ccccc12)n1ncc2c(Cl)c(O)c(Cl)nc21]

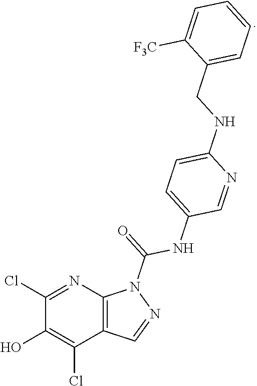 [SMILES: O=C(Nc1ccc(NCc2ccccc2C(F)(F)F)nc1)n1ncc2c(Cl)c(O)c(Cl)nc21]

Described herein is a compound, or a pharmaceutically acceptable salt, solvate, or stereoisomer thereof, selected from:

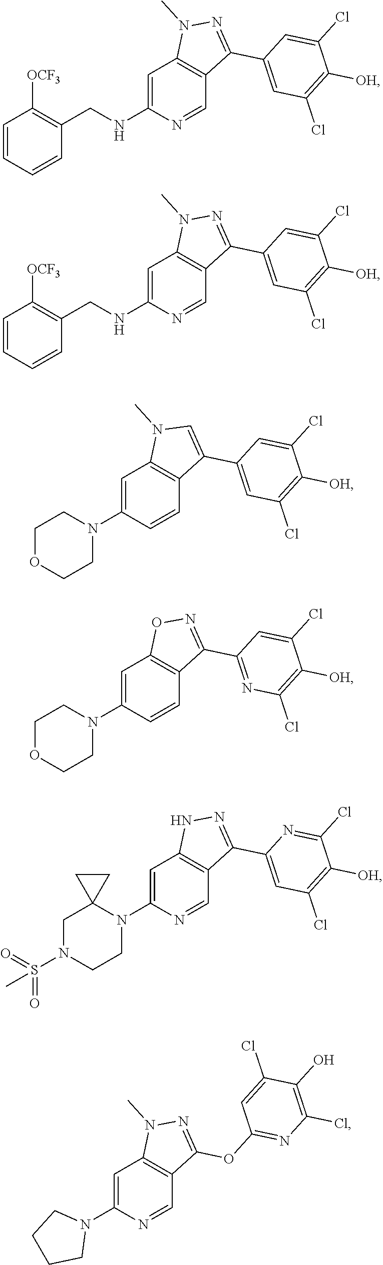 [SMILES: Cn1nc(-c2cc(Cl)c(O)c(Cl)c2)c2cnc(NCc3ccccc3OC(F)(F)F)cc21 ; Cn1nc(-c2cc(Cl)c(O)c(Cl)c2)c2cnc(NCc3ccccc3OC(F)(F)F)cc21 ; Cn1cc(-c2cc(Cl)c(O)c(Cl)c2)c2ccc(N3CCOCC3)cc21 ; Oc1c(Cl)cc(-c2noc3cc(N4CCOCC4)ccc23)nc1Cl ; CS(=O)(=O)N1CCN(c2cc3[nH]nc(-c4cc(Cl)c(O)c(Cl)n4)c3cn2)C2(CC2)C1 ; Cn1nc(Oc2cc(Cl)c(O)c(Cl)n2)c2cnc(N3CCCC3)cc21]

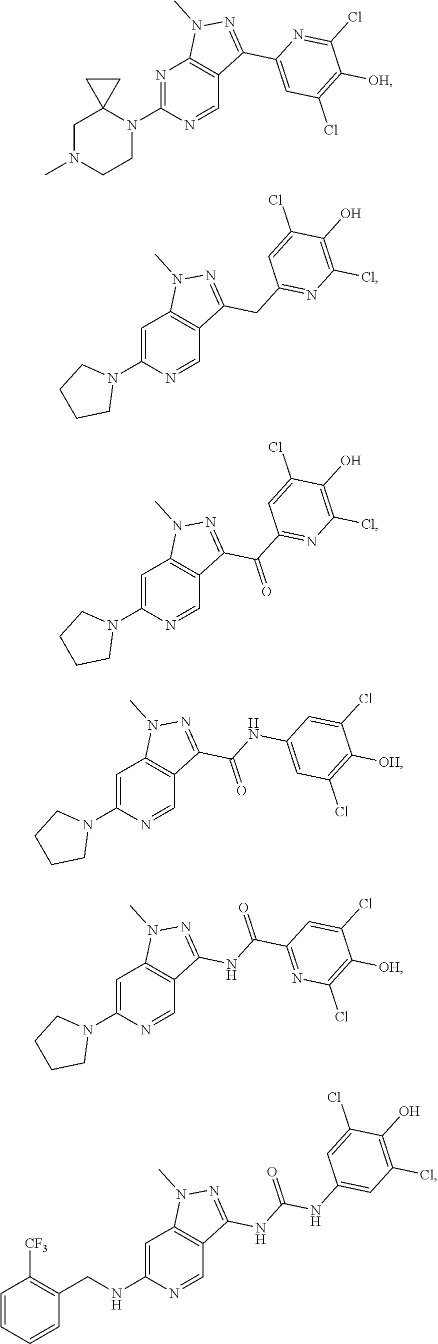 [SMILES: CN1CCN(c2ncc3c(-c4cc(Cl)c(O)c(Cl)n4)nn(C)c3n2)C2(CC2)C1 ; Cn1nc(Cc2cc(Cl)c(O)c(Cl)n2)c2cnc(N3CCCC3)cc21 ; Cn1nc(C(=O)c2cc(Cl)c(O)c(Cl)n2)c2cnc(N3CCCC3)cc21 ; Cn1nc(C(=O)Nc2cc(Cl)c(O)c(Cl)c2)c2cnc(N3CCCC3)cc21 ; Cn1nc(NC(=O)c2cc(Cl)c(O)c(Cl)n2)c2cnc(N3CCCC3)cc21 ; Cn1nc(NC(=O)Nc2cc(Cl)c(O)c(Cl)c2)c2cnc(NCc3ccccc3C(F)(F)F)cc21]

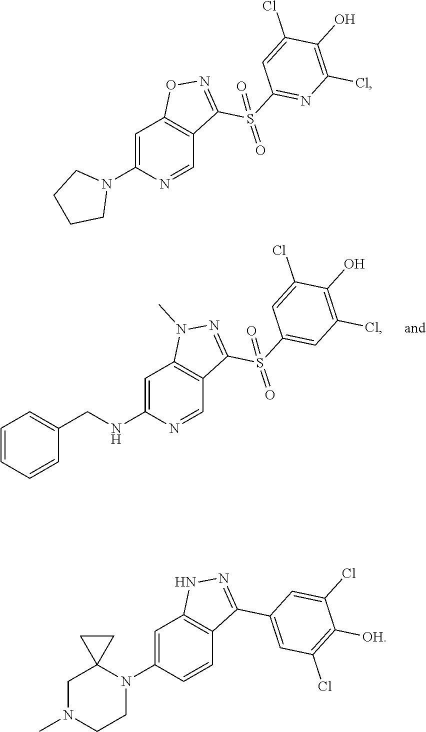 [SMILES: O=S(=O)(c1cc(Cl)c(O)c(Cl)n1)c1noc2cc(N3CCCC3)ncc12 ; Cn1nc(S(=O)(=O)c2cc(Cl)c(O)c(Cl)c2)c2cnc(NCc3ccccc3)cc21 ; CN1CCN(c2ccc3c(-c4cc(Cl)c(O)c(Cl)c4)n[nH]c3c2)C2(CC2)C1]

Described herein is a compound, or a pharmaceutically acceptable salt, solvate, or stereoisomer thereof, selected from:

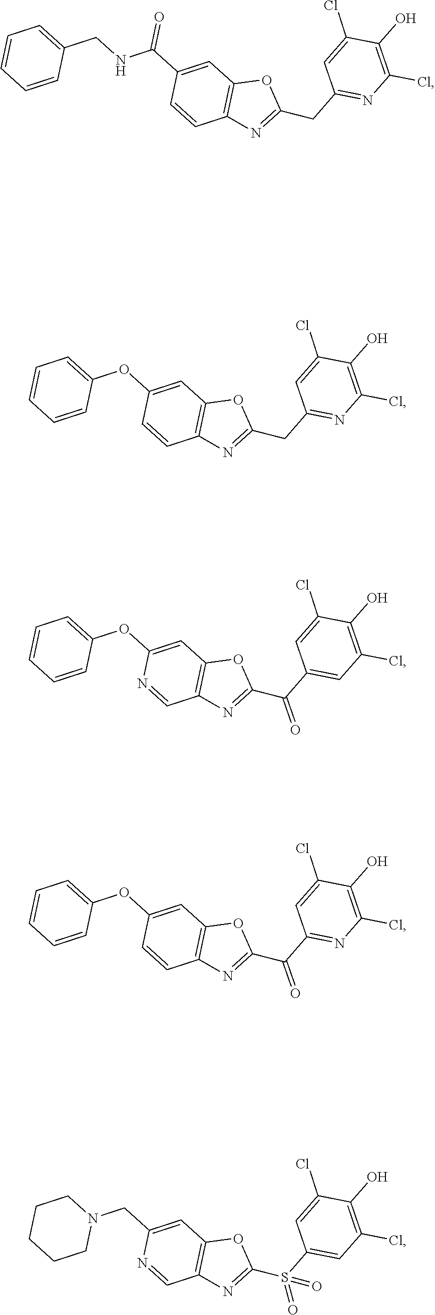 [SMILES: O=C(NCc1ccccc1)c1ccc2nc(Cc3cc(Cl)c(O)c(Cl)n3)oc2c1 ; Oc1c(Cl)cc(Cc2nc3ccc(Oc4ccccc4)cc3o2)nc1Cl ; O=C(c1cc(Cl)c(O)c(Cl)c1)c1nc2cnc(Oc3ccccc3)cc2o1 ; O=C(c1cc(Cl)c(O)c(Cl)n1)c1nc2ccc(Oc3ccccc3)cc2o1 ; O=S(=O)(c1cc(Cl)c(O)c(Cl)c1)c1nc2cnc(CN3CCCCC3)cc2o1]

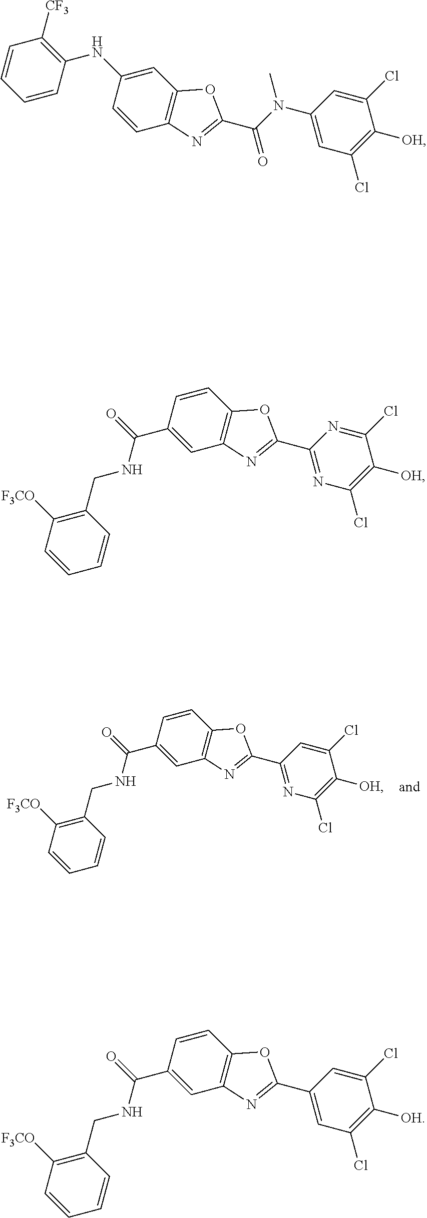 [SMILES: CN(C(=O)c1nc2ccc(Nc3ccccc3C(F)(F)F)cc2o1)c1cc(Cl)c(O)c(Cl)c1 ; O=C(NCc1ccccc1OC(F)(F)F)c1ccc2oc(-c3nc(Cl)c(O)c(Cl)n3)nc2c1 ; O=C(NCc1ccccc1OC(F)(F)F)c1ccc2oc(-c3cc(Cl)c(O)c(Cl)n3)nc2c1 ; O=C(NCc1ccccc1OC(F)(F)F)c1ccc2oc(-c3cc(Cl)c(O)c(Cl)c3)nc2c1]

Described herein is a compound, or a pharmaceutically acceptable salt, solvate, or stereoisomer thereof, selected from:

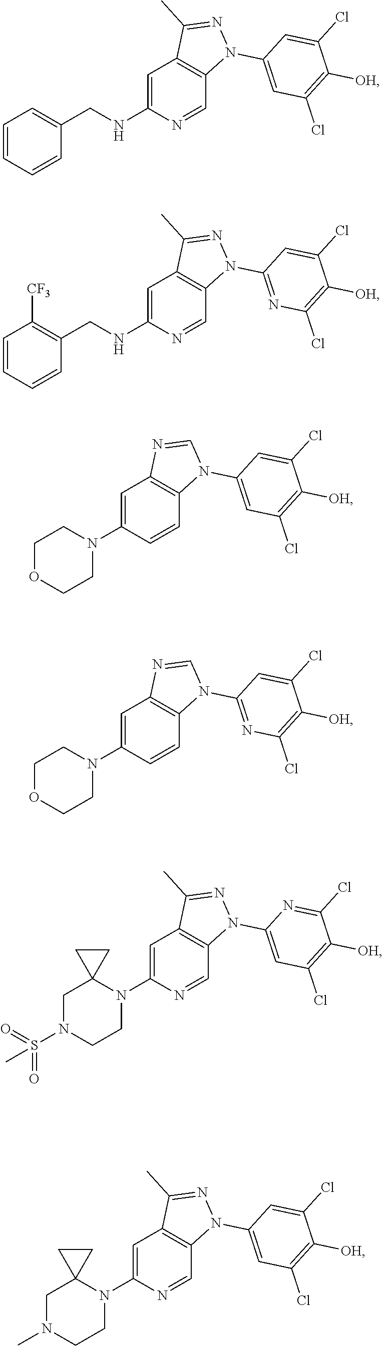 [SMILES: Cc1nn(-c2cc(Cl)c(O)c(Cl)c2)c2cnc(NCc3ccccc3)cc12 ; Cc1nn(-c2cc(Cl)c(O)c(Cl)n2)c2cnc(NCc3ccccc3C(F)(F)F)cc12 ; Oc1c(Cl)cc(-n2cnc3cc(N4CCOCC4)ccc32)cc1Cl ; Oc1c(Cl)cc(-n2cnc3cc(N4CCOCC4)ccc32)nc1Cl ; Cc1nn(-c2cc(Cl)c(O)c(Cl)n2)c2cnc(N3CCN(S(C)(=O)=O)CC34CC4)cc12 ; Cc1nn(-c2cc(Cl)c(O)c(Cl)c2)c2cnc(N3CCN(C)CC34CC4)cc12]

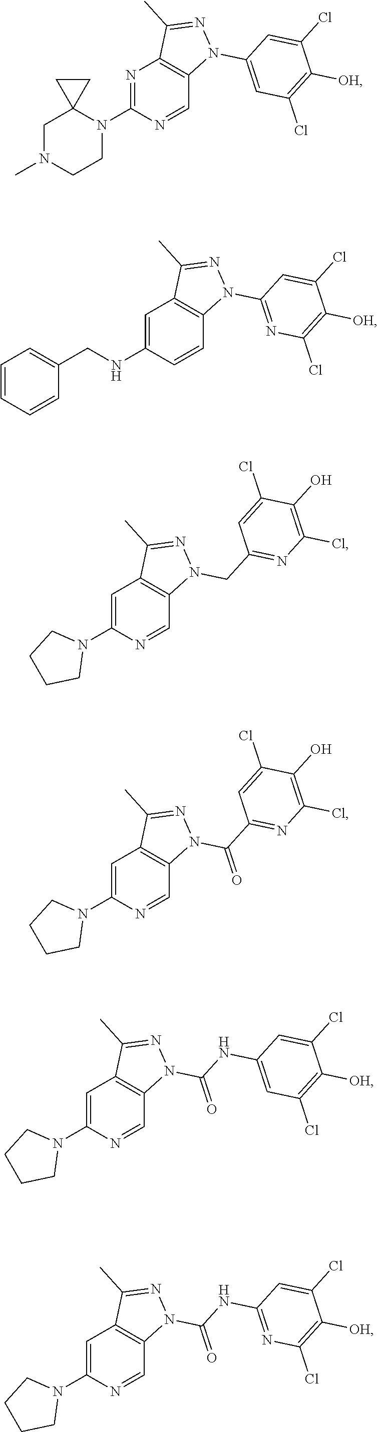 [SMILES: Cc1nn(-c2cc(Cl)c(O)c(Cl)c2)c2cnc(N3CCN(C)CC34CC4)nc12 ; Cc1nn(-c2cc(Cl)c(O)c(Cl)n2)c2ccc(NCc3ccccc3)cc12 ; Cc1nn(Cc2cc(Cl)c(O)c(Cl)n2)c2cnc(N3CCCC3)cc12 ; Cc1nn(C(=O)c2cc(Cl)c(O)c(Cl)n2)c2cnc(N3CCCC3)cc12 ; Cc1nn(C(=O)Nc2cc(Cl)c(O)c(Cl)c2)c2cnc(N3CCCC3)cc12 ; Cc1nn(C(=O)Nc2cc(Cl)c(O)c(Cl)n2)c2cnc(N3CCCC3)cc12]

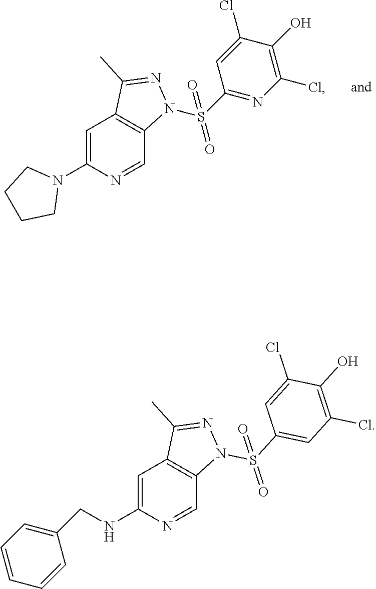 [SMILES: Cc1nn(S(=O)(=O)c2cc(Cl)c(O)c(Cl)n2)c2cnc(N3CCCC3)cc12 ; Cc1nn(S(=O)(=O)c2cc(Cl)c(O)c(Cl)c2)c2cnc(NCc3ccccc3)cc12]

Described herein is a compound, or a pharmaceutically acceptable salt, solvate, or stereoisomer thereof, selected from:

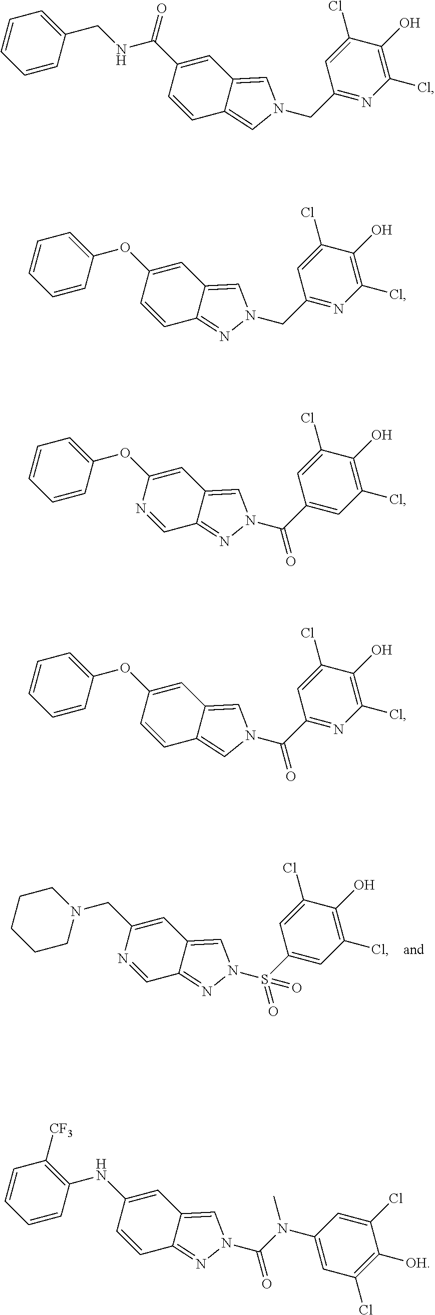 [SMILES: O=C(NCc1ccccc1)c1ccc2cn(Cc3cc(Cl)c(O)c(Cl)n3)cc2c1 ; Oc1c(Cl)cc(Cn2cc3cc(Oc4ccccc4)ccc3n2)nc1Cl ; O=C(c1cc(Cl)c(O)c(Cl)c1)n1cc2cc(Oc3ccccc3)ncc2n1 ; O=C(c1cc(Cl)c(O)c(Cl)n1)n1cc2ccc(Oc3ccccc3)cc2c1 ; O=S(=O)(c1cc(Cl)c(O)c(Cl)c1)n1cc2cc(CN3CCCCC3)ncc2n1 ; CN(C(=O)n1cc2cc(Nc3ccccc3C(F)(F)F)ccc2n1)c1cc(Cl)c(O)c(Cl)c1]

Described herein is a compound, or a pharmaceutically acceptable salt, solvate, or stereoisomer thereof, selected from:

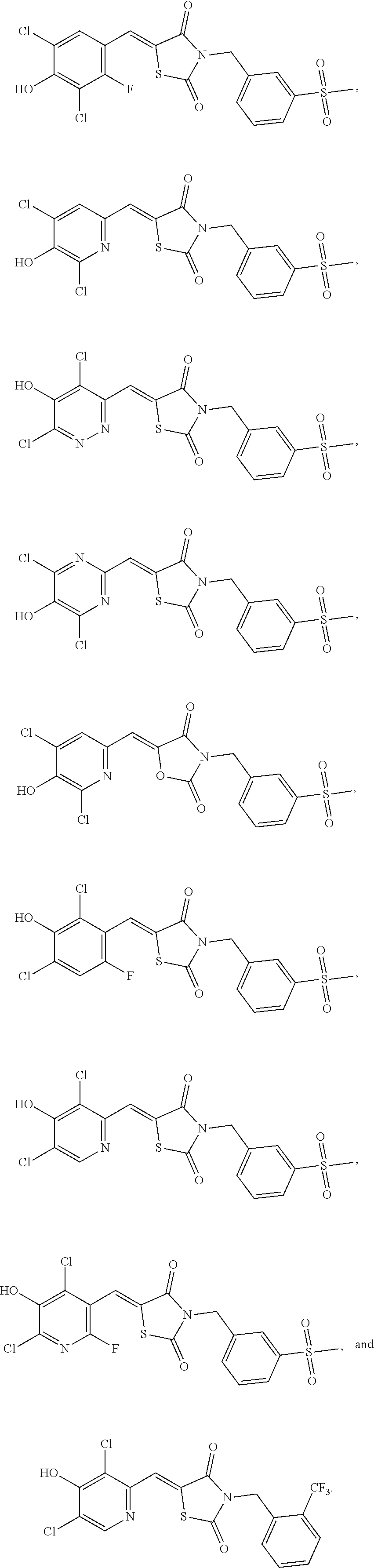

Described herein is a compound, or a pharmaceutically acceptable salt, solvate, or stereoisomer thereof, selected from:

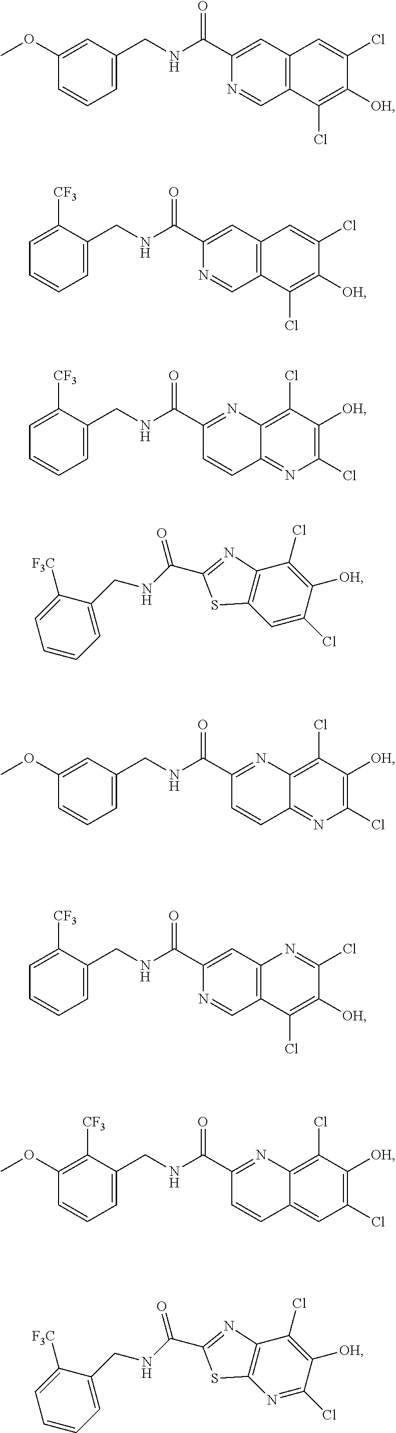

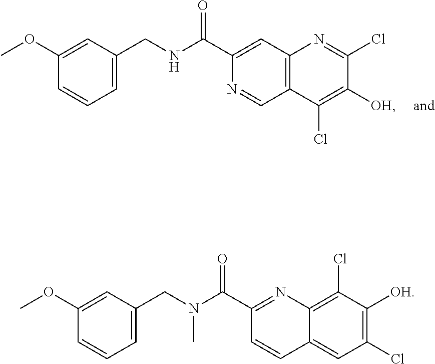 [SMILES: COc1cccc(CNC(=O)c2cc3nc(Cl)c(O)c(Cl)c3cn2)c1 ; COc1cccc(CN(C)C(=O)c2ccc3cc(Cl)c(O)c(Cl)c3n2)c1]

Described herein is a compound, or a pharmaceutically acceptable salt, solvate, or stereoisomer thereof, selected from:

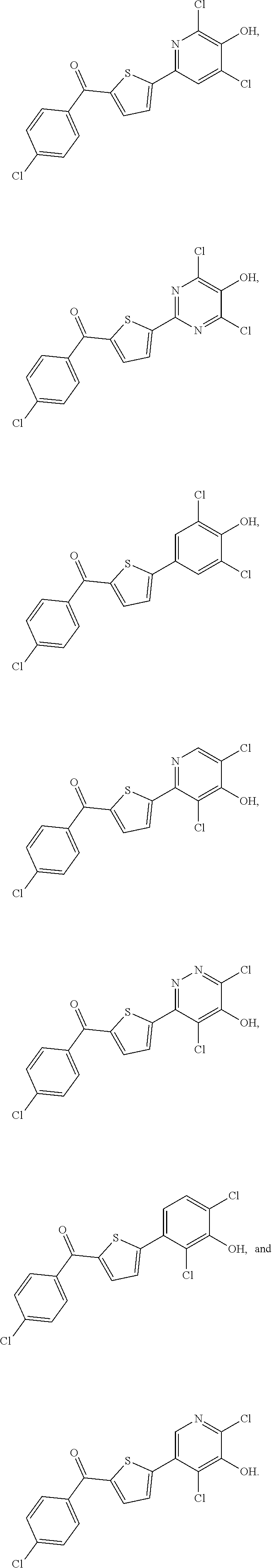

Described herein is a compound, or a pharmaceutically acceptable salt, solvate, or stereoisomer thereof, selected from:

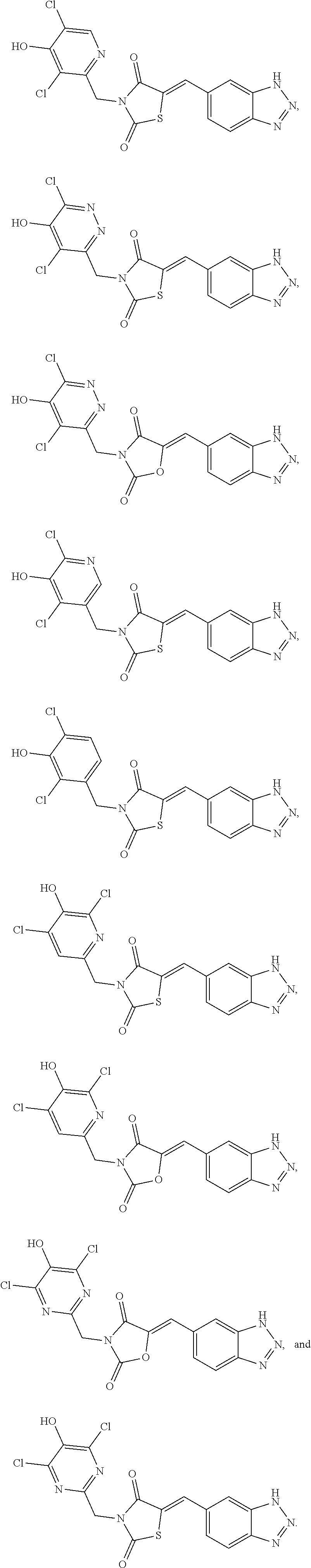

### Further Forms of Compounds Disclosed Herein

### Isomers Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements.

In some embodiments, the compounds described herein are labeled by other means, including, but not limited to, the use of chromophores or fluorescent moieties, bioluminescent labels, or chemiluminescent labels.

In some embodiments, the labeled compounds described herein are used for measuring in vitro and in vivo binding of unlabeled HSD17B13 inhibitors.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Provided herein are methods of inhibiting HSD17B13 expression or activity, which can be useful for treating, preventing, or ameliorating a disease associated with HSD17B13 in a subject in need thereof, such as NAFLD or NASH, by administration of a compound that targets HSD17B13, or a pharmaceutically acceptable salt, solvate, or stereoisomer thereof.

Provided herein are methods of inhibiting expression or activity of HSD17B13 in a cell comprising contacting the cell with a HSD17B13 inhibitor disclosed or a pharmaceutically acceptable salt, solvate, or stereoisomer thereof, thereby inhibiting expression or activity of HSD17B13 in the cell. In some embodiments, the cell is a hepatocyte cell. In some embodiments, the cell is in the liver. In some embodiments, the cell is in the liver of a subject who has, or is at risk of having a disease, disorder, condition, symptom, or physiological marker associated with a liver disease, metabolic disease, or cardiovascular disease or disorder. In some embodiments, the cells are the adipocytes or monocytes from a subject who has or is at risk of having a disease. In some embodiments, the cells are the lymphocytes from a subject who has or is at risk of having a disease. In some embodiments, the liver disease, metabolic disease, or cardiovascular disease or disorder is metabolic syndrome, fatty liver disease, chronic liver disease, liver cirrhosis, hepatic steatosis, steatohepatitis, nonalcoholic fatty liver disease (NAFLD), alcoholic liver disease, nonalcoholic steatohepatitis (NASH), fulminant Wilson's disease, rapidly fibrosing hepatitis C viral injury, and decompensated portal vein hypertension. In some embodiments, the liver disease, metabolic disease, or cardiovascular disease or disorder is NASH. In some embodiments, the liver disease, metabolic disease, or cardiovascular disease or disorder is cholestatic liver disease.

In some embodiments, the liver disease is primary biliary cirrhosis or primary sclerosing cholangitis.

Provided herein are methods of treating, preventing, delaying the onset, slowing the progression, or ameliorating one or more diseases, disorders, conditions, symptoms, or physiological markers associated with HSD17B13 comprising administering to a subject in need thereof a compound disclosed herein, or a pharmaceutically acceptable salt, solvate, or stereoisomer thereof. In some embodiments, the subject in need thereof is identified as having, or at risk of having, the disease, disorder, condition, symptom, or physiological marker. In some embodiments, the liver disease, metabolic disease, or cardiovascular disease or disorder is metabolic syndrome, liver disease, fatty liver disease, chronic liver disease, liver cirrhosis, hepatic steatosis, steatohepatitis, alcoholic liver disease, nonalcoholic fatty liver disease (NAFLD), and nonalcoholic steatohepatitis (NASH). In some embodiments, the liver disease, metabolic disease, or cardiovascular disease or disorder is NASH.

Provided herein are methods of reducing, improving, or regulating hepatic steatosis, liver fibrosis, triglyceride synthesis, lipid levels, hepatic lipids, ALT levels, NAFLD Activity Score (NAS), cholesterol levels, or triglyceride levels, or a combination thereof, in a subject in need thereof comprising administering to the subject a compound disclosed herein, or a pharmaceutically acceptable salt, solvate, or stereoisomer thereof. In some embodiments, the compound disclosed herein, or a pharmaceutically acceptable salt, solvate, or stereoisomer thereof, is provided for use in reducing, improving, or regulating hepatic steatosis in the individual. In some embodiments, the compound disclosed herein, or a pharmaceutically acceptable salt, solvate, or stereoisomer thereof, is provided for use in reducing, improving, or regulating liver fibrosis in the individual. In some embodiments, the compound disclosed herein, or a pharmaceutically acceptable salt, solvate, or stereoisomer thereof, is provided for use in reducing, improving, or regulating triglyceride synthesis in the individual. In some embodiments, the compound disclosed herein, or a pharmaceutically acceptable salt, solvate, or stereoisomer thereof, is provided for use in reducing, improving, or regulating lipid levels in the individual. In some embodiments, the compound disclosed herein, or a pharmaceutically acceptable salt, solvate, or stereoisomer thereof, is provided for use in reducing, improving, or regulating hepatic lipids in the individual. In some embodiments, the compound disclosed herein, or a pharmaceutically acceptable salt, solvate, or stereoisomer thereof, is provided for use in reducing, improving, or regulating ALT levels in the individual. In some embodiments, the compound disclosed herein, or a pharmaceutically acceptable salt, solvate, or stereoisomer thereof, is provided for use in reducing, improving, or regulating NAFLD Activity Score in the individual. In some embodiments, the compound disclosed herein, or a pharmaceutically acceptable salt, solvate, or stereoisomer thereof, is provided for use in reducing, improving, or regulating cholesterol levels in the individual. In some embodiments, the compound disclosed herein, or a pharmaceutically acceptable salt, solvate, or stereoisomer thereof, is provided for use in reducing, improving, or regulating triglyceride levels in the individual. In some embodiments, the subject is identified as having, or at risk of having a disease, disorder, condition, symptom, or physiological marker associated with a liver disease, metabolic disease, or cardiovascular disease or disorder. In some embodiments, the liver disease, metabolic disease, or cardiovascular disease or disorder is metabolic syndrome, liver disease, fatty liver disease, chronic liver disease, liver cirrhosis, hepatic steatosis, steatohepatitis, nonalcoholic fatty liver disease (NAFLD), and nonalcoholic steatohepatitis (NASH). In some embodiments, the liver disease, metabolic disease, or cardiovascular disease or disorder is NASH.

Provided herein are methods for treating, preventing, or delaying onset drug induced liver injury (DILI) in a subject in need thereof, comprising administering to the subject a compound disclosed herein, or a pharmaceutically acceptable salt, solvate, or stereoisomer thereof. In some embodiments, the liver injury is steatohepatitis. Also provided herein are methods for treating, preventing, or delaying onset drug induced steatohepatitis (DISH) in a subject in need thereof. In some embodiments, the subject in need thereof is receiving chemotherapy for treating cancer. In some embodiments, the subject in need thereof is receiving a treatment for a cardiovascular disease. In some embodiments, the subject in need thereof is receiving treatment for a psychiatric disease/condition. In some embodiments, the subject in need thereof is receiving treatment for pain. In some embodiments, the subject in need thereof is receiving treatment for arthritis. In some embodiments, the chemotherapy is tamoxifen, toremifene, irinotecan, methotrexate, fluorouracil (5-FU), or any combination thereof. In some embodiments, the subject in need thereof is receiving amiodarone, perhexiline, propranolol, or any combination thereof. In some embodiments, the subject in need thereof is receiving amitriptyline, clozapine, or any combination thereof. In some embodiments, the subject in need thereof is receiving methotrexate, pirprofen, or any combinations thereof.

### Cholestatic Diseases

Provided herein is a method of treating a cholestatic disease in a subject in need thereof, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof.

In some embodiments, inhibiting HSD17B13 improves bile flow. In some embodiments, inhibition of HSD17B13 is used to treat a cholestatic disease. Cholestatic diseases include primary sclerosis cholangitis (PSC), primary biliary cholangitis (PBC), Alagille Syndrome, biliary atresia, liver injury in cystic fibrosis patients and progressive familial intrahepatic cholestasis (PFIC).

HSD17B13 protein is very highly expressed in liver and gallbladder. The gallbladder is developmentally downstream of hepatocytes through the biliary tree. Inactive HSD17B13 has been associated with increases in liver and plasma phosphatidylcholine. Phosphatidylcholine is essential for bile flow. Hepatic phosphatidylcholine is secreted into the bile at a rate equivalent to the total liver levels of phosphatidylcholine being secreted within a day along with bile acids (BAs) and cholesterol. In some embodiments, inactive HSD17B13 is associated with increased plasma levels of VLDL-cholesterol. Meaning that there is a greater secretion of cholesterol out of the liver and not catabolized to bile acids for secretion in bile. In some embodiments, inhibition of HSD17B13 improves bile flow through increased phosphatidylcholine. In some embodiments, HSD17B13 is protecting the biliary tree by preventing inflammation. In some embodiments, HSD17B13 is protecting the biliary tree by preventing the cytotoxic bile acids from injuring the biliary tree.

In some embodiments, the cholestatic disease is primary sclerosis cholangitis (PSC), primacy biliary cholangitis (PBC), Alagille Syndrome, biliary atresia, liver injury in a cystic fibrosis patient, progressive familial intrahepatic cholestasis (PFIC), intrahepatic cholestasis of pregnancy; drug-induced cholestasis, AIDS cholangiopathy, IG4-associated cholangitis, biliary stricture, or low phospholipid-associated cholestasis.

In some embodiments, the cholestatic disease is primary sclerosis cholangitis (PSC), primary biliary cholangitis (PBC), Alagille Syndrome, biliary atresia, liver injury in a cystic fibrosis patient, or progressive familial intrahepatic cholestasis (PFIC).

In some embodiments, the cholestatic disease is primary sclerosis cholangitis (PSC).

In some embodiments, the PSC is accompanied by inflammatory bowel disease (IBD). In some embodiments, the PSC is accompanied by elevated levels of lipopolysaccharide (LPS) (endotoxemia). In some embodiments, the elevated levels of LPS is in the blood. In some embodiments, the elevated levels of LPS is in the liver. In some embodiments, the elevated levels of LPS is in the biliary tree. In some embodiments, the cholestatic disease is primary biliary cholangitis (PBC). In some embodiments, the cholestatic disease is Alagille Syndrome. In some embodiments, the cholestatic disease is biliary atresia. In some embodiments, the cholestatic disease is liver injury in a cystic fibrosis patient. In some embodiments, the cholestatic disease is progressive familial intrahepatic cholestasis (PFIC). In some embodiments, the PFIC is PFIC-3 type. In some embodiments, the PFIC-3 type is due to a mutation in ABCB4 which requires phosphatidylcholine for bile acid transport.

In some embodiments, the cholestatic disease is treated by improving bile flow in the subject in need thereof. In some embodiments, the cholestatic disease is treated by improving cholesterol secretion out of the liver in the subject in need thereof.

In some embodiments, inactive HSD17B13 is associated to lower cytokines and inflammatory gene expression. In some embodiments, there is an improvement in the hepatocyte response to LPS in hepatocytes with inactive HSD17B13. In some embodiments, inactive HSD17B13 is associated with improved autophagy in response to LPS (see FIG. 1A, FIG. 1B, and FIG. 1C). In some embodiment, increases in LC3B-11 combined with increases in p62 indicate accumulation of autophagosomes and defective autophagy.

### Liver Injury Due to Protein Accumulation

Disclosed herein is a method of treating liver injury due to protein accumulation in a subject in need thereof, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof. In some embodiments, the protein that accumulates is alpha 1-antitrypsin.

Alpha 1-antitrypsin is encoded by the gene SERPINA1. The normal allele is referred to as M, while common SERPINA1 alleles that have been associated with liver disease have amino acid changes Glu264Val, referred to as S and Glu342Lys, referred to as Z. Individuals that are homozygotes for the Z (designated PiZZ) allele, have pulmonary manifestations of their A1AT deficiency and can be treated by supplementation of the enzyme. However, individuals that are heterozygotes for the Z allele either with the M (designated PiMZ) or with the S allele (designated PiSZ) have liver disease manifestations. The protein resulting from translation of the Z and S alleles is a misfolded protein. This misfolded protein has been found to polymerize and lead to cellular injury due to accumulation of misfolded and polymerized protein. Protection against the injury due to accumulated misfolded protein has been observed by increasing autophagy.

In some embodiments, HSD17B13 plays a role in autophagy. In some embodiments, autophagy is important for eliminating misfolded proteins and has been implicated in liver injury due to alpha 1-antitrypsin deficiency. In some embodiments, inhibition of HSD17B13 improves autophagy and thus improve clearance of misfolded proteins and thus, improve liver health. In some embodiments, liver disease associated with alpha 1-antitrypsin deficiency include inflammation and decreases in platelets. In some embodiments, inactive HSD17B13 is associated with lower inflammation, decreased inflammatory genes including NF-kB and TGF-β as well as increases in platelets.

In some embodiments, the protein accumulation is cleared via autophagy. In some embodiments, the protein is a misfolded protein. In some embodiments, the liver injury is due to alpha 1-antitrypsin deficiency. In some embodiments, the alpha 1-antitrypsin deficiency results in a protein that does not get fully processed and accumulates as a mis-folded protein in the liver. In some embodiments, the liver disease associated with alpha 1-antitrypsin deficiency include inflammation. In some embodiments, the liver disease associated with alpha 1-antitrypsin deficiency include decreases in platelets.

### Treatment of Viral-Induced Injuries

Disclosed herein is a method of treating a viral infection-induced liver injury in a subject in need thereof, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof.

Also disclosed herein is a method of decreasing the severity of inflammation in a subject with a viral infection, the method comprising administering a therapeutically effective amount of a hydroxysteroid 17-beta dehydrogenase 13 (HSD17B13) inhibitor to the subject.

Also disclosed herein is a method of decreasing the severity of acute immune response in a subject with a viral infection, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof.

Viral infections are often associated with dysregulated liver inflammatory response. In some embodiments, inactive HSD17B13 is associated with lower levels of genes of activation of immune response including innate immune response, cytokines. Inflammation is a key component of viral-driven injury. In some embodiments, inactive HSD17B13 protects against rapidly progressing fibrosis in HCV patients.

AAV-driven gene therapies are aided by autophagy for incorporating into liver cells so an HSD17B13 inhibitor that improves autophagy and decreases inflammation can both enhance efficacy and decrease side effects.

In some embodiments, the viral infection is hepatitis A. In some embodiments, the viral infection is hepatitis B. In some embodiments, the viral infection is hepatitis C.

In some embodiments, the viral infection is SARS-Cov-2.

### SARS-Cov-2

Liver injury and elevations of liver transaminases are a common feature in patients with severe SARS-Cov-2 infection. There is no apparent increased risk of COVID-19 associated with liver disease, therefore, 2-11% of patients with COVID-19 have underlying liver comorbidities. In contrast, a much larger portion of patients (14-53%) have elevated liver transaminases. Patients suspected of having COVID-19 that test positive for the SARS-CoV-2 RNA had elevations in ALT, AST and decreases in albumin when compared to patients that tested negative for the viral RNA consistent with liver injury. In patients that died from COVID-19, 58% and 78% of patients had liver transaminase elevations. The liver transaminase elevations have been especially notable in patients in intensive care units at 62% compared to 25% of patients not requiring ICU care. Patients with a CT-scan confirmed diagnosis of COVID-19 in the subclinical stage have a lower incidence of liver transaminase elevations when compared to patients diagnosed after the onset of symptoms. While there may be a role of the virus causing direct liver injury as evidenced by the observation of SARS-CoV-2 RNA in blood and feces and 2-10% of patients that have diarrhea, the liver injury may also arise from secondary inflammatory events or the use of many different drugs during critical care.

In addition to liver injury in patients with severe COVID-19, there is an increase in cytokines, or incidence of cytokine storm that is a cause of multiple organ failure and death. To prevent or treat the cytokine storm would therefore be important to treating the patients with the most severe disease. Analysis of COVID-19 patient's blood has shown in general decreases in CD4 and CD8 cells but an increase in Th17 cells. Similarly, in a cohort of patients with severe disease there was an increase in IL-6, IL-10, IL-2 and IFNγ. On the whole, liver injury is seen in COVID-19 patients who are experiencing a broader and more severe disease where multiple organs, beyond the lungs, have become involved. Among the possible reasons for this could be that through an oral route that is then taken up through a more permeable intestinal barrier in patients with co-morbidities such as diabetes. The liver then is exposed to the virus and as a critical mediator for inflammatory responses, could thus mediate either a resolving inflammatory process or mediate an accelerating inflammatory process that leads to increased cytokine release and further organ damage.

The genetic evidence for the gene for the enzyme HSD17B13 association with liver transaminase levels and liver diseases has grown rapidly. At this writing, the genetic evidence includes many studies with genetic and disease information from more than 200,000 individuals. The lack of HSD17B13 activity has been shown to be protective against liver disease caused by over nutrition, exces and chronic alcohol intake, toxins such as copper and viruses such as HCV. The highly reproducible association of HSD17B13 with liver disease, liver transaminase elevations and liver injury in multiple forms has inspired efforts to develop inhibitors of this enzyme to protect against and reverse liver damage. The variety of toxicants and injuries against which HSD17B13 loss of function protects is such that it is proposed here to protect against liver injury in severe COVID-19.

HSD17B13 activity has been shown to be involved in inflammation and inflammatory response. HSD17B13 loss of function has recently been associated with decreased gene expression and protein levels of immune response genes involved in adverse outcomes including IL-6, IL-10 and IL-10. Enzymatically inactive polymorphs of HSD17B13 are associated with lower severity of histopathological endpoints of inflammation in addition to lower ALT levels in patients with nonalcoholic fatty liver disease.

In some embodiments, HSD17B13 inhibitors prevent and treat liver injury and decrease the severity of inflammation and acute immune response in patients with COVID-19.

In some embodiments, the viral infection is associated with a dysregulated liver inflammatory response. In some embodiments, the administration results in lowering the levels of immune response activation genes. In some embodiments, the administration results in decreased inflammation. In some embodiments, the subject in need thereof has elevated levels of alanine aminotransferase (ALT). In some embodiments, the subject in need thereof has elevated levels of aspartate aminotransferase (AST). In some embodiments, the subject in need thereof has decreased levels of albumin.

### Malignancies

Disclosed herein is a method of treating a malignancy selected from hepatocellular carcinoma (HCC), cholangioadenoma, cholangiocarcinoma, gallbladder adenocarcinoma, and malignancy of bile duct, in a subject in need thereof, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof.

Inactive HSD17B13 is associated with lower rates of HCC. However, HSD17B13 expression is low in tumor tissue but normal in the peritumoral space. High protein levels in the peritumoral space is associated with improved survival and tumor free survival. This apparent contradiction supports the use of small molecules to inhibit HSD17B13 but keep the protein available to support autophagic activity. Autophagy is important to maintaining a killer T cell response and use the body's own immune system to fight the tumor. By inhibiting HSD17B13, the surrounding tissue will be capable of killer T cell response due to effective autophagy.

HSD17B13 is highly expressed in normal gallbladder. Inactive HSD17B13 is associated with increased phospotidylcholine which is essential for sequestering cytotoxic bile acids for excretion and bile flow. For these reasons and the additional improved killer T cell response and decreased inflammation and fibrosis are the mechanism by which an inhibitor of HSD17B13 can be used to treat cholangioadenoma and gallbladder adenocarcinoma.

In some embodiments, the malignancy is hepatocellular carcinoma (HCC). In some embodiments, the malignancy is cholangioadenoma. In some embodiments, the malignancy is cholangiocarcinoma. In some embodiments, the malignancy is gallbladder adenocarcinoma. In some embodiments, the HSD17B13 receptor is expressed in the peritumoral space.

### Hemochromatosis

A method of treating hemochromatosis in a subject in need thereof, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof. In some embodiments, the hemochromatosis is primary hemochromatosis. In some embodiments, the hemochromatosis is secondary hemochromatosis. In some embodiments, the hemochromatosis is caused by a liver disease. In some embodiments, the hemochromatosis is drug-induced hemochromatosis.

### Lysosomal Acid Lipase Deficiency (LAL-D)

Disclosed herein is a method of treating lysosomal acid lipase deficiency (LAL-D) in a subject in need thereof, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof. In some embodiments, the lysosomal acid lipase deficiency (LAL-D) is caused by mutations in the LIPA gene. In some embodiments, the lysosomal acid lipase deficiency (LAL-D) causes Wolman disease. In some embodiments, the lysosomal acid lipase deficiency (LAL-D) causes Cholesteryl ester storage disease. In some embodiments, the lysosomal acid lipase deficiency (LAL-D) is caused by mutations in the LIPA gene. In some embodiments, the lysosomal acid lipase deficiency (LAL-D) causes a buildup of fatty substances in the body's cells and tissues.

### Bleeding Disorders/Coagulation Factor Disorders

Disclosed herein is a method of treating a bleeding disorder in a subject in need thereof, the method comprising administering a therapeutically effective amount of ahydroxysteroid 17-beta dehydrogenase 13 (HSD17B13) inhibitor disclosed herein, or a pharmaceutically acceptable salt, solvate, or stereoisomer thereof. In some embodiments, the bleeding disorder is hemophilia A or hemophilia B.

Disclosed herein is a method of treating a coagulation factor disorder in a subject in need thereof, the method comprising administering a therapeutically effective amount of a hydroxysteroid 17-beta dehydrogenase 13 (HSD17B13) inhibitor disclosed herein, or a pharmaceutically acceptable salt, solvate, or stereoisomer thereof. In some embodiments, the coagulation factor disorder is Von Willebrand disease.

In some embodiments, the disease to be treated by inhibition of hydroxysteroid 17-beta dehydrogenase 13 (HSD17B13) is listed in the table below.

| | |
|---|---|
| AAV-induced liver injury | Hepatic adenoma |
| AAV injury | Hepatic encephalopathy |
| Alagille Syndrome | Hepatitis A |
| Alcohol-related liver disease | Hepatitis B |
| Alpha-1 Antitrypsin deficiency | Hepatitis C |
| Autoimmune hepatitis | Hepatorenal syndrome |
| Biliary atresia | Intrahepatic cholestasis |
| | of pregnancy (ICP) |
| Cholangiocarcinoma | Liver cancer |
| Covid 19 related | LAL-D |
| Crigler-Najjar Syndrome | Newborn jaundice |
| Cystic fibrosis - related | NAFLD |
| Drug induced liver injury | PBC |
| Galactosemia | PSC |
| Gallbladder cancer | Progressive familial |
| | intrahepatic cholestasis |
| Gilbert syndrome | Reye syndrome |
| HCC | Type I glycogen storage disease |
| Hemochromatosis | Wilson disease |

### Dosing

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of or risk factor for the disease being treated and is currently in remission, a pharmaceutical composition comprising a compound described herein, or a pharmaceutically acceptable salt thereof, in order to prevent a return of the symptoms of the disease or condition. In one aspect, prophylactic treatments include administering to a mammal having patatin-like phospholipase domain-containing 3 (PNPLA3) polymorphism, a pharmaceutical composition comprising a compound described herein, or a pharmaceutically acceptable salt thereof, in order to prevent liver damages. The 148 Isoleucine to Methionine protein variant (I148M) of patatin-like phospholipase domain-containing 3 (PNPLA3), a protein is expressed in the liver and is involved in lipid metabolism, has recently been identified as a major determinant of liver fat content. Several studies confirmed that the I148M variant predisposes towards the full spectrum of liver damage associated with fatty liver: from simple steatosis to steatohepatitis and progressive fibrosis. Furthermore, the I148M variant represents a major determinant of progression of alcohol related steatohepatitis to cirrhosis, and to influence fibrogenesis and related clinical outcomes in chronic hepatitis C virus hepatitis, and possibly chronic hepatitis B virus hepatitis, hereditary hemochromatosis and primary sclerosing cholangitis. In some embodiments, PNPLA3 polymorphism is used to predict liver disease progression.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage, or the frequency of administration, or both, is reduced, as a function of the symptoms, to a level at which the improved disease, disorder or condition is retained. In certain embodiments, however, the patient requires intermittent or daily treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage, or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₁₀ and the ED₉₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In one embodiment, the compounds of this invention may be administered to animals. The compounds can be administered orally or parenterally, including the intravenous, intramuscular, intraperitoneal, subcutaneous, rectal, and topical routes of administration.

In another aspect, provided herein are pharmaceutical compositions comprising a compound described herein, or a pharmaceutically acceptable salt, solvate, or stereoisomer thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including compounds described herein, or a pharmaceutically acceptable salt, solvate, or stereoisomer thereof are manufactured in a conventional manner, such as, by way of example only, by means of conventional mixing, dissolving, granulating, dragee-making, levigating, emulsifying, encapsulating, entrapping, or compression processes.

Pharmaceutical compositions for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical compositions that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

Pharmaceutical compositions for parental use are formulated as infusions or injections. In some embodiments, the pharmaceutical composition suitable for injection or infusion includes sterile aqueous solutions, or dispersions, or sterile powders comprising a compound described herein, or a pharmaceutically acceptable salt, solvate, or stereoisomer thereof. In some embodiments, the pharmaceutical composition comprises a liquid carrier. In some embodiments, the liquid carrier is a solvent or liquid dispersion medium comprising, for example, water, saline, ethanol, a polyol (for example, glycerol, propylene glycol, liquid polyethylene glycols, and the like), vegetable oils, nontoxic glyceryl esters, and any combinations thereof. In some embodiments, the pharmaceutical compositions further comprise a preservative to prevent growth of microorganisms.

### Combination

Disclosed herein are method of treating a liver disease, metabolic disease, or cardiovascular disease using a compound disclosed herein, or a pharmaceutically acceptable salt, solvate, or stereoisomer thereof, in combination with an additional therapeutic agent.

In some embodiments, the additional therapeutic agent is used for the treatment of diabetes or diabetes related disorder or conditions.

In some instances, the additional therapeutic agent comprises a statin, an insulin sensitizing drug, an insulin secretagogue, an alpha-glucosidase inhibitor, a GLP agonist, a GIP agonist, a THR beta agonist, a PDE inhibitor, a DPP-4 inhibitor (such as sitagliptin, vildagliptin, saxagliptin, linagliptin, anagliptin, teneligliptin, alogliptin, gemigliptin, or dutogliptin), a catecholamine (such as epinephrine, norepinephrine, or dopamine), peroxisome proliferator-activated receptor (PPAR)-gamma agonist (e.g., a thiazolidinedione (TZD) [such as pioglitazone, rosiglitazone, rivoglitazone, or troglitazone], aleglitazar, farglitazar, muraglitazar, or tesaglitazar), peroxisome proliferator-activated receptor (PPAR)-alpha agonist, peroxisome proliferator-activated receptor (PPAR)-delta agonist, a farnesoid X receptor (FXR) agonist (e.g., obeticholic acid), or a combination thereof. In some cases, the statin is a HMG-CoA reductase inhibitor. In other instances, additional therapeutic agents include fish oil, fibrate, vitamins such as niacin, retinoic acid (e.g., 9 cis-retinoic acid), nicotinamide ribonucleoside or its analogs thereof, or combinations thereof. In other instances, additional therapeutic agents include ACC inhibitors, FGF19 and FGF21 mimics, CCR2/CCR5 antagonists, or combinations thereof.

In some embodiments, the additional therapeutic agent is vivitrol.

In some embodiments, the additional therapeutic agent is a statin such as a HMG-CoA reductase inhibitor, fish oil, fibrate, niacin, or a combination thereof. In other instances, the additional therapeutic agent is a dyslipidemia drug that prevent lipid absorption such as orlistat.

In some embodiments, the additional therapeutic agent is a vitamin such as retinoic acid or tocopheryl acetate for the treatment of diabetes and diabetes related disorder or condition such as lowering elevated body weight and/or lowering elevated blood glucose from food intake.

In some embodiments, the additional therapeutic agent is a glucose-lowering agent. In some embodiments, the additional therapeutic agent is an anti-obesity agent. In some embodiments, the additional therapeutic agent is selected from among a peroxisome proliferator activated receptor (PPAR) agonist (gamma, dual, or pan), a dipeptidyl peptidase (IV) inhibitor, a glucagon-like peptide-1 (GLP-I) analog, insulin or an insulin analog, an insulin secretagogue, a sodium glucose co-transporter 2 (SGLT2) inhibitor, a glucophage, a human amylin analog, a biguanide, an alpha-glucosidase inhibitor, a meglitinide, a thiazolidinedione, and sulfonylurea. In some embodiments, the additional therapeutic agent is metformin, sitagliptin, saxaglitpin, repaglinide, nateglinide, exenatide, liraglutide, insulin lispro, insulin aspart, insulin glargine, insulin detemir, insulin isophane, and glucagon-like peptide 1, or any combination thereof. In some embodiments, the additional therapeutic agent is a lipid-lowering agent.

In some embodiments, the additional therapeutic agent is an antioxidant, corticosteroid such as budesonide, anti-tumor necrosis factor (TNF), or a combination thereof.

In some embodiments, the additional therapeutic agent is administered at the same time as the compound disclosed herein. In some embodiments, the additional therapeutic agent and the compound disclosed herein are administered sequentially. In some embodiments, the additional therapeutic agent is administered less frequently than the compound disclosed herein. In some embodiments, the additional therapeutic agent is administered more frequently than the compound disclosed herein. In some embodiments, the additional therapeutic agent is administered prior than the administration of the compound disclosed herein. In some embodiments, the additional therapeutic agent is administered after the administration of the compound disclosed herein.

### Compound Synthesis

Embodiments of compounds of Formula (I) can be prepared according to general reaction scheme 1, where X¹, X², X³, Y¹, and Y² are reactive functional groups (e.g., F, Cl, Br, OH, aldehyde, ketone, boronic acid/ester, acid, amine, hydrazine, etc.) selected based on compatibility with overall reaction scheme and desired reaction selectivity and position. PG represents protecting group (e.g., THP, Me, Bn, Boc, etc.), the use of which are well known. As depicted in Scheme 1, compound A-1 is either purchased as a commercial reagent or prepared according to known methods and is reacted with an appropriate nucleophile (e.g., A-2 wherein Y¹ is a hydrazine will react with A-1 wherein X¹ is CHO and X² is F) to form A-3. The desired -L-R² substituent may then be added via a Suzuki coupling or simple displacement reaction (e.g., wherein Y² is an amine and X³ is a Br) to give A-4. Removal of the protecting group (e.g., Me- is removed with BBr3) to yield desired compound of Formula (I). Alternatively, scheme 1 can be modified at any step to add and/or modify a substituent.

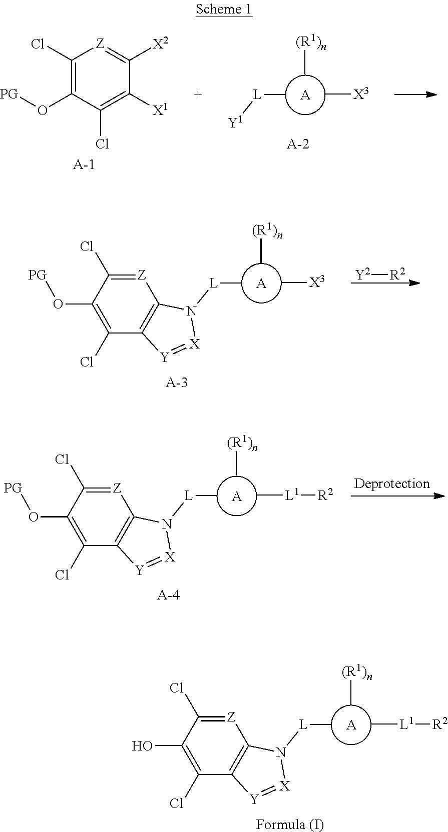

Embodiments of compounds of Formula (II) can be prepared according to general reaction scheme 2, where X¹, X², Y¹ and Y² are reactive functional groups (e.g., F, Cl, Br, OH, aldehyde, ketone, boronic acid/ester, acid, amine, etc.) selected based on compatibility with overall reaction scheme and desired reaction selectivity and position. PG represents protecting group (e.g., Boc, Fmoc, SEM, THP, Me, Bn, etc.), the use of which are well known. As depicted in Scheme 2, compound A-5 is either purchased as a commercial reagent or prepared according to known methods and is reacted with an appropriate nucleophile under suitable conditions (e.g., Y¹—R² wherein Y¹ is an amine and X¹ is Cl) to form A-6. A-8 can be achieved via a Suzuki coupling or simple displacement reaction between A-6 and A-7 (e.g., A7 wherein Y² is boronic acid/ester and X² is an Iodide). Removal of the protecting groups will yield the desired compound of Formula (II). Alternatively, scheme 2 can be modified at any step to add and/or modify a substituent.

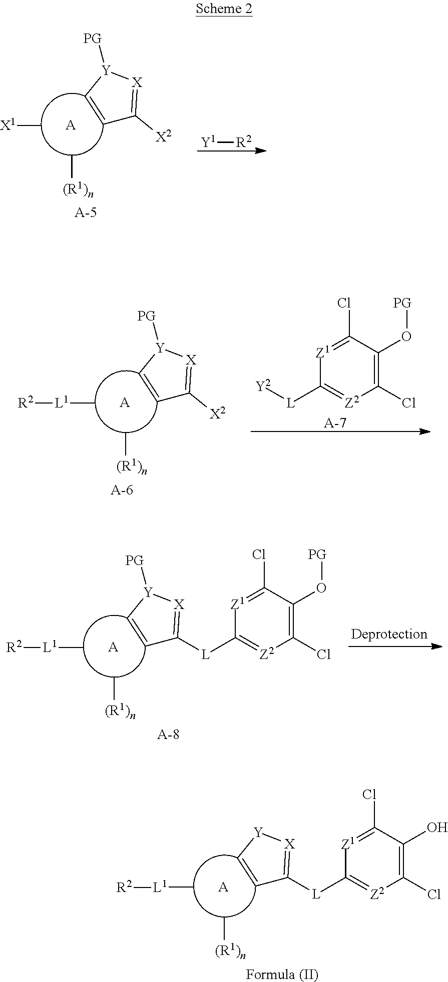

Embodiments of compounds of Formula (III) can be prepared according to general reaction scheme 3, where X¹ and Y¹ are reactive functional groups (e.g., F, Cl, Br, OH, aldehyde, ketone, boronic acid/ester, acid, acyl chloride, amine, hydrazine, etc.) selected based on compatibility with overall reaction scheme and desired reaction selectivity and position. PG represents protecting group (e.g., THP, Me, Bn, Boc, etc.), the use of which are well known. As depicted in Scheme 3, compound A-9 is either purchased as a commercial reagent or prepared according to known methods and is reacted with an appropriate acyl chloride A-7 under suitable cyclization conditions to form fused oxazole A-10. The desired -L-R² substituent may then be added via a suitable reaction e.g., Suzuki coupling or simple displacement reaction to give A-11. Removal of the protecting group yield desired compound of Formula (III). Alternatively, scheme 3 can be modified at any step to add and/or modify a substituent.

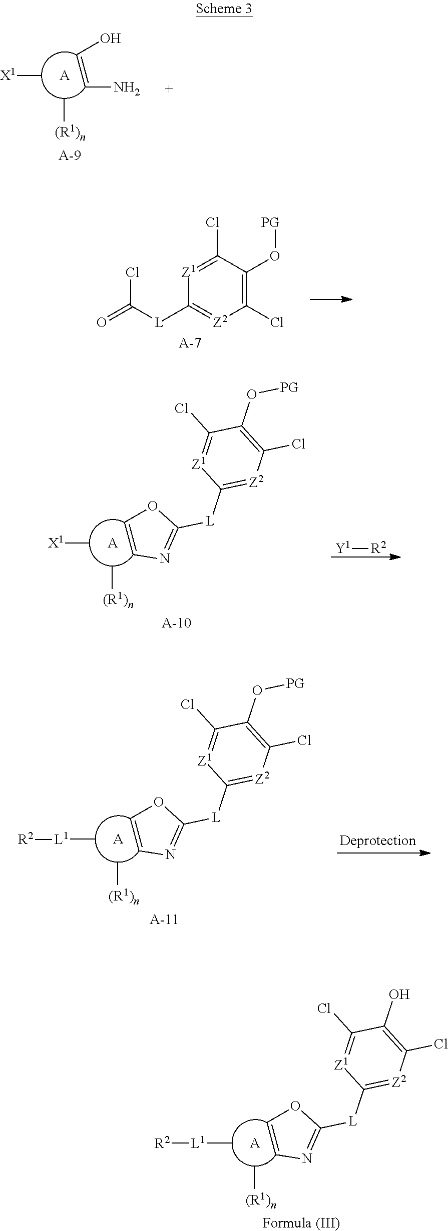

Embodiments of compounds of Formula (IV) can be prepared according to general reaction scheme 4, where X¹, Y¹ and Y² are reactive functional groups (e.g., F, Cl, Br, OH, aldehyde, ketone, boronic acid/ester, acid, amine, etc.) selected based on compatibility with overall reaction scheme and desired reaction selectivity and position. PG represents protecting group (e.g., Boc, Fmoc, SEM, THP, Me, Bn, etc.), the use of which are well known. As depicted in Scheme 4, compound A-12 is either purchased as a commercial reagent or prepared according to known methods and is reacted with an appropriate nucleophile under suitable conditions (e.g., Y¹—R² wherein Y¹ is an amine and X¹ is Cl) to form A-13. Deprotection of A-13 yields A-14 which can react with A-7 via a displacement reaction (e.g., A7 wherein Y² is Cl). Removal of the protecting groups will yield the desired compound of Formula (IV). Alternatively, scheme 4 can be modified at any step to add and/or modify a substituent.

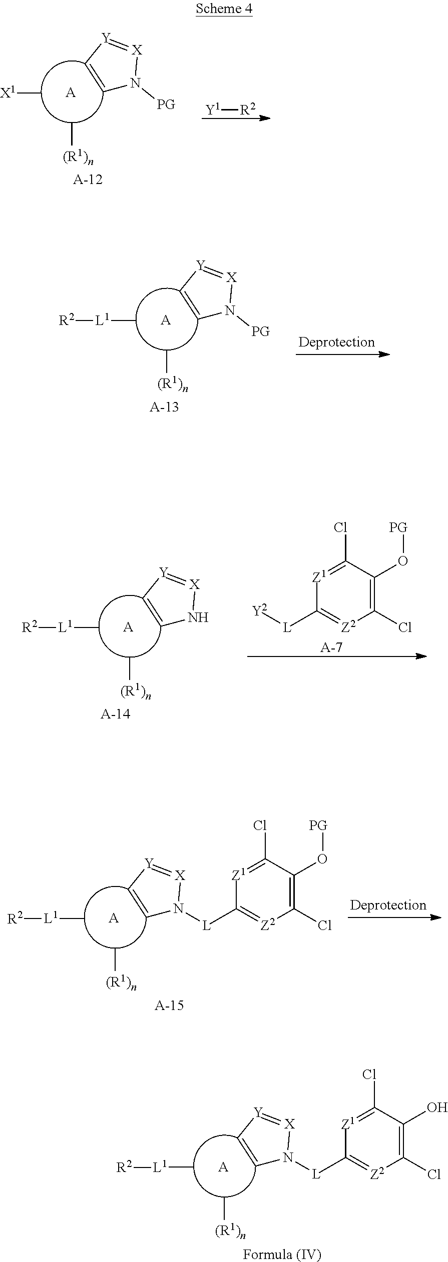

Embodiments of compounds of Formula (V) can be prepared according to general reaction scheme 5, where X¹, X² and Y¹ are reactive functional groups (e.g., F, Cl, Br, OH, NH₂, CHO, ketone, boronic acid/ester, acid, acyl chloride, hydrazine, etc.) selected based on compatibility with overall reaction scheme and desired reaction selectivity and position. PG represents protecting group (e.g., THP, Me, Bn, Boc, etc.), the use of which are well known. As depicted in Scheme 5, compound A-16 is either purchased as a commercial reagent or prepared according to known methods and is reacted with an appropriate nucleophile A-7 under suitable cyclization conditions (e.g., A-16 wherein X¹ is NH₂; X² is CHO and A-7 wherein Y¹ is NH₂) to form fused ring A-17. Removal of the protecting group yield desired compound of Formula (V). Alternatively, scheme 5 can be modified at any step to add and/or modify a substituent.

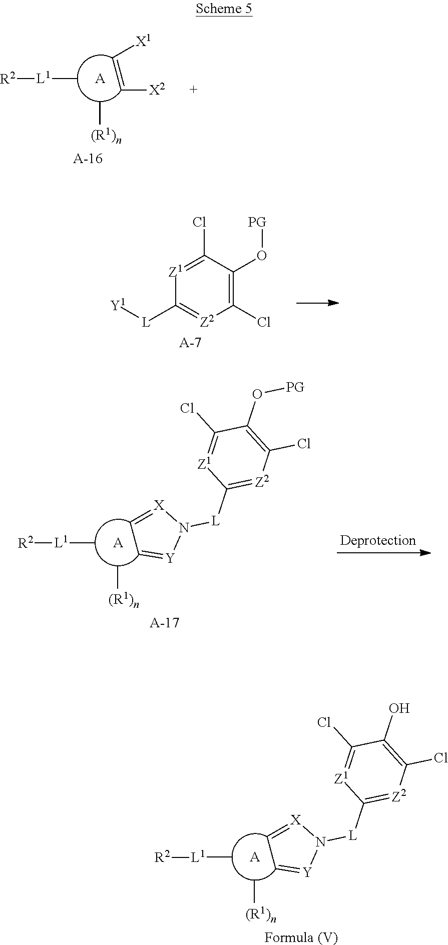

Embodiments of compounds of Formula (VI) can be prepared according to general reaction scheme 6, where X¹ is a reactive functional group (e.g., F, Cl, Br, aldehyde, acid, acyl chloride, etc.) selected based on compatibility with overall reaction scheme and desired reaction selectivity and position. PG represents protecting group (e.g., THP, Me, Bn, Boc, etc.), the use of which are well known. As depicted in Scheme 6, an electrophile, A-19 (e.g., wherein X¹ is Cl) is either purchased as a commercial reagent or prepared according to known methods and is reacted with oxazolidine-2,4-dione or a thiazolidine-2,4-dione, A-18 to form A-20. Intermediate compounds A-22 and A-23 can be achieved by the condensation reaction between A-20 and a corresponding aldehyde (e.g., A-7 for A-22 and A-21 for A-23). Removal of the protecting group yield desired compounds of Formula (VIa) and of Formula (VIb). Alternatively, scheme 6 can be modified at any step to add and/or modify a substituent.

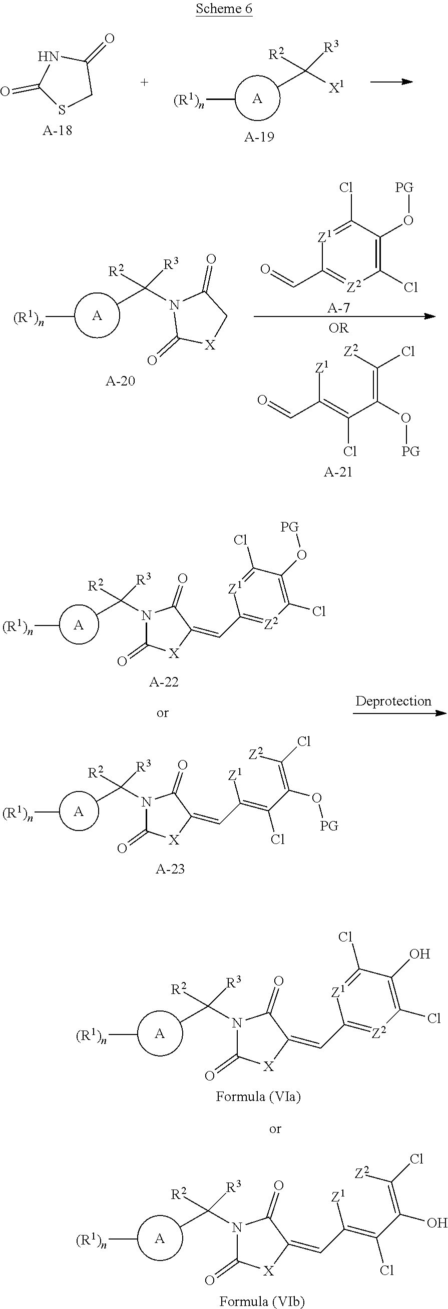

Embodiments of compounds of Formula (VII) can be prepared according to general reaction scheme 7, where X¹, X², X³ and Y¹ are reactive functional groups (e.g., F, Cl, Br, OH, aldehyde, ketone, boronic acid/ester, acid, amine, hydrazine, etc.) selected based on compatibility with overall reaction scheme and desired reaction selectivity and position. PG represents protecting group (e.g., THP, Me, Bn, Boc, etc.), the use of which are well known. As depicted in Scheme 7, compound A-24 either purchased as a commercial reagent or prepared according to known methods is reacted with an appropriate nucleophile (e.g., A-24 wherein X¹ is —NH₂; X² is —(R¹)ₙNH will react with A-25 wherein Y1 is —CHO) to form A-26. A transformation of X³ to the desired carboxylate functionality can be achieved (e.g., by hydrolysis where X³ is an ester) to give A-27. Standard amide formation with appropriate amine and removal of the protecting group yields desired compounds of Formula (VII). Alternatively, scheme 7 can be modified at any step to add and/or modify a substituent.

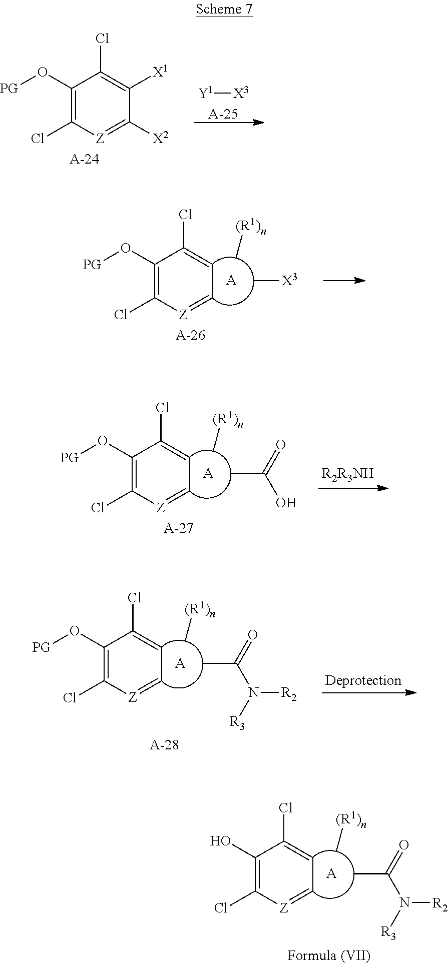

What is claimed is:

1. A compound of Formula (I), or a pharmaceutically acceptable salt, solvate, or stereoisomer thereof: 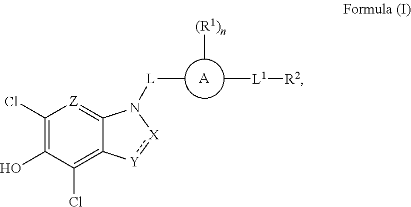
wherein:
Z is N or CR^{Z};
R^{Z} is hydrogen, deuterium, halogen, —CN, —OH, —OR^{a}, —NR^{c}R^{d}, or C₁-C₆alkyl optionally substituted with one or more R; [symbol]
is a single bond;
X is —C(R⁴)₂— or —C(═NR³)—;
Y is —O—, —NR³—, or —C(R⁴)₂—;
or [symbol]
is a double bond;
X is —N— or —CR⁵—;
Y is —N— or —CR⁵—;
R³ is hydrogen or C₁-C₆alkyl optionally substituted with one or more R;
each R⁴ is independently hydrogen, deuterium, halogen, —CN, —OH, —OR^{a}, —NR^{c}R^{d}, or C₁-C₆alkyl optionally substituted with one or more R;
or two R⁴ are taken together to form an oxo;
each R⁵ is independently hydrogen, deuterium, halogen, —CN, —OH, —OR^{a}, —NR^{c}R^{d}, or C₁-C₆alkyl optionally substituted with one or more R;
L is absent, —C(R⁷)₂—, —C(═O)—, —S(═O)—, —S(═O)₂—, —C(R)₂—NR⁶—, or —C(═O)—NR⁶—;
R⁶ is hydrogen or C₁-C₆alkyl optionally substituted with one or more R;
each R⁷ is independently hydrogen, deuterium, halogen, —CN, —OH, —OR^{a}, —NR^{c}R^{d}, or C₁-C₆alkyl optionally substituted with one or more R;
or two R⁷ are taken together to form an oxo;
or two R⁷ are taken together to form a cycloalkyl or heterocycloalkyl; each optionally substituted with one or more R;
Ring A is a cycloalkyl, heterocycloalkyl, aryl, or heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R¹ on the same atom are taken together to form an oxo;
n is 0-4;
L¹ is absent, —O—, —S—, —NR⁶—, —C(═O)—, —C(═O)O—, —S(═O)—, —S(═O)₂—, —NR⁶—C(R⁷)₂—, —C(R⁷)₂—NR⁶—, —NR⁶—C(═O)—, or —C(═O)—NR⁶—;
R² is C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein the alkyl, alkenyl, alkynyl, cycloalkyl, heterocycloalkyl, aryl, and heteroaryl is optionally substituted with one or more R^{2a};
each R^{2a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{2a} on the same atom are taken together to form an oxo;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

2. The compound of claim 1, or a pharmaceutically acceptable salt, solvate, or stereoisomer thereof, wherein the compound is of Formula (Ia):

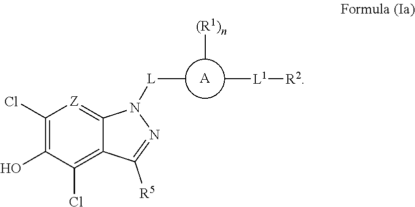

3. The compound of claim 1, or a pharmaceutically acceptable salt, solvate, or stereoisomer thereof, wherein the compound is of Formula (Ib):

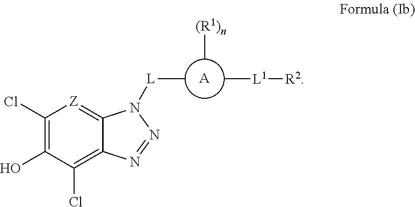

4. The compound of claim 1, or a pharmaceutically acceptable salt, solvate, or stereoisomer thereof, wherein the compound is of Formula (Ic):

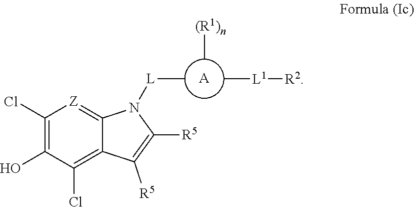

5. The compound of claim 1, or a pharmaceutically acceptable salt, solvate, or stereoisomer thereof, wherein the compound is of Formula (Id):

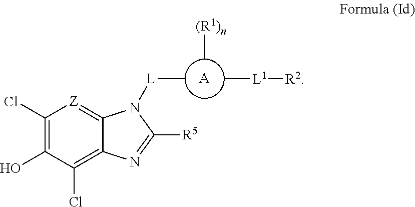

6. The compound of claim 1, or a pharmaceutically acceptable salt, solvate, or stereoisomer thereof, wherein the compound is of Formula (Ie):

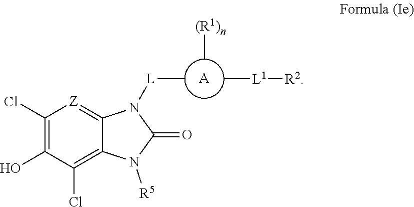

7. The compound of claim 1, or a pharmaceutically acceptable salt, solvate, or stereoisomer thereof, wherein the compound is of Formula (If):

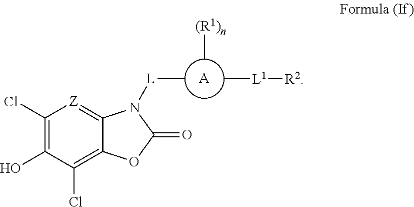

8. The compound of claim 1, or a pharmaceutically acceptable salt, solvate, or stereoisomer thereof, wherein the compound is of Formula (Ig):

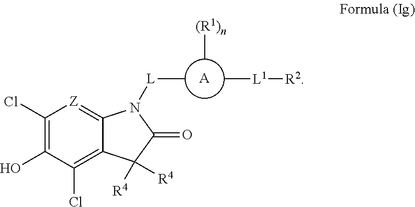

9. The compound of any one of claims 1-8, or a pharmaceutically acceptable salt, solvate, or stereoisomer thereof, wherein R³ is hydrogen.

10. The compound of any one of claims 1-9, or a pharmaceutically acceptable salt, solvate, or stereoisomer thereof, wherein each R⁴ is hydrogen.

11. The compound of any one of claims 1-9, or a pharmaceutically acceptable salt, solvate, or stereoisomer thereof, wherein two R⁴ are taken together to form an oxo.

12. The compound of any one of claims 1-11, or a pharmaceutically acceptable salt, solvate, or stereoisomer thereof, wherein each R⁵ is hydrogen.

13. The compound of any one of claims 1-12, or a pharmaceutically acceptable salt, solvate, or stereoisomer thereof, wherein R^{Z} is hydrogen.

14. The compound of any one of claims 1-13, or a pharmaceutically acceptable salt, solvate, or stereoisomer thereof, wherein R⁶ is hydrogen.

15. The compound of any one of claims 1-14, or a pharmaceutically acceptable salt, solvate, or stereoisomer thereof, wherein each R⁷ is hydrogen.

16. The compound of any one of claims 1-15, or a pharmaceutically acceptable salt, solvate, or stereoisomer thereof, wherein Ring A is a aryl or heteroaryl.

17. The compound of any one of claims 1-15, or a pharmaceutically acceptable salt, solvate, or stereoisomer thereof, wherein Ring A is a phenyl.

18. The compound of any one of claims 1-15, or a pharmaceutically acceptable salt, solvate, or stereoisomer thereof, wherein Ring A is a 5- or 6-membered heteroaryl.

19. The compound of any one of claims 1-15, or a pharmaceutically acceptable salt, solvate, or stereoisomer thereof, wherein Ring A is a 6-membered heteroaryl.

20. The compound of any one of claims 1-19, or a pharmaceutically acceptable salt, solvate, or stereoisomer thereof, wherein each R¹ is independently deuterium, halogen, C₁-C₆alkyl, or C₁-C₆haloalkyl.

21. The compound of any one of claims 1-19, or a pharmaceutically acceptable salt, solvate, or stereoisomer thereof, wherein each R¹ is independently halogen or C₁-C₆alkyl.

22. The compound of any one of claims 1-21, or a pharmaceutically acceptable salt, solvate, or stereoisomer thereof, wherein n is 0 or 1.

23. The compound of any one of claims 1-22, or a pharmaceutically acceptable salt, solvate, or stereoisomer thereof, wherein R² is aryl or heteroaryl; wherein the aryl and heteroaryl is optionally substituted with one or more R^{2a}.

24. The compound of any one of claims 1-22, or a pharmaceutically acceptable salt, solvate, or stereoisomer thereof, wherein R² is cycloalkyl or heterocycloalkyl; wherein the cycloalkyl and heterocycloalkyl is optionally substituted with one or more R^{2a}.

25. The compound of any one of claims 1-24, or a pharmaceutically acceptable salt, solvate, or stereoisomer thereof, wherein each R^{2a} is independently deuterium, halogen, —CN, —OH, —OR^{a}, —S(═O)₂R^{a}, —NR^{c}R^{d}, C₁-C₆alkyl, or C₁-C₆haloalkyl.

26. A compound of Formula (II), or a pharmaceutically acceptable salt, solvate, or stereoisomer thereof: 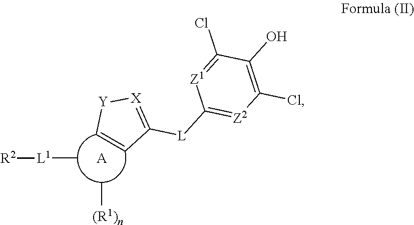
wherein:
Ring A is phenyl or 6-membered heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
n is 0-3;
L¹ is absent, —O—, —S—, —NR⁶—, —C(═O)—, —C(═O)O—, —S(═O)—, —S(═O)₂—, —NR⁶—C(R⁷)₂—, —C(R⁷)₂—NR⁶—, —NR⁶—C(═O)—, or —C(═O)—NR⁶—;
R² is cycloalkyl, heterocycloalkyl, aryl, or heteroaryl; wherein the cycloalkyl, heterocycloalkyl, aryl, and heteroaryl is independently optionally substituted with one or more R^{2a};
each R^{2a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{2a} on the same atom are taken together to form an oxo;
X is —N— or —CR³—;
Y is —O—, —NR⁴—, or —C(R⁵)₂—;
R³ is hydrogen, deuterium, halogen, —CN, —OH, —OR^{a}, —NR^{c}R^{d}, or C₁-C₆alkyl optionally substituted with one or more R;
R⁴ is hydrogen or C₁-C₆alkyl optionally substituted with one or more R;
each R⁵ is independently hydrogen, deuterium, halogen, —CN, —OH, —OR^{a}, —NR^{c}R^{d}, or C₁-C₆alkyl optionally substituted with one or more R;
or two R⁵ are taken together to form an oxo;
L is absent, —O—, —S—, —NR⁶—, —C(R⁷)₂—, —C(═O)—, —S(═O)—, —S(═O)₂—, —NR⁶—C(R)₂—, —C(R⁷)₂—NR⁶—, —NR⁶—C(═O)—, —C(═O)—NR⁶—, or —NR⁶—C(═O)—NR⁶—;
R⁶ is hydrogen or C₁-C₆alkyl optionally substituted with one or more R;
each R⁷ is independently hydrogen, deuterium, halogen, —CN, —OH, —OR^{a}, —NR^{c}R^{d}, or C₁-C₆alkyl optionally substituted with one or more R;
or two R⁷ are taken together to form an oxo;
or two R⁷ are taken together to form a cycloalkyl or heterocycloalkyl; each optionally substituted with one or more R;
Z¹ is N or CR^{Z1};
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z2};
R^{Z2} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo;
provided that the compound is not:

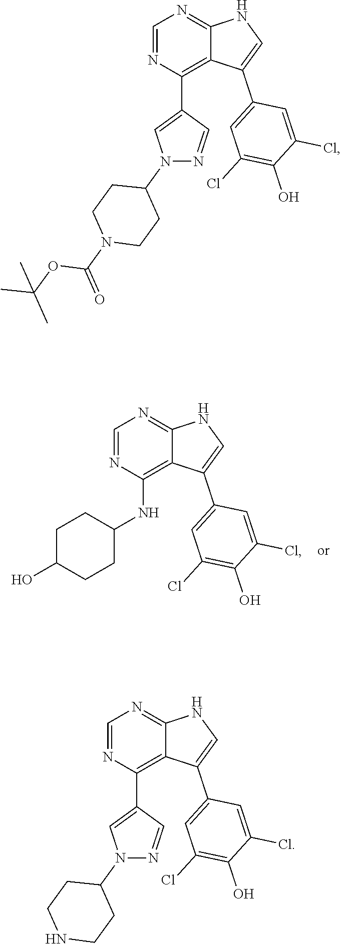 [SMILES: CC(C)(C)OC(=O)N1CCC(n2cc(-c3ncnc4[nH]cc(-c5cc(Cl)c(O)c(Cl)c5)c34)cn2)CC1 ; Oc1c(Cl)cc(-c2c[nH]c3ncnc(NC4CCC(O)CC4)c23)cc1Cl ; Oc1c(Cl)cc(-c2c[nH]c3ncnc(-c4cnn(C5CCNCC5)c4)c23)cc1Cl]

27. The compound of claim 26, or a pharmaceutically acceptable salt, solvate, or stereoisomer thereof, wherein the compound is of Formula (IIa): 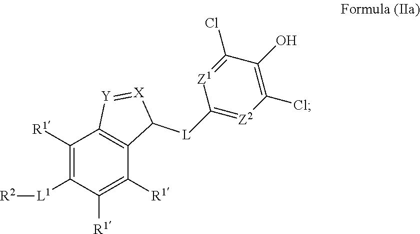
wherein each R^{1′} is independently hydrogen or R¹.

28. The compound of claim 26, or a pharmaceutically acceptable salt, solvate, or stereoisomer thereof, wherein the compound is of Formula (IIb): 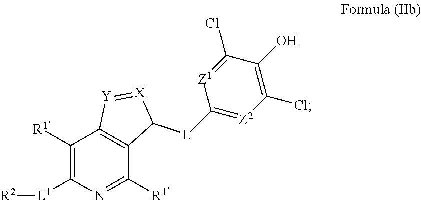
wherein each R^{1′} is independently hydrogen or R¹.

29. The compound of any one of claims 26-28, or a pharmaceutically acceptable salt, solvate, or stereoisomer thereof, wherein R³ is hydrogen.

30. The compound of any one of claims 26-29, or a pharmaceutically acceptable salt, solvate, or stereoisomer thereof, wherein R⁴ is hydrogen.

31. The compound of any one of claims 26-30, or a pharmaceutically acceptable salt, solvate, or stereoisomer thereof, wherein each R⁵ is independently hydrogen or C₁-C₆alkyl.

32. The compound of any one of claims 26-31, or a pharmaceutically acceptable salt, solvate, or stereoisomer thereof, wherein each R¹ is independently deuterium, halogen, C₁-C₆alkyl, or C₁-C₆haloalkyl.

33. The compound of any one of claims 26-32, or a pharmaceutically acceptable salt, solvate, or stereoisomer thereof, wherein n is 0 or 1.

34. The compound of any one of claims 26-33, or a pharmaceutically acceptable salt, solvate, or stereoisomer thereof, wherein R² is cycloalkyl or heterocycloalkyl; wherein the cycloalkyl and heterocycloalkyl is independently optionally substituted with one or more R^{2a}.

35. The compound of any one of claims 26-33, or a pharmaceutically acceptable salt, solvate, or stereoisomer thereof, wherein R² is aryl or heteroaryl; wherein the aryl and heteroaryl is independently optionally substituted with one or more R^{2a}.

36. The compound of any one of claims 26-35, or a pharmaceutically acceptable salt, solvate, or stereoisomer thereof, wherein each R^{2a} is independently deuterium, halogen, —CN, —OH, —OR^{a}, —S(═O)₂R^{a}, —NR^{c}R^{d}, C₁-C₆alkyl, or C₁-C₆haloalkyl.

37. The compound of any one of claims 26-36, or a pharmaceutically acceptable salt, solvate, or stereoisomer thereof, wherein R⁶ is hydrogen.

38. The compound of any one of claims 26-37, or a pharmaceutically acceptable salt, solvate, or stereoisomer thereof, wherein each R⁷ is independently hydrogen, halogen, or C₁-C₆alkyl.

39. The compound of any one of claims 26-38, or a pharmaceutically acceptable salt, solvate, or stereoisomer thereof, wherein R^{Z1} is hydrogen.

40. The compound of any one of claims 26-39, or a pharmaceutically acceptable salt, solvate, or stereoisomer thereof, wherein R^{Z2} is hydrogen.

41. A compound of Formula (II), or a pharmaceutically acceptable salt, solvate, or stereoisomer thereof: 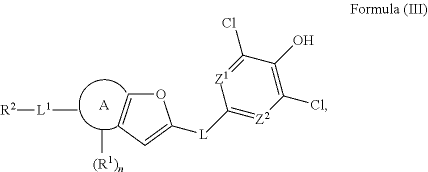
wherein:
Ring A is aryl or 6-membered heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
n is 0-3;
L¹ is absent, —O—, —S—, —NR⁶—, —C(═O)—, —C(═O)O—, —S(═O)—, —S(═O)₂—, —NR⁶—C(R⁷)₂—, —C(R⁷)₂—NR⁶—, —NR⁶—C(═O)—, or —C(═O)—NR⁶—;
R² is cycloalkyl, heterocycloalkyl, aryl, or heteroaryl; wherein the cycloalkyl, heterocycloalkyl, aryl, and heteroaryl is independently optionally substituted with one or more R^{2a};
each R^{2a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{2a} on the same atom are taken together to form an oxo;
L is absent, —O—, —S—, —NR⁶—, —C(R⁷)₂—, —C(═O)—, —S(═O)—, —S(═O)₂—, —NR⁶—C(R)₂—, —C(R⁷)₂—NR⁶—, —NR⁶—C(═O)—, —C(═O)—NR⁶—, or —NR⁶—C(═O)—NR⁶—;
R⁶ is hydrogen or C₁-C₆alkyl optionally substituted with one or more R;
each R⁷ is independently hydrogen, deuterium, halogen, —CN, —OH, —OR^{a}, —NR^{c}R^{d}, or C₁-C₆alkyl optionally substituted with one or more R;
or two R⁷ are taken together to form an oxo;
Z¹ is N or CR^{Z1};
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z2};
R^{Z2} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

42. The compound of claim 41, or a pharmaceutically acceptable salt, solvate, or stereoisomer thereof, wherein each R¹ is independently deuterium, halogen, C₁-C₆alkyl, or C₁-C₆haloalkyl.

43. The compound of claim 41 or 42, or a pharmaceutically acceptable salt, solvate, or stereoisomer thereof, wherein n is 0 or 1.

44. The compound of any one of claims 41-43, or a pharmaceutically acceptable salt, solvate, or stereoisomer thereof, wherein R² is cycloalkyl or heterocycloalkyl; wherein the cycloalkyl and heterocycloalkyl is independently optionally substituted with one or more R^{2a}.

45. The compound of any one of claims 41-43, or a pharmaceutically acceptable salt, solvate, or stereoisomer thereof, wherein R² is aryl or heteroaryl; wherein the aryl and heteroaryl is independently optionally substituted with one or more R^{2a}.

46. The compound of any one of claims 41-45, or a pharmaceutically acceptable salt, solvate, or stereoisomer thereof, wherein each R^{2a} is independently deuterium, halogen, —CN, —OH, —OR^{a}, —S(═O)₂R^{a}, —NR^{c}R^{d}, C₁-C₆alkyl, or C₁-C₆haloalkyl.

47. The compound of any one of claims 41-46, or a pharmaceutically acceptable salt, solvate, or stereoisomer thereof, wherein R⁶ is hydrogen.

48. The compound of any one of claims 41-47, or a pharmaceutically acceptable salt, solvate, or stereoisomer thereof, wherein each R⁷ is independently hydrogen, halogen, or C₁-C₆alkyl.

49. The compound of any one of claims 41-48, or a pharmaceutically acceptable salt, solvate, or stereoisomer thereof, wherein R^{Z1} is hydrogen.

50. The compound of any one of claims 41-49, or a pharmaceutically acceptable salt, solvate, or stereoisomer thereof, wherein R^{Z2} is hydrogen.

51. A compound of Formula (IV), or a pharmaceutically acceptable salt, solvate, or stereoisomer thereof: 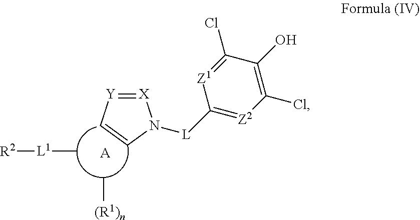
wherein:
Ring A is phenyl or 6-membered heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
n is 0-3;
L¹ is absent, —O—, —S—, —NR⁶—, —C(═O)—, —C(═O)O—, —S(═O)—, —S(═O)₂—, —NR⁶—C(R⁷)₂—, —C(R⁷)₂—NR⁶—, —NR⁶—C(═O)—, or —C(═O)—NR⁶—;
R² is cycloalkyl, heterocycloalkyl, aryl, or heteroaryl; wherein the cycloalkyl, heterocycloalkyl, aryl, and heteroaryl is optionally substituted with one or more R^{2a};
each R^{2a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{2a} on the same atom are taken together to form an oxo;
X is —N— or —CR^{X}—;
R^{X} is hydrogen, deuterium, halogen, —CN, —OH, —OR^{a}, —NR^{c}R^{d}, or C₁-C₆alkyl optionally substituted with one or more R;
Y is —N— or —CR^{Y}—;
R^{Y} is hydrogen, deuterium, halogen, —CN, —OH, —OR^{a}, —NR^{c}R^{d}, or C₁-C₆alkyl optionally substituted with one or more R;
L is absent, —C(R⁷)₂—, —C(═O)—, —S(═O)—, —S(═O)₂—, —C(R)₂—NR⁶—, or —C(═O)—NR⁶—;
R⁶ is hydrogen or C₁-C₆alkyl optionally substituted with one or more R;
each R⁷ is independently hydrogen, deuterium, halogen, —CN, —OH, —OR^{a}, —NR^{c}R^{d}, or C₁-C₆alkyl optionally substituted with one or more R;
or two R⁷ are taken together to form an oxo;
or two R⁷ are taken together to form a cycloalkyl or heterocycloalkyl; each optionally substituted with one or more R;
Z¹ is N or CR^{Z1};
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z2};
R^{Z2} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

52. The compound of claim 51, or a pharmaceutically acceptable salt, solvate, or stereoisomer thereof, wherein the compound is of Formula (IVa): 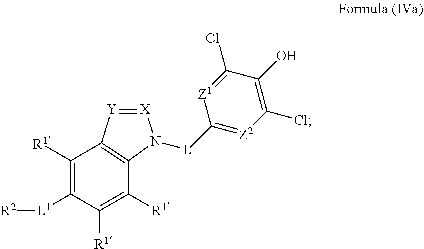
wherein each R^{1′} is independently hydrogen or R¹.

53. The compound of claim 51, or a pharmaceutically acceptable salt, solvate, or stereoisomer thereof, wherein the compound is of Formula (IVb): 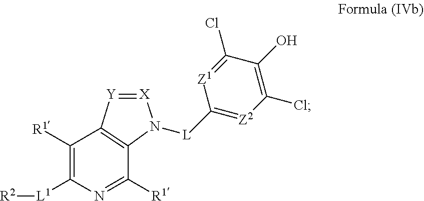
wherein each R^{1′} is independently hydrogen or R¹.

54. The compound of any one of claims 51-53, or a pharmaceutically acceptable salt, solvate, or stereoisomer thereof, wherein R^{X} is hydrogen.

55. The compound of any one of claims 51-54, or a pharmaceutically acceptable salt, solvate, or stereoisomer thereof, wherein R^{Y} is hydrogen.

56. The compound of any one of claims 51-55, or a pharmaceutically acceptable salt, solvate, or stereoisomer thereof, wherein each R¹ is independently deuterium, halogen, C₁-C₆alkyl, or C₁-C₆haloalkyl.

57. The compound of any one of claims 51-56, or a pharmaceutically acceptable salt, solvate, or stereoisomer thereof, wherein n is 0 or 1.

58. The compound of any one of claims 51-57, or a pharmaceutically acceptable salt, solvate, or stereoisomer thereof, wherein R² is cycloalkyl or heterocycloalkyl; wherein the cycloalkyl and heterocycloalkyl is optionally substituted with one or more R^{2a}.

59. The compound of any one of claims 51-57, or a pharmaceutically acceptable salt, solvate, or stereoisomer thereof, wherein R² is aryl or heteroaryl; wherein the aryl and heteroaryl is optionally substituted with one or more R^{2a}.

60. The compound of any one of claims 51-59, or a pharmaceutically acceptable salt, solvate, or stereoisomer thereof, wherein each R^{2a} is independently deuterium, halogen, —CN, —OH, —OR^{a}, —S(═O)₂R^{a}, —NR^{c}R^{d}, C₁-C₆alkyl, or C₁-C₆haloalkyl.

61. The compound of any one of claims 51-60, or a pharmaceutically acceptable salt, solvate, or stereoisomer thereof, wherein R⁶ is hydrogen.

62. The compound of any one of claims 51-61, or a pharmaceutically acceptable salt, solvate, or stereoisomer thereof, wherein each R⁷ is independently hydrogen, deuterium, halogen, C₁-C₆alkyl, C₁-C₆haloalkyl, or C₁-C₆deuteroalkyl.

63. The compound of any one of claims 51-62, or a pharmaceutically acceptable salt, solvate, or stereoisomer thereof, wherein each R⁷ is independently hydrogen, halogen, or C₁-C₆alkyl.

64. The compound of any one of claims 51-63, or a pharmaceutically acceptable salt, solvate, or stereoisomer thereof, wherein R^{Z1} is hydrogen.

65. The compound of any one of claims 51-64, or a pharmaceutically acceptable salt, solvate, or stereoisomer thereof, wherein R^{Z2} is hydrogen.

66. A compound of Formula (V), or a pharmaceutically acceptable salt, solvate, or stereoisomer thereof: 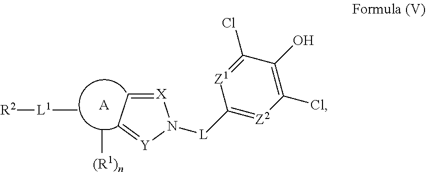
wherein:
Ring A is phenyl or 6-membered heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
n is 0-3;
L¹ is absent, —O—, —S—, —NR⁶—, —C(═O)—, —C(═O)O—, —S(═O)—, —S(═O)₂—, —NR⁶—C(R⁷)₂—, —C(R⁷)₂—NR⁶—, —NR⁶—C(═O)—, or —C(═O)—NR⁶—;
R² is cycloalkyl, heterocycloalkyl, aryl, or heteroaryl; wherein the cycloalkyl, heterocycloalkyl, aryl, and heteroaryl is optionally substituted with one or more R^{2a};
each R^{2a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{2a} on the same atom are taken together to form an oxo;
X is —N— or —CR^{X}—;
R^{X} is hydrogen, deuterium, halogen, —CN, —OH, —OR^{a}, —NR^{c}R^{d}, or C₁-C₆alkyl optionally substituted with one or more R;
Y is —N— or —CR^{Y}—;
R^{Y} is hydrogen, deuterium, halogen, —CN, —OH, —OR^{a}, —NR^{c}R^{d}, or C₁-C₆alkyl optionally substituted with one or more R;
L is absent, —C(R⁷)₂—, —C(═O)—, —S(═O)—, —S(═O)₂—, —C(R⁷)₂—NR⁶—, or —C(═O)—NR⁶—;
R⁶ is hydrogen or C₁-C₆alkyl optionally substituted with one or more R;
each R⁷ is independently hydrogen, deuterium, halogen, —CN, —OH, —OR^{a}, —NR^{c}R^{d}, or C₁-C₆alkyl optionally substituted with one or more R;
or two R⁷ are taken together to form an oxo;
or two R⁷ are taken together to form a cycloalkyl or heterocycloalkyl; each optionally substituted with one or more R;
Z¹ is N or CR^{Z1};
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z2};
R^{Z2} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

67. The compound of claim 66, or a pharmaceutically acceptable salt, solvate, or stereoisomer thereof, wherein the compound is of Formula (Va): 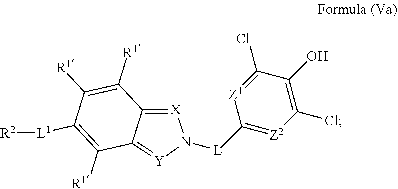
wherein each R^{1′} is independently hydrogen or R¹.

68. The compound of claim 66, or a pharmaceutically acceptable salt, solvate, or stereoisomer thereof, wherein the compound is of Formula (Vb): 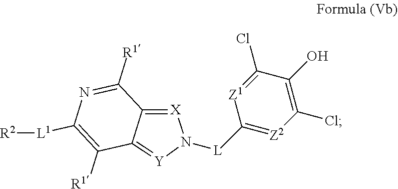
wherein each R^{1′} is independently hydrogen or R¹.

69. The compound of any one of claims 66-68, or a pharmaceutically acceptable salt, solvate, or stereoisomer thereof, wherein R^{X} is hydrogen.

70. The compound of any one of claims 66-69, or a pharmaceutically acceptable salt, solvate, or stereoisomer thereof, wherein R^{Y} is hydrogen.

71. The compound of any one of claims 66-70, or a pharmaceutically acceptable salt, solvate, or stereoisomer thereof, wherein each R¹ is independently deuterium, halogen, C₁-C₆alkyl, or C₁-C₆haloalkyl.

72. The compound of any one of claims 66-71, or a pharmaceutically acceptable salt, solvate, or stereoisomer thereof, wherein n is 0 or 1.

73. The compound of any one of claims 66-72, or a pharmaceutically acceptable salt, solvate, or stereoisomer thereof, wherein R² is cycloalkyl or heterocycloalkyl; wherein the cycloalkyl and heterocycloalkyl is optionally substituted with one or more R^{2a}.

74. The compound of any one of claims 66-72, or a pharmaceutically acceptable salt, solvate, or stereoisomer thereof, wherein R² is aryl or heteroaryl; wherein the aryl and heteroaryl is optionally substituted with one or more R^{2a}.

75. The compound of any one of claims 66-74, or a pharmaceutically acceptable salt, solvate, or stereoisomer thereof, wherein each R^{2a} is independently deuterium, halogen, —CN, —OH, —OR^{a}, —S(═O)₂R^{a}, —NR^{c}R^{d}, C₁-C₆alkyl, or C₁-C₆haloalkyl.

76. The compound of any one of claims 66-75, or a pharmaceutically acceptable salt, solvate, or stereoisomer thereof, wherein R⁶ is hydrogen.

77. The compound of any one of claims 66-76, or a pharmaceutically acceptable salt, solvate, or stereoisomer thereof, wherein each R⁷ is independently hydrogen, halogen, or C₁-C₆alkyl.

78. The compound of any one of claims 66-77, or a pharmaceutically acceptable salt, solvate, or stereoisomer thereof, wherein R^{Z1} is hydrogen.

79. The compound of any one of claims 66-78, or a pharmaceutically acceptable salt, solvate, or stereoisomer thereof, wherein R^{Z2} is hydrogen.

80. A compound of Formula (VIa) or (VIb), or a pharmaceutically acceptable salt, solvate, or stereoisomer thereof: 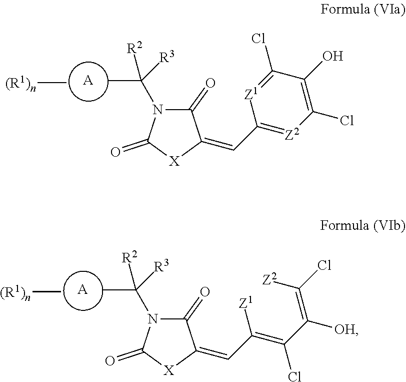
wherein:
Ring A is cycloalkyl, heterocycloalkyl, aryl, or heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or 2 R¹ on the same atoms are taken together to form an oxo;
n is 0-4;
R² is hydrogen, deuterium, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl;
R³ is hydrogen, deuterium, or C₁-C₆alkyl;
X is —O— or —S—;
Z¹ is N or CR^{Z1};
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z2};
R^{Z2} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

81. The compound of claim 80, or a pharmaceutically acceptable salt, solvate, or stereoisomer thereof, wherein the compound is of Formula (VIa):

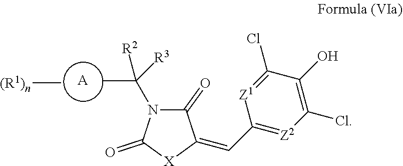

82. The compound of claim 80, or a pharmaceutically acceptable salt, solvate, or stereoisomer thereof, wherein the compound is of Formula (VIb):

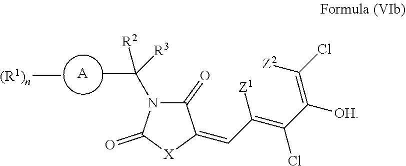

83. The compound of any one of claims 80-82, or a pharmaceutically acceptable salt, solvate, or stereoisomer thereof, wherein each R¹ is independently deuterium, halogen, —CN, —OH, —OR^{a}, C₁-C₆alkyl, or C₁-C₆haloalkyl.

84. The compound of any one of claims 80-83, or a pharmaceutically acceptable salt, solvate, or stereoisomer thereof, wherein n is 0 or 1.

85. The compound of any one of claims 80-84, or a pharmaceutically acceptable salt, solvate, or stereoisomer thereof, wherein R² is hydrogen.

86. The compound of any one of claims 80-85, or a pharmaceutically acceptable salt, solvate, or stereoisomer thereof, wherein R³ is hydrogen.

87. The compound of any one of claims 80-86, or a pharmaceutically acceptable salt, solvate, or stereoisomer thereof, wherein R^{Z1} is hydrogen.

88. The compound of any one of claims 80-87, or a pharmaceutically acceptable salt, solvate, or stereoisomer thereof, wherein R^{Z2} is hydrogen.

89. A compound of Formula (VII), or a pharmaceutically acceptable salt, solvate, or stereoisomer thereof: 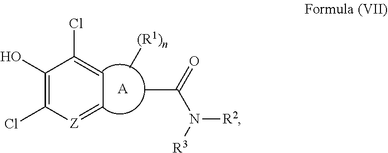
wherein:
Ring A is cycloalkyl, heterocycloalkyl, aryl, or heteroaryl;
each R¹ is independently deuterium, halogen, —CN, —NO₂, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R¹ on the same atom are taken together to form an oxo;
n is 0-4;
R² is hydrogen or C₁-C₆alkyl;
R³ is hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein the alkyl, alkylene, alkenyl, alkynyl, cycloalkyl, heterocycloalkyl, aryl, and heteroaryl is optionally substituted with one or more R^{3a};
each R^{3a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{3a} on the same atom are taken together to form an oxo;
Z is N or CR^{Z};
R^{Z} is hydrogen, deuterium, halogen, —CN, —OH, —OR^{a}, —NR^{c}R^{d}, or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo.

90. The compound of claim 89, or a pharmaceutically acceptable salt, solvate, or stereoisomer thereof, wherein each R¹ is independently halogen, C₁-C₆alkyl, or C₁-C₆haloalkyl.

91. The compound of claim 89 or 90, or a pharmaceutically acceptable salt, solvate, or stereoisomer thereof, wherein n is 0 or 1.

92. The compound of any one of claims 89-91, or a pharmaceutically acceptable salt, solvate, or stereoisomer thereof, wherein R² is hydrogen.

93. The compound of any one of claims 89-92, or a pharmaceutically acceptable salt, solvate, or stereoisomer thereof, wherein each R^{3a} is independently deuterium, halogen, —CN, —OH, —OR^{a}, —NR^{c}R^{d}, C₁-C₆alkyl, or C₁-C₆haloalkyl.

94. The compound of any one of claims 89-93, or a pharmaceutically acceptable salt, solvate, or stereoisomer thereof, wherein R^{Z} is hydrogen.

95. A compound of Formula (VIIIa) or (VIIb), or a pharmaceutically acceptable salt, solvate, or stereoisomer thereof: 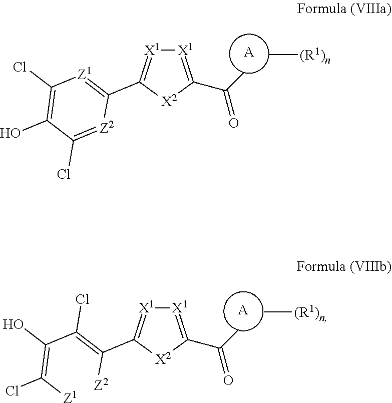
wherein:
Ring A is cycloalkyl, heterocycloalkyl, aryl, or heteroaryl;
each R¹ is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R¹ on the same atom are taken together to form an oxo;
n is 0-4;
Z¹ is N or CR^{Z1};
R^{Z1} is hydrogen, deuterium, halogen, —CN, —OH, —OR^{a}, —NR^{c}R^{d}, or C₁-C₆alkyl optionally substituted with one or more R;
Z² is N or CR^{Z1};
R^{Z2} is hydrogen, deuterium, halogen, —CN, —OH, —OR^{a}, —NR^{c}R^{d}, or C₁-C₆alkyl optionally substituted with one or more R;
each X¹ is independently —N— or —CR^{X1}—;
each R^{X1} is hydrogen, deuterium, halogen, —CN, —OH, —OR^{a}, —NR^{c}R^{d}, or C₁-C₆alkyl optionally substituted with one or more R;
or two R^{X1} are taken together to form a cycloalkyl, heterocycloalkyl, aryl, or heteroaryl; each optionally substituted with one or more R;
X² is —O—, —S—, or —NR^{X2}—;
R^{X2} is hydrogen or C₁-C₆alkyl optionally substituted with one or more R;
each R^{a} is independently C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkylene(cycloalkyl), C₁-C₆alkylene(heterocycloalkyl), C₁-C₆alkylene(aryl), or C₁-C₆alkylene(heteroaryl); wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R;
each R is independently halogen, —CN, —OH, —OC₁-C₃alkyl, —OC₁-C₃haloalkyl, —SC₁-C₃alkyl, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —NH₂, —NHC₁-C₃alkyl, —N(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, C₁-C₃haloalkyl, C₁-C₃deuteroalkyl, C₁-C₃hydroxyalkyl, C₁-C₃aminoalkyl, C₁-C₃heteroalkyl, or C₃-C₆cycloalkyl;
or two R on the same atom are taken together to form an oxo;
provided that the compound is not:

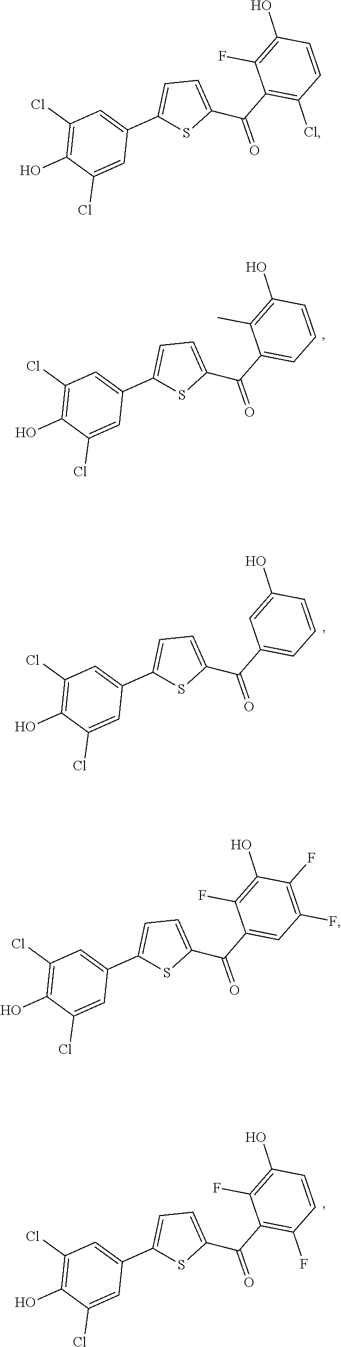 [SMILES: O=C(c1ccc(-c2cc(Cl)c(O)c(Cl)c2)s1)c1c(Cl)ccc(O)c1F ; Cc1c(O)cccc1C(=O)c1ccc(-c2cc(Cl)c(O)c(Cl)c2)s1 ; O=C(c1cccc(O)c1)c1ccc(-c2cc(Cl)c(O)c(Cl)c2)s1 ; O=C(c1ccc(-c2cc(Cl)c(O)c(Cl)c2)s1)c1cc(F)c(F)c(O)c1F ; O=C(c1ccc(-c2cc(Cl)c(O)c(Cl)c2)s1)c1c(F)ccc(O)c1F]

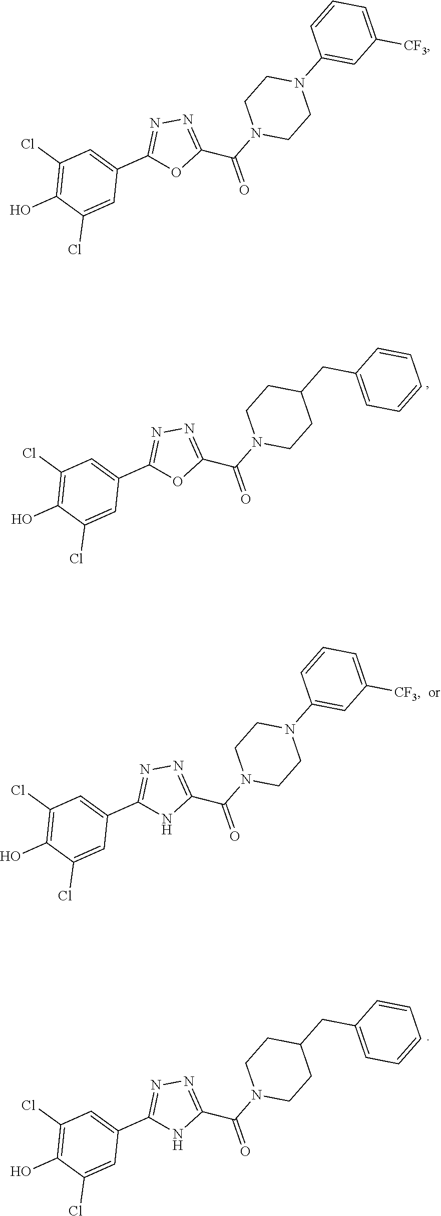

96. The compound of claim 95, or a pharmaceutically acceptable salt, solvate, or stereoisomer thereof, wherein the compound is of Formula (VIIIa):

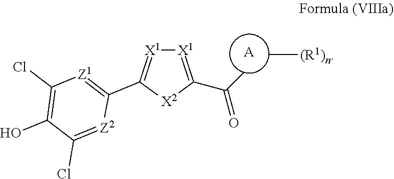

97. The compound of claim 95, or a pharmaceutically acceptable salt, solvate, or stereoisomer thereof, wherein the compound is of Formula (VIIIb):

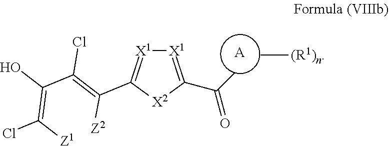

98. The compound of any one of claims 95-97, or a pharmaceutically acceptable salt, solvate, or stereoisomer thereof, wherein each R¹ is independently deuterium, halogen, —CN, —OH, —OR^{a}, C₁-C₆alkyl, or C₁-C₆haloalkyl.

99. The compound of any one of claims 95-98, or a pharmaceutically acceptable salt, solvate, or stereoisomer thereof, wherein n is 0-3.

100. The compound of any one of claims 95-99, or a pharmaceutically acceptable salt, solvate, or stereoisomer thereof, wherein R^{Z1} is hydrogen.

101. The compound of any one of claims 95-100, or a pharmaceutically acceptable salt, solvate, or stereoisomer thereof, wherein R^{Z2} is hydrogen.

102. The compound of any one of claims 95-101, or a pharmaceutically acceptable salt, solvate, or stereoisomer thereof, wherein each R^{X1} is hydrogen, deuterium, halogen, or C₁-C₆alkyl optionally substituted with one or more R.

103. The compound of any one of claims 95-102, or a pharmaceutically acceptable salt, solvate, or stereoisomer thereof, wherein each R^{X1} is hydrogen or C₁-C₆alkyl.

104. The compound of any one of claims 95-103, or a pharmaceutically acceptable salt, solvate, or stereoisomer thereof, wherein each R^{X1} is hydrogen.

105. The compound of any one of claims 95-104, or a pharmaceutically acceptable salt, solvate, or stereoisomer thereof, wherein R^{X2} is hydrogen or C₁-C₆alkyl.

106. A compound selected from a compound found in the specification, or a pharmaceutically acceptable salt, solvate, or stereoisomer thereof.

107. A pharmaceutical composition comprising a compound of any one of claims 1-106, or a pharmaceutically acceptable salt, solvate, or stereoisomer thereof, and a pharmaceutically acceptable carrier.

108. A method of treating a disease in a subject in need thereof, the method comprising administering a pharmaceutically effective amount of a compound of any one of claims 1-106, or a pharmaceutically acceptable salt, solvate, or stereoisomer thereof, or a pharmaceutical composition of claim 107.

109. The method of claim 108, wherein the disease is a liver disease, a metabolic disease, or a cardiovascular disease.

110. The method of claim 108 or 109, wherein the disease is NAFLD.

111. The method of claim 108 or 109, wherein the disease is NASH.

112. The method of claim 108 or 109, wherein the disease is drug induced liver injury (DILI).

113. The method of claim 108 or 109, wherein the disease is associated with HSD17B13.

114. The method of claim 108 or 109, wherein the diseases is alcoholic liver disease.

115. The method of claim 108 or 109, wherein the disease is cirrhosis.

116. The method of claim 108 or 109, wherein the disease is decompensated portal hypertension.

117. The method of claim 108 or 109, wherein the disease is cholestatic liver disease.
